(19) United States
(12) Patent Application Publication
(10) Pub. No.: US 2006/0263784 A1
(43) Pub. Date: Nov. 23, 2006

(54) Method for the diagnosis/prognosis of neuroblastoma

(76) Inventors: Valerie Combaret, Lyon (FR); Alexander Krause, Lyon (FR); Alain Puisieux, Ruy Montceau (FR); Bruno Lacroix, SaintGenis Laval (FR)

Correspondence Address: OLIFF & BERRIDGE, PLC, P.O. BOX 19928, ALEXANDRIA, VA, 22320, US

(21) Appl. No.: 10/572,905

(22) PCT Filed: Oct. 1, 2004
(86) PCT No.: PCT/FR04/50475

§ 371 Date: Mar. 21, 2006

(30) Foreign Application Priority Data
Oct. 1, 2003 (FR) 0311483

(51) Int. Cl. C12Q 1/68; C12M 1/34

(52) U.S. Cl. 435/6; 435/287.2

(57) ABSTRACT

The present invention relates to a method for neuroblastoma prognosis in a patient suffering from neuroblastoma, characterized in that it comprises the following steps: a. biological material is extracted from a biological sample taken from the patient, b. the biological material is brought into contact with at least one specific reagent chosen from the reagents specific for the target genes exhibiting a nucleic acid sequence having any one of SEQ ID Nos. 1 to 37, it being understood that, when the target gene exhibits a nucleic acid sequence having one of SEQ ID Nos. 11, 17 or 37, the biological material is brought into contact with at least two specific reagents chosen from the reagents specific for the target genes exhibiting a nucleic acid sequence having any one of SEQ ID Nos. 1 to 37, c. the expression of at least one of said target genes is determined, it being understood that, when the target gene exhibits a nucleic acid sequence having one of SEQ ID Nos. 11, 17 or 37, the expression of at least two of said target genes is determined.

The present invention relates to a method for neuroblastoma prognosis.

Neuroblastoma is the second most common cause of solid tumour in children after brain tumours. Neuroblastoma is the most common of cancers in children younger than five years old and represents approximately 15% of cancers before this age.

Neuroblastomas are malignant tumours which have developed from neuroblasts derived from the neural crest and that migrate so as to form the sympathetic ganglia and the adrenal medulla during the embryonic and foetal period.

When a first clinical examination leads to a neuroblastoma being suspected (lump, haematoma, painful area, difficulty moving the limbs, etc.), a complete assessment is carried out in order to confirm the diagnosis.

In general, this assessment comprises:
tests on samples (blood, urine),
various radiological examinations, the aim of which is to correctly locate the tumour, its limits and its size (scintigraphy, echography and/or scan and/or MRI),
examinations of tumour fragments under a microscope in order to discover exactly what type of tumour is involved.

At the current time, no universal treatment exists when a neuroblastoma is diagnosed, and a specific treatment must be adapted according to the age of the patient. Local-regional treatments (surgery and radiotherapy) in order to remove or destroy the tumour directly, at the site where it is found and general treatments (chemotherapy), which act throughout the patient's body, both on the tumour but also where there may be metastases, are therefore mainly distinguished.

The patient's treatment can be adjusted according to the prognosis of the neuroblastoma and the therapeutic strategy can prove to be very different depending on the stage and the genetic characterization of the tumour cells. Thus, in localized forms in which the tumour cells bear no characteristic of a poor prognosis, the treatment is essentially surgical, whereas in localized forms in which the tumour cells give a poor prognosis, the treatment must be more aggressive, and based on chemotherapy and local radiotherapy.

At the current time various neuroblastoma classifications exist that make it possible to define prognostic groups as specifically as possible. These groups theoretically make it possible to define the therapeutic indications in a manner that is adapted to the risk of the disease. Mention may in particular be made of the classification of the International Neuroblastoma Staging System (Brodeur et al. (1993) J. Clin. Oncol. 11, 1466-77), which takes into account the anatomical data currently recognized as having a prognostic value. According to this classification, the following stages are distinguished:
stage 1: localized tumour with complete gross excision; ipsilateral and controlateral lymph nodes examined and negative microscopically,
stage 2A: unilateral tumour with incomplete excision, ipsilateral and controlateral lymph nodes examined and negative;
stage 2B: unilateral tumour with ipsilateral lymph node involvement but controlateral lymph nodes negative;
stage 3: inoperable unilateral tumour infiltrating across the midline, or unilateral tumour with controlateral lymph node involvement, or midline tumour with bilateral extension by infiltration or by adenopathy;
stage 4: primary tumour with distant dissemination: to lymph nodes, bone, bone marrow, liver;
stage 4S: local stage 1 or 2 tumour with dissemination limited to liver, skin or bone marrow. The 4S stages are children less than 1 year old.

Currently, the prognosis for a neuroblastoma can be established by studying various factors:
1) Amplification of the N-myc oncogene is considered to be a reference tool, and is used by most paediatric oncologists to define, at the time of diagnosis, the patients which must receive intensive chemotherapy followed by a bone marrow graft (Seeger et al, N Engl J Med. 1985; 313(18):1111-6; Rubie et al J Clin Oncol. 1997 March;15(3):1171-82.).
2) There is also thought to exist a correlation between the prognosis for neuroblastoma and the VMA (vanilmandelic acid)/HVA (homovanillic acid) catecholamine ratio, at the time of diagnosis. In advanced stages, a high urinary excretion of HVA and low, even normal, urinary excretion of VMA would indicate a poor prognosis (Laug et al Pediatrics. 1978; 62(1):77-83).
3) The increase in serum ferritin in neuroblastomas is also considered to be a factor for poor prognosis (Evans et al, Cancer. 1987; 59(11):1853-9).
4) The LDH (lactate dehydrogenase) level could also be an independent and predominant prognostic factor for localized stages I to III in children more than one year old, and, to a lesser extent, in children less than one year old with a stage IV (Berthold et al, Am J Pediatr Hematol Oncol. 1994; 16(2):107-15).

However, the correlation between amplification of the N-myc oncogene and the prognosis for neuroblastoma is not absolute (Maris & Matthay, J Clin Oncol, 1999, 17(7): 2264-2279). Furthermore, since LDH and ferritin are two factors that correlate with one another, the reliability of these factors for neuroblastoma prognosis remains disputed (Berthold et al, 1992, Am J Pediatr Hamtol Oncol, 14(3): 207-215). Finally, the use of the VMA/HVA ratio gives a sensitivity and a specificity that are insufficient for neuroblastoma prognosis.

The present invention proposes to solve all the drawbacks of the state of the art by providing a novel tool for neuroblastoma prognosis.

Surprisingly, the inventors have demonstrated that the prognosis of a neuroblastoma can be determined by analyzing the expression of target genes selected from 37 genes as shown in Table 1 hereinafter, which are expressed differentially depending on whether the patient has a good or a poor prognosis.

TABLE 1
List of the 37 target genes according to the invention

| SEQ | | |
|---|---|---|
| ID | | Genbank |
| No. | Sequence description | No. |
| 1 | Flap structure-specific endonuclease 1 (FEN1) | NM_004111 |
| 2 | Ubiquitin-conjugating enzyme E2C (UBE2C) | NM_007019 |
| 3 | Insulin-like growth factor binding protein 7 | NM_001553 |
| | (IGFBP7) | |
| 4 | Collagen, type I, alpha 2 (COL1A2) | NM_000089 |
| 5 | Nucleolin (NCL) | NM_005381 |
| 6 | Interleukin enhancer binding factor 3, 90 kDa | NM_004516 |
| | (ILF3), transcript variant 2 | |
| 7 | cDNA FLJ30781 fis, clone FEBRA2000874, | AK055343/ |
| | corresponding to the gene encoding adenylate | NM_021116 |
| | cyclase 1 (brain) | |
| 8 | TIF1 beta zinc finger protein | X97548 |
| 9 | Likely orthologue of mouse tumour | NM_020755 |
| | differentially expressed 1, like (TDE1L) | |
| 10 | DKFZp434D1112_s1 434 (synonym: htes3) cDNA | AL039831 |
| | clone DKFZp434D1112 3′ | |
| 11 | v-myc myelocytomatosis viral related oncogene, | NM_005378 |
| | neuroblastoma derived (avian)(MYCN) | |
| 12 | Small nuclear ribonucleoprotein D2 polypeptide | NM_004597 |
| | 16.5 kDa (SNRPD2), transcript variant 1 | |
| 13 | MCM2 minichromosome maintenance deficient 2, | NM_004526 |
| | mitotin (S. cerevisiae) | |
| 14 | RuvB-like 2 (E. coli) (RUVBL2) | NM_006666 |
| 15 | Immediate early protein ETR101 | NM_004907 |
| 16 | RNA binding protein S1, serine-rich domain | NM_006711 |
| | (RNPS1), transcript variant 1 | |
| 17 | Ornithine decarboxylase 1 (ODC1) | NM_002539 |
| 18 | Activity-regulated cytoskeleton-associated | NM_015193 |
| | protein (ARC) | |
| 19 | Secretogranin II (chromogranin C) (SCG2) | NM_003469 |
| 20 | Structure specific recognition protein 1 | NM_003146 |
| | (SSRP1) | |
| 21 | Collagen, type VI, alpha 3 (COL6A3), | NM_004369 |
| | transcript variant 1 | |
| 22 | Small nuclear ribonucleoprotein polypeptides | NM_003091 |
| | B and B1 (SNRPB) | |
| 23 | Acidic (leucine-rich) nuclear phosphoprotein | NM_006401 |
| | 32 family, member B (ANP32B) | |
| 24 | Non-POU domain containing, octamer-binding | NM_007363 |
| | (NONO) | |
| 25 | Peripheral myelin protein 22 (PMP22), | NM_000304 |
| | transcript variant 1 | |
| 26 | Small nuclear ribonucleoprotein polypeptide E | NM_003094 |
| | (SNRPE) | |
| 27 | KIAA0436 mRNA, partial cds | AB007896 |
| 28 | Fibrillarin (FBL) | NM_001436 |
| 29 | Tripartite motif-containing 2 (TRIM2) | NM_015271 |
| 30 | MCM6 minichromosome maintenance deficient 6 | NM_005915 |
| | (MIS5 homologue, S. pombe) (S. cerevisiae) | |
| | (MCM6) | |
| 31 | Polypyrimidine tract binding protein 1 | NM_002819 |
| | (PTBP1), transcript variant 1 | |
| 32 | Small nuclear ribonucleoprotein polypeptide | NM_004596 |
| | A (SNRPA) | |
| 33 | Creatine kinase, brain (CKB) | NM_001823 |
| 34 | Erythrocyte membrane protein band 4.1-like | NM_012307 |
| | 3 (EPB41L3) | |
| 35 | Hypothetical protein MGC3077 | NM_024051 |
| 36 | Tissue alpha-L-fucosidase 1 (FUCA 1) | NM_000147 |
| 37 | Secreted protein acidic and rich in cysteine | NM_003118 |
| | (SPARC) | |

Among these genes, genes whose function is known but which have never been related to neuroblastoma (SEQ ID No. 1 to 8; 12 to 16; 18 to 26; 28; 30 to 34; 36), and also genes whose function is unknown (SEQ ID No. 9; 10; 27; 29; 35), can be distinguished. It is clearly understood that if various isoforms of these genes exist, all the isoforms are relevant for the present invention, and not only those given in the above table.

To this effect, the present invention relates to a method for neuroblastoma prognosis in a patient suffering from neuroblastoma, characterized in that it comprises the following steps:
a. biological material is extracted from a biological sample taken from the patient,
b. the biological material is brought into contact with at least one specific reagent chosen from the reagents specific for the target genes exhibiting a nucleic acid sequence having any one of SEQ ID Nos. 1 to 37, it being understood that, when the target gene exhibits a nucleic acid sequence having one of SEQ ID Nos. 11, 17 or 37, the biological material is brought into contact with at least two specific reagents chosen from the reagents specific for the target genes exhibiting a nucleic acid sequence having any one of SEQ ID Nos. 1 to 37,
c. the expression of at least one of said target genes is determined, it being understood that, when the target gene exhibits a nucleic acid sequence having one of SEQ ID Nos. 1, 17 or 37, the expression of at least two of said target genes is determined.

For the purposes of the present invention, the term biological sample is intended to mean any sample taken from a patient, and which may contain a biological material as defined hereinafter. This biological sample can in particular be a sample of blood, serum, saliva, tissue, tumour, bone marrow or circulating cells from the patient. This biological sample is provided by any type of means for taking a sample known to those skilled in the art. According to a preferred embodiment of the invention, the biological sample taken from the patient is a tissue sample, preferably a tumour or bone marrow sample.

For the purposes of the present invention, the term biological material is intended to mean any material that makes it possible to detect the expression of a target gene. The biological material can in particular comprise proteins, or nucleic acids such as, in particular, deoxyribonucleic acids (DNA) or ribonucleic acids (RNA). The nucleic acid can in particular be an RNA (ribonucleic acid). According to a preferred embodiment of the invention, the biological material comprises nucleic acids, preferably RNAs, and even more preferably total RNAs. The total RNAs comprise the transfer RNAs, the messenger RNAs (mRNAs), such as the mRNAs transcribed from the target gene, but also transcribed from any other gene and the ribosomal RNAs. This biological material comprises material specific for a target gene, such as in particular the mRNAs transcribed from the target gene or the proteins derived from these mRNAs, but can also comprise material not specific for a target gene, such as in particular the mRNAs transcribed from a gene other than the target gene, the tRNAs, or the rRNAs derived from genes other than the target gene.

During step a) of the method according to the invention, the biological material is extracted from the biological sample by any of the nucleic acid extraction and purification protocols well known to those skilled in the art.

By way of indication, the nucleic acid extraction can be carried out by means of:
a step consisting of lysis of the cells present in the biological sample, in order to release the nucleic acids contained in the cells of the patient. By way of example, it is possible to use the lysis methods as described in patent applications:
   WO 00/05338 regarding mixed magnetic and mechanical lysis,
   WO 99/53304 regarding electrical lysis,
   WO 99/15321 regarding mechanical lysis.
Those skilled in the art may use other well known methods of lysis, such as thermal or osmotic shocks or chemical lysis with chaotropic agents such as guanidium salts (U.S. Pat. No. 5,234,809);
a purification step, allowing separation between the nucleic acids and the other cellular constituents released in the lysis step. This step generally makes it possible to concentrate the nucleic acids, and can be adapted to the purification of DNA or of RNA. By way of example, it is possible to use magnetic particles optionally coated with oligonucleotides, by adsorption or covalence (in this respect, see patents U.S. Pat. No. 4,672,040 and U.S. Pat. No. 5,750,338), and thus to purify the nucleic acids which are bound to these magnetic particles, by means of a washing step. This nucleic acid purification step is particularly advantageous if it is desired to subsequently amplify said nucleic acids. A particularly advantageous embodiment of these magnetic particles is described in patent applications: WO-A-97/45202 and WO-A-99/35500. Another advantageous example of a method of purifying nucleic acids is the use of silica either in the form of a column, or in the form of inert particles (Boom R. et al., J. Clin. Microbiol.; 1990, No. 28(3), p. 495-503) or magnetic particles (Merck: MagPrep® Silica, Promega: MagneSil™ Paramagnetic particles). Other, very widespread, methods are based on ion exchange resins in a column or in a paramagnetic particulate format (Whatman: DEAE-Magarose) (Levison P R et al., J. Chromatography, 1998, p. 337-344). Another very relevant, but not exclusive, method for the invention is that of adsorption onto a metal oxide support (from the company Xtrana: Xtra-Bind™ matrix).
When it is desired to specifically extract the DNA from a biological sample, it is in particular possible to carry out an extraction with phenol, chloroform and alcohol in order to remove the proteins, and to precipitate the DNA with 100% ethanol. The DNA can then be pelleted by centrifugation, washed, and redissolved.
When it is desired to specifically extract the RNAs from a biological sample, it is in particular possible to carry out an extraction with phenol, chloroform and alcohol in order to remove the proteins, and to precipitate the RNAs with 100% ethanol. The RNAs can then be pelleted by centrifugation, washed, and redissolved.

For the purpose of the present invention, the term specific reagent is intended to mean a reagent which, when it is brought into contact with the biological material as defined above, binds with the material specific to said target gene. By way of indication, when the specific reagent and the biological material are of nucleic acid origin, bringing the specific reagent into contact with the biological material allows the specific reagent to hybridize with the material specific for the target gene. The term hybridization is intended to mean the process during which, under appropriate conditions, two nucleotide fragments bind to one another with stable and specific hydrogen bonds so as to form a double-stranded complex. These hydrogen bonds form between the complementary bases adenine (A) and thymine (T) (or uracil (U)) (described as an A-T bond) or between the complementary bases guanine (G) and cytosine (C) (described as a G-C bond). The hybridization of two nucleotide fragments can be complete (then described as complementary nucleotide fragments or sequences), i.e. the double-stranded complex obtained during this hybridization comprises only A-T bonds and C-G bonds. This hybridization can be partial (then described as sufficiently complementary nucleotide fragments or sequences), i.e. the double-stranded complex obtained comprises A-T bonds and C-G bonds that make it possible to form the double-stranded complex, but also bases that are not bound to a complementary base. The hybridization between two nucleotide fragments depends on the operating conditions that are used, and in particular on the stringency. The stringency is defined in particular as a function of the base composition of the two nucleotide fragments, and also by the degree of mismatch between two nucleotide fragments. The stringency may also depend on the parameters of the reaction, such as the concentration and the type of ionic species present in the hybridization solution, the nature and the concentration of denaturing agents and/or the hybridization temperature. All these data are well known and the appropriate conditions can be determined by those skilled in the art. In general, depending on the length of the nucleotide fragments that it is desired to hybridize, the hybridization temperature is between approximately 20 and 70° C., in particular between 35 and 65° C., in a saline solution at a concentration of approximately 0.5 to 1 M. A sequence, or a nucleotide fragment, or an oligonucleotide, or polynucleotide, is a chain of nucleotide units assembled together via phosphoric ester bonds, characterized by the informational sequence of the natural nucleic acids, capable of hybridizing to a nucleotide fragment, it being possible for the chain to contain monomers having different structures and to be obtained from a natural nucleic acid molecule and/or by genetic recombination and/or by chemical synthesis. A unit is derived from a monomer which can be a natural nucleotide of a nucleic acid, the constitutive elements of which are a sugar, a phosphate group and a nitrogenous base; in DNA, the sugar is deoxy-2-ribose, in RNA, the sugar is ribose; depending on whether it is a question of DNA or RNA, the nitrogenous base is chosen from adenine, guanine, uracil, cytosine and thymine; or alternatively the monomer is a nucleotide that has been modified in at least one of the three constitutive elements; by way of example, the modification can occur either at the level of the bases, with modified bases such as inosine, methyl-5-deoxycytidine, deoxyuridine, diinethylamino-5-deoxyuridine, diamino-2,6-purine, bromo-5-deoxyuridine or any other modified base capable of hybridization, or at the level of the sugar, for example the replacement of at least one deoxyribose with a polyamide (P. E. Nielsen et al, Science, 254, 1497-1500 (1991), or else at the level of the phosphate group, for example its replacement with esters, in particular chosen from diphosphates, alkylphosphonates, arylphosphonates and phosphorothioates.

According to one specific embodiment of the invention, the specific reagent comprises at least one amplification primer. For the purpose of the present invention, the term amplification primer is intended to mean a nucleotide fragment comprising from 5 to 100 nucleic units, preferably from 15 to 30 nucleic units, allowing the initiation of an enzymatic polymerization, such as in particular an enzymatic amplification reaction. According to one specific embodiment of the invention, the amplification primer comprises a sequence chosen from SEQ ID Nos. 38 to 41 and SEQ ID Nos. 44 and 45. The term enzymatic amplification reaction is intended to mean a process generating multiple copies of a nucleotide fragment by the action of at least one enzyme. Such amplification reactions are well known to those skilled in the art and mention may in particular be made of the following techniques:
PCR (Polymerase Chain Reaction), as described in patents U.S. Pat. No. 4,683,195, U.S. Pat. No. 4,683,202 and U.S. Pat. No. 4,800,159,
LCR (Ligase Chain Reaction), disclosed, for example, in patent application EP 0 201 184,
RCR Repair Chain Reaction), described in patent application WO 90/01069,
3SR (Self Sustained Sequence Replication) with patent application WO 90/06995,
NASBA (Nucleic Acid Sequence-Based Amplification) with patent application WO 91/02818, and
TMA (Transcription Mediated Amplification) with patent U.S. Pat. No. 5,399,491.

When the enzymatic amplification is a PCR, the specific reagent comprises at least 2 amplification primers specific for a target gene, and which allow the amplification of the material specific for the target gene. The material specific for the target gene then preferably comprises a complementary DNA obtained by reverse transcription of messenger RNA derived from the target gene (then described as cDNA specific for the target gene) or a complementary RNA obtained by transcription of the cDNA specific for a target gene (then described as cRNA specific for the target gene). When the enzymatic amplification is a PCR carried out after a reverse transcription reaction, it is referred to as RT-PCR.

According to another preferred embodiment of the invention, the specific reagent of step b) preferably comprises a hybridization probe.

The term hybridization probe is intended to mean a nucleotide fragment comprising from 5 to 100 nucleic units, in particular from 10 to 35 nucleic units, having a hybridization specificity under given conditions so as to form a hybridization complex with the material specific for a target gene. In the present invention, the material specific for the target gene can be a nucleotide sequence included in a messenger RNA derived from the target gene (reference is then made to an mRNA specific for the target gene), a nucleotide sequence included in a complementary DNA obtained by reverse transcription of said messenger RNA (reference is then made to a cDNA specific for the target gene), or else a nucleotide sequence included in a complementary RNA obtained by transcription of said cDNA as described above (reference will then be made to a cRNA specific for the target gene). The hybridization probe can comprise a label for its detection. The term detection is intended to mean either a direct detection by a physical method, or an indirect detection by a method of detection using a label. Many methods of detection exist for the detection of nucleic acids [see, for example, Kricka et al., Clinical Chemistry, 1999, No. 45(4), p. 453-458 or Keller G. H. et al., DNA Probes, 2nd Ed., Stockton Press, 1993, sections 5 and 6, p. 173-249]. The term label is intended to mean a tracer capable of engendering a signal that can be detected. A nonlimiting list of these tracers includes the enzymes which produce a signal detectable, for example, by colorimetry, fluorescence or luminescence, such as horseradish peroxydase, alkaline phosphatase, beta galactosidase, or glucose-6-phosphate dehydrogenase; chromophores, such as fluorescent, luminescent or dye compounds; electron-dense groups that can be detected by electron microscopy or by means of their electrical properties such as conductivity, by amperometry or voltammetry methods, or by impedance measurements; groups that can be detected by optical methods such as diffraction, surface plasmon resonance, or contact angle variation, or by physical methods such as atomic force spectroscopy, tunnel effect, etc.; and radioactive molecules such as ³²P, ³⁵S or ¹²⁵I.

For the purpose of the present invention, the hybridization probe may be a “detection” probe. In this case, the “detection” probe is labelled with a label as defined above. The hybridization probe can also be a “capture” probe. In this case, the “capture” probe is immobilized or can be immobilized on a solid support by any appropriate means, i.e. directly or indirectly, for example by covalence or adsorption. As a solid support, use may be made of synthetic materials or natural materials, that are optionally chemically modified, in particular polysaccharides, such as cellulose-based materials, for example paper, cellulose derivatives such as cellulose acetate and nitrocellulose or dextran, polymers, copolymers, in particular based on styrene-type monomers, natural fibres such as cotton, and synthetic fibres such as nylon; mineral materials such as silica, quartz, glasses or ceramics; latices; magnetic particles; metal derivatives, gels, etc. The solid support can be in the form of a microtitration plate, of a membrane as described in application WO-A-94/12670, or of a particle. It is also possible to immobilize several different capture probes on the support, each one being specific for a target gene. In particular, it is possible to use, as support, a biochip on which a large number of probes can be immobilized. The term biochip is intended to mean a solid support that is small in size and to which a multitude of capture probes are attached at predetermined positions. The biochip or DNA chip concept dates from the beginning of the 1990s. It is based on a pluridisciplinary technology integrating microelectronics, nucleic acid chemistry, image analysis and computer technology. The operating principle is based on a foundation of molecular biology: the hybridization phenomenon, i.e. the pairing by complementarity of the bases of two DNA and/or RNA sequences. The biochip method is based on the use of capture probes attached to a solid support, on which is reacted a sample of target nucleotide fragments labelled directly or indirectly with fluorochromes. The capture probes are positioned specifically on the support or chip and each hybridization gives a specific piece of information, in relation to the target nucleotide fragment. The pieces of information obtained are, cumulative, and make it possible, for example, to quantify the level of expression of a target gene or of several target genes. To analyse the expression of a target gene, it is therefore possible to prepare a biochip carrying a very large number of probes which correspond to all or part of the target gene, which is transcribed into mRNA. The cDNAs or the cRNAs specific for a target gene that it is desired to analyse, for example, are then hybridized on specific capture probes. After hybridization, the support or chip is washed, and the labelled cDNA or cRNA/capture probe complexes are revealed with a high-affinity ligand bound, for example, to a fluorochrome-type label. The fluorescence is read, for example, with a scanner and the analysis of the fluorescence is processed by computer technology. By way of indication, mention may be made of the DNA chips developed by the company Affymetrix (“Accessing Genetic Information with High-Density DNA arrays”, M. Chee et al., Science, 1996, 274, 610-614. “Light-generated oligonucleotide arrays for rapid DNA sequence analysis”, A. Caviani Pease et al., Proc. Natl. Acad. Sci. USA, 1994, 91, 5022-5026), for molecular diagnoses. In this technology, the capture probes are generally small in size, around 25 nucleotides. Other examples of biochips are given in the publications by G. Ramsay, Nature Biotechnology, 1998, No. 16, p. 40-44; F. Ginot, Human Mutation, 1997, No. 10, p. 1-10; J. Cheng et al, Molecular diagnosis, 1996, No. 1(3), p. 183-200; T. Livache et al, Nucleic Acids Research, 1994, No. 22(15), p. 2915-2921; J. Cheng et al, Nature Biotechnology, 1998, No. 16, p. 541-546 or in patents U.S. Pat. No. 4,981,783, U.S. Pat. No. 5,700,637, U.S. Pat. No. 5,445,934, U.S. Pat. No. 5,744,305 and U.S. Pat. No. 5,807,522. The main characteristic of the solid support must be that of conserving the characteristics of hybridization of the capture probes on the target nucleotide fragments while at the same time generating a minimum background noise for the method of detection.

For the immobilization of the probes on the support, three major types of fabrication are distinguished.

First of all, there is a first technique which consists in depositing presynthesized probes. The attachment of the probes occurs by direct transfer, by means of micropipettes or of microtips, or by means of an inkjet-type device. This technique makes it possible to attach probes having a size ranging from a few bases (5 to 10) up to relatively large sizes of 60 bases (printing) to a few hundred bases (microdeposition):

Printing is an adaptation of the method used by inkjet printers. It is based on the propulsion of very small spheres of fluid (volume<1 nl) at a rate that can reach 4000 drops/second. The printing does not involve any contact between the system releasing the fluid and the surface on which it is deposited.

Microdeposition consists in attaching long probes of from a few tens of bases to several hundred bases to the surface of a glass slide. These probes are generally extracted from databases and are in the form of amplified and purified products. This technique makes it possible to produce chips called microarrays that carry approximately ten thousand spots, called recognition zones, of DNA on a surface area of slightly less than 4 cm². The use of nylon membranes, called “macroarrays”, which carry amplified products, generally PCR-amplified products, with a diameter of 0.5 to 1 mm, and the maximum density of which is 25 spots/cm², should not, however, be forgotten. This very flexible technique is used by many laboratories. In the present invention, this latter technique is considered to be part of the biochips. It is, however, possible to deposit at the bottom of a microtitration plate a certain volume of sample in each well, as is the case in patent applications WO-A-00/71750 and FR 00/14896, or to deposit at the bottom of the same Petri dish a certain number of drops that are separated from one another, according to another patent application FR 00/14691.

The second technique for attaching the probes to the support or chip is called in situ synthesis. This technique results in the development of short probes directly at the surface of the chip. It is based on the synthesis of oligonucleotides in situ (see, in particular, patent applications WO 89/10977 and WO 90/03382), and is based on the oligonucleotide synthesizer method. It consists in moving a reaction chamber, in which the oligonucleotide elongation reaction takes place, along the glass surface.

Finally, the third technique is called photolithography, which is a process used for the biochips developed by Affymetrix. It is also an in situ synthesis. Photolithography is derived from microprocessor techniques. The surface of the chip is modified by the attachment of photolabile chemical groups that can be light-activated. Once illuminated, these groups are capable of reacting with the 3′ end of an oligonucleotide. By protecting this surface with masks of defined shapes, it is possible to selectively illuminate and therefore activate areas of the chip where it is desired to attach one or other of the four nucleotides. The successive use of different masks makes it possible to alternate cycles of protection/reaction and therefore to produce the oligonucleotide probes on spots of approximately a few tens of a micrometre squared. (μm²). This resolution makes it possible to create up to several hundred thousand spots on a surface area of a few centimetres squared (cm²). Photolithography has advantages: in bulk in parallel, it makes it possible to create a chip of N-mers in only 4×N cycles. All these techniques can of course be used in the present invention. According to a preferred embodiment of the invention, the at least one specific reagent of step b) defined above comprises at least one hybridization probe which is preferably immobilized on a support. This support is preferably a biochip as defined above.

During step c), the determination of the expression of a target gene can be carried out by any of the protocols known to those skilled in the art.

In general, the expression of a target gene can be analysed by detecting the mRNAs (messenger RNAs) that are transcribed from the target gene at a given moment or by detecting the proteins derived from these mRNAs.

The invention relates preferably to the determination of the expression of a target gene by detection of the mRNAs derived from this target gene according to any of the protocols well known to those skilled in the art. According to one specific embodiment of the invention, the expression of several target genes is determined simultaneously, by detection of several different mRNAs, each mRNA being derived from a target gene.

When the specific reagent comprises at least one amplification primer, it is possible, during step c) of the method according to the invention, to determine the expression of a target gene in the following way:

1) After having extracted, as biological material, the total RNAs (comprising the transfer RNAs (tRNAs), the ribosomal RNAs (rRNAs) and the messenger RNAs (mRNAs)) from a biological sample as presented above, a reverse transcription step is carried out in order to obtain the complementary DNAs (or cDNAs) of said mRNAs. By way of indication, this reverse transcription reaction can be carried out using a reverse transcriptase enzyme which makes it possible to obtain, from an RNA fragment, a complementary DNA fragment. The reverse transcriptase enzyme from AMV (Avian Myoblastosis Virus) or from MMLV (Moloney Murine Leukaemia Virus) can in particular be used. When it is more particularly desired to obtain only the cDNAs of the mRNAs, this reverse transcription step is carried out in the presence of nucleotide fragments comprising only thymine bases (polyT), which hybridize by complementarity on the polyA sequence of the mRNAs so as to form a polyT-polyA complex which then serves as a starting point for the reverse transcription reaction carried out by the reverse transcriptase enzyme. cDNAs complementary to the mRNAs derived from a target gene (cDNA specific for the target gene) and cDNAs complementary to the mRNAs derived from genes other than the target gene (cDNA not specific to the target gene) are then obtained.

2) The amplification primer(s) specific for a target gene is (are) brought into contact with the cDNAs specific for the target gene and the cDNAs not specific for the target gene. The amplification primer(s) specific for a target gene hybridize(s) with the cDNAs specific for the target gene and a predetermined region, of known length, of the cDNAs originating from the mRNAs derived from the target gene is specifically amplified. The cDNAs not specific for the target gene are not amplified, whereas a large amount of cDNAs specific for the target gene is then obtained. For the purpose of the present invention, reference is made, without distinction, to “cDNAs specific for the target gene” or to “cDNAs originating from the mRNAs derived from the target gene”. This step can be carried out in particular by means of a PCR-type amplification reaction or by any other amplification technique as defined above. By PCR, it is also possible to simultaneously amplify several different cDNAs, each one being specific for a different target gene, by using several pairs of different amplification primers, each one being specific for a target gene: reference is then made to multiplex amplification.

3) The expression of the target gene is determined by detecting and quantifying the cDNAs specific for the target gene that are obtained during step 2) above. This detection can be carried out after electrophoretic migration of the cDNAs specific for the target gene according to their size. The gel and the medium for migration can include ethydium bromide in order to allow direct detection of the cDNAs specific for the target gene when the gel is placed, after a given migration period, on a UV (ultraviolet)-ray light table, through the emission of a light signal. The greater the amount of cDNAs specific for the target gene, the brighter this light signal. These electrophoresis techniques are well known to those skilled in the art. The cDNAs specific for the target gene can also be detected and quantified using a quantification range obtained by means of an amplification reaction carried out until saturation. In order to take into account the variability in enzymatic effectiveness which may be observed during the various steps (reverse transcription, PCR, etc.), the expression of a target gene of several groups of patients can be normalized by simultaneously determining the expression of a “housekeeping” gene, the expression of which is similar in the various groups of patients. By realizing a ratio of the expression of the target gene to the expression of the housekeeping gene, i.e. by realizing a ratio of the amount of cDNAs specific for the target gene to the amount of cDNAs specific for the housekeeping gene, any variability between the various experiments is thus corrected. Those skilled in the art may refer in particular to the following publications: Bustin S A Journal of molecular endocrinology, 2002, 29: 23-39; Giulietti A Methods, 2001, 25: 386-401.

When the specific reagent comprises at least one hybridization probe, the expression of a target gene can be determined in the following way:

1) After having extracted, as biological material, the total RNAs from a biological sample as presented above, a reverse transcription step is carried out as described above in order to obtain cDNAs complementary to the mRNAs derived from a-target gene (cDNA specific for the target gene) and cDNAs complementary to the mRNAs derived from genes other than the target gene (cDNA not specific for the target gene).

2) All the cDNAs are brought into contact with a support, on which are immobilized capture probes specific for the target gene whose expression it is desired to analyse, in order to carry out a hybridization reaction between the cDNAs specific for the target gene and the capture probes, the cDNAs not specific for the target gene not hybridizing with the capture probes. The hybridization reaction can be carried out on a solid support which includes all the materials as indicated above. According to a preferred embodiment, the hybridization probe is immobilized on a support. Preferably, the support is a biochip. The hybridization reaction can be preceded by a step consisting of enzymatic amplification of the cDNAs specific for the target gene as described above, so as to obtain a large amount of cDNAs specific for the target gene and to increase the probability of a cDNA specific for a target gene hybridizing with a capture probe specific for the target gene. The hybridization reaction can also be preceded by a step consisting in labelling and/or cleaving the cDNAs specific for the target gene as described above, for example using a labelled deoxyribonucleotide triphosphate for the amplification reaction. The cleavage can be carried out in particular by the action of imidazole and manganese chloride. The cDNA specific for the target gene can also be labelled after the amplification step, for example by hybridizing a labelled probe according to the sandwich hybridization technique described in document WO-A-91/19812. Other preferred specific methods for labelling and/or cleaving nucleic acids are described in applications WO 99/65926, WO 01/44507, WO 01/44506, WO 02/090584 and WO 02/090319.

3) A step consisting of detection of the hybridization reaction is subsequently carried out. The detection can be carried out by bringing the support on which the capture probes specific for the target gene are hybridized with the cDNAs specific for the target gene into contact with a “detection” probe labelled with a label, and detecting the signal emitted by the label. When the cDNA specific for the target gene has been labelled beforehand with a label, the signal emitted by the label is detected directly.

When the at least one specific reagent brought into contact during step b) of the method according to the invention comprises at least one hybridization probe, the expression of a target gene can also-be determined in the following way:

1) After having extracted, as biological material, the total RNAs from a biological sample as presented above, a reverse transcription step is carried out as described above in order to obtain the cDNAs of the mRNAs of the biological material. The polymerization of the complementary RNA of the cDNA is subsequently carried out using a T7 polymerase enzyme which functions under the dependency of a promoter and which makes it possible to obtain, from a DNA template, the complementary RNA. The cRNAs of the cDNAs of the mRNAs specific for the target gene (reference is then made to cRNA specific for the target gene) and the cRNAs of the cDNAs of the mRNAs not specific for the target gene are then obtained.

2) All the cRNAs are brought into contact with a support on which are immobilized capture probes specific for the target gene whose expression it is desired to analyse, in order to carry out a hybridization reaction between the cRNAs specific for the target gene and the capture probes, the cRNAs not specific for the target gene not hybridizing with the capture probes. When it is desired to simultaneously analyse the expression of several target genes, several different capture probes can be immobilized on the support, each one being specific for a target gene. The hybridization reaction can also be preceded by a step consisting in labelling and/or cleaving the cRNAs specific for the target gene, as described above.

3) A step consisting of detection of the hybridization reaction is subsequently carried out. The detection can be carried out by bringing the support on which the capture probes specific for the target gene are hybridized with the cRNA specific for the target gene into contact with a “detection” probe labelled with a label, and detecting the signal emitted by the label. When the cRNA specific for the target gene has been labelled beforehand with a label, the signal emitted by the label is detected directly. The use of cRNA is particularly advantageous when a support of biochip type on which a large number of probes are hybridized is used.

The analysis of the expression of a target gene chosen from any one of SEQ ID Nos. 1 to 37 therefore makes it possible to provide a tool for neuroblastoma prognosis. It is possible, for example, to analyse the expression of a target gene in a patient whose prognosis is not known, and to compare this with known values of mean expression of the target gene from patients with a good prognosis and known values of mean expression of the target gene from patients with a poor prognosis. This makes it possible to determine whether the patient has a good or a poor prognosis in order to provide the patient with a suitable treatment.

According to a preferred embodiment of the invention, during step b), the biological material is brought into contact with at least 37 specific reagents chosen from the reagents specific for the target genes exhibiting a nucleic acid sequence having any one of SEQ ID Nos. 1 to 37, and, during step C, the expression of at least 37 of said target genes is determined.

According to another preferred embodiment, during step b), the biological material is brought into contact with at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35 or at least 36 specific reagents chosen from the reagents specific for the target genes exhibiting a nucleic acid sequence having any one of SEQ ID Nos. 1 to 37, and the expression of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35 or at least 36 of said target genes is determined during step c.

According to another preferred embodiment, during step b), the biological material is brought into contact with at least 19 specific reagents chosen from the reagents specific for the target genes exhibiting a nucleic acid sequence having SEQ ID No. 1; SEQ ID No. 2; SEQ ID No. 3; SEQ ID No. 7; SEQ ID No. 8; SEQ ID No. 9; SEQ ID No. 10; SEQ ID No. 14; SEQ ID No. 16; SEQ ID No. 20; SEQ ID No. 21; SEQ ID No. 22; SEQ ID No. 25; SEQ ID No. 27; SEQ ID No. 29; SEQ ID No. 31; SEQ ID No. 34; SEQ ID No. 36 or SEQ ID No. 37, and, during step c), the expression of at least 19 of said target genes is determined.

According to another preferred embodiment, during step b), the biological material is brought into contact with at least 2, at least 3, at least 4, at least 5, at least 6, at least 7, at least 8, at least 9, at least 10, at least 11, at least 12, at least 13, at least 14, at least 15, at least 16, at least 17, at least 18 or at least 19 specific reagents chosen from the reagents specific for the target genes exhibiting a nucleic acid sequence having SEQ ID No. 1; SEQ ID No. 2; SEQ ID No. 3; SEQ ID No. 7; SEQ ID No. 8; SEQ ID No. 9; SEQ ID No. 10; SEQ ID No. 14; SEQ ID No. 16; SEQ ID No. 20; SEQ ID No. 21; SEQ ID No. 22; SEQ ID No. 25; SEQ ID No. 27; SEQ ID No. 29; SEQ ID No. 31; SEQ ID No. 34; SEQ ID No. 36 or SEQ ID No. 37, and, during step c), the expression of at least 2, at least 3, at least 4, at least 5, at least 6, at least 7, at least 8, at least 9, at least 10, at least 11, at least 12, at least 13, at least 14, at least 15, at least 16, at least 17, at least 18 or at least 19 of said target genes is determined.

According to another preferred embodiment, during step b), the biological material is brought into contact with at least 16 specific reagents chosen from the reagents specific for the target genes exhibiting a nucleic acid sequence having SEQ ID No. 1; SEQ ID No. 2; SEQ ID No. 3; SEQ ID No. 7; SEQ ID No. 8; SEQ ID No. 9; SEQ ID No. 10; SEQ ID No. 20; SEQ ID No. 21; SEQ ID No. 22; SEQ ID No. 25; SEQ ID No. 29; SEQ ID No. 31; SEQ ID No. 34; SEQ ID No. 36 or SEQ ID No. 37, and the expression of at least 16 of said target genes is determined during step c).

According to another preferred embodiment, during step b), the biological material is brought into contact with at least 12 specific reagents chosen from the reagents specific for the target genes exhibiting a nucleic acid sequence having SEQ ID No. 2; SEQ ID No. 3; SEQ ID No. 7; SEQ ID No. 8; SEQ ID No. 10; SEQ ID No. 20; SEQ ID No. 22; SEQ ID No. 25; SEQ ID No. 29; SEQ ID No. 31; SEQ ID No. 34 or SEQ ID No. 37, and, during step c), the expression of at least 12 of said target genes is determined.

According to another preferred embodiment, during step b), the biological material is brought into contact with at least 9 specific reagents chosen from the reagents specific for the target genes exhibiting a nucleic acid sequence having SEQ ID No. 2; SEQ ID No. 3; SEQ ID No. 7; SEQ ID No. 8; SEQ ID No. 10; SEQ ID No. 22; SEQ ID No. 25; SEQ ID No. 29 or SEQ ID No. 34, and, during step c), the expression of at least 9 of said target genes is determined.

The use of a restricted panel of genes is particularly suitable for obtaining a prognostic tool. This is because the analysis of the expression of about ten genes does not require the customized fabrication of DNA chips, and can be carried out directly by PCR or NASBA techniques, which provides for considerable economical asset and simplified implementation.

The attached figures are given by way of explanatory examples and are in no way limiting in nature. They will make it possible to understand the invention more fully.

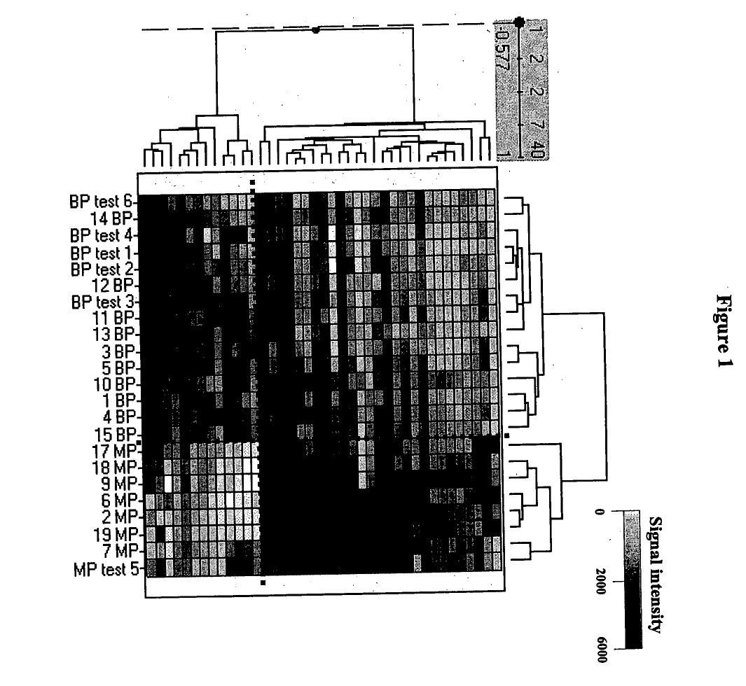

FIG. 1 shows a dendogram obtained from 23 samples of tumours derived from patients with a good prognosis (GP) or with a poor prognosis (PP), and the use of a panel of 40 probes for analyzing the expression of the 37 genes presented above in Table 1. On this dendogram, there are 23 columns corresponding to the 23 tumour samples, and 40 lines corresponding to the 40 probes used for analyzing the expression of the 37 genes. The tumour samples, and also the genes having a comparable expression profile, demonstrated by a Pearson-type correlation, have been placed side by side. The tumour samples were classified according to the non-weighted mean method (Spotfire Decision Site for Functional Genomics V7.1, manual), whereas the genes were classified according to their mean value of expression obtained in all the samples. The expression level of each gene, calculated using the Microarray Suite software (MAS5.0, Affymetrix), is represented by various levels of colour. Thus, the white colour corresponds to a low expression level, the grey colour corresponds to an intermediate expression level, whereas the black colour corresponds to a high expression level. The length of the branches of the dendogram is correlated with the expression profile and the dash line which divides the dendogram makes it possible to distinguish two groups of patients: a first group of patients with a poor prognosis “PP” and a second group of patients with a good prognosis “GP”. The six “PP-test” and “GP-test” tumours are tumours which were analysed “blind”, i.e. without knowing their prognosis.

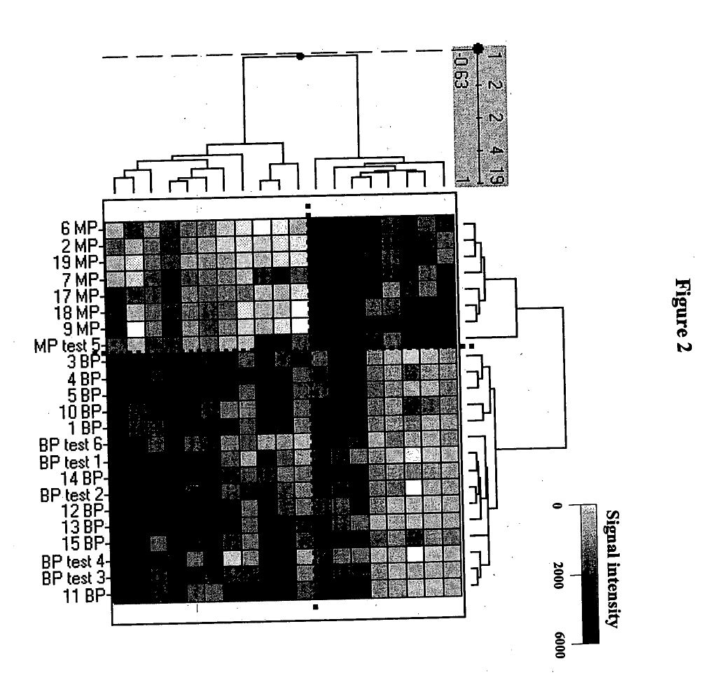

FIG. 2 shows a dendogram obtained from 23 samples of tumours derived from patients with a good prognosis or with a poor prognosis, and the analysis of the expression of 19 genes. This dendogram was obtained in a manner comparable to that described for FIG. 1.

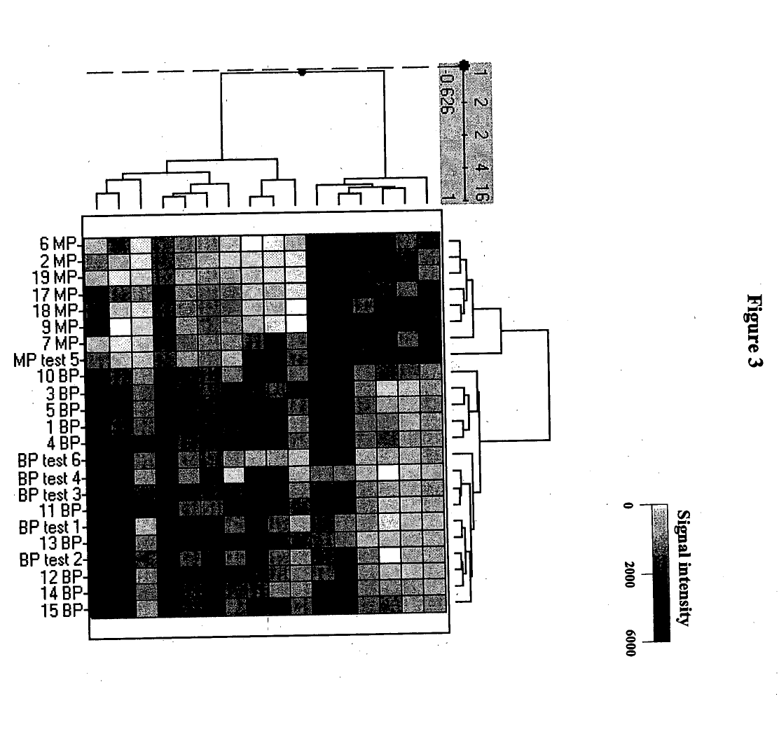

FIG. 3 shows a dendogram obtained from 23 samples of tumours derived from patients with a good prognosis or with a poor prognosis, and the analysis of the expression of 16 genes. This dendograin was obtained in a manner comparable to that described for FIG. 1.

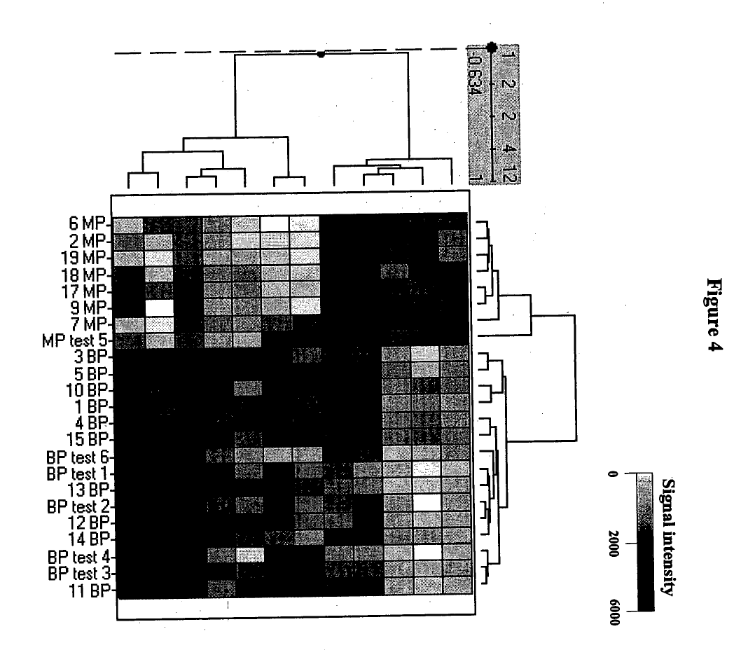

FIG. 4 shows a dendogram obtained from 23 samples of tumours derived from patients with a good prognosis or with a poor prognosis, and the analysis of the expression of 12 genes. This dendogram was obtained in a manner comparable to that described for FIG. 1.

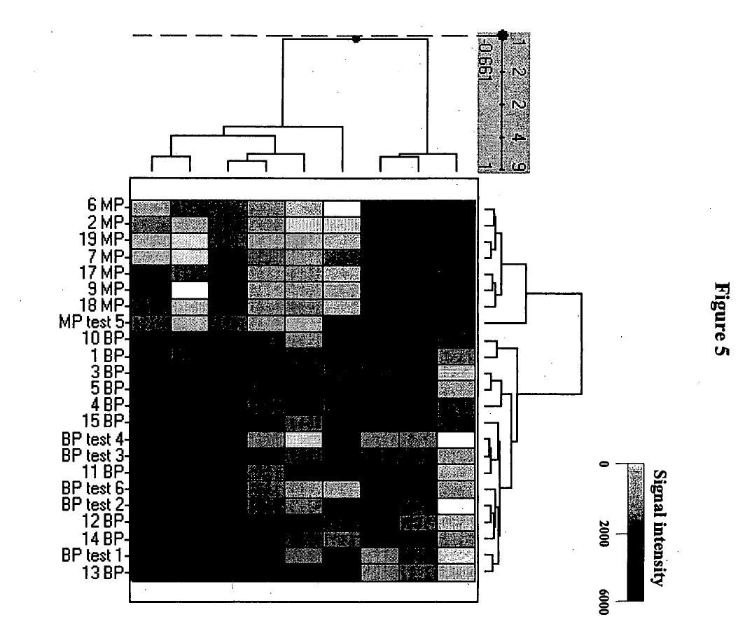

FIG. 5 shows a dendogram obtained from 23 samples of tumours derived from patients with a good prognosis or with a poor prognosis, and the analysis of the expression of 9 genes. This dendogram was obtained in a manner comparable to that described for FIG. 1.

The following examples are given by way of illustration and are in no way limiting in nature. They will make it possible to understand the invention more fully.

### EXAMPLE 1

### Search for an Expression Profile for Neuroblastoma Prognosis

Characteristics of the biological samples (localized tumours or bone marrow punctures): 23 neuroblastoma samples, obtained from the Centre Leon Berard (CLB) in Lyons, France, were used in this study. These neuroblastoma samples were taken prior to any therapeutic treatment. Each tumour was classified according to the INSS classification (International Neuroblastoma Staging System; Brodeur et al; (1993) J. Clin. Oncol. 11, 1466-77). 12 stage 1/2 tumours, 4 stage 4s tumours and 7 stage 4 samples were then distinguished (2 tumour punctures, 1 biopsy, 4 marrow punctures massively invaded). The histochemical analysis showed, in the localized tumours, the presence of approximately 80% of tumour cells. The immunocytochemical analysis also showed in the bone marrow punctures the presence of approximately 80% of tumour cells. The median age of the patients at the time of the neuroblastoma diagnosis was 10 and a half months, and 5 patients died during the median follow-up period of 75 months. The patients who died during the study and the patients with a stage IV neuroblastoma were described as patients with a poor prognosis (PP), while the patients alive and having developed a stage 1, 2 and 4s neuroblastoma were described as patients with a good prognosis (GP) (description according to Brodeur, 2003, Nat Rev Cancer, 203-216). This analysis was thus carried out on 8 PP patients and 15 GP patients.

Extraction of the biological material (total RNAs) from the biological sample: The total RNAs were extracted from each tumour or bone marrow puncture according to a protocol well known to those skilled in the art (see in particular Ausubel et al (1997), Current protocols in Molecular Biology, Volume 1, John Wiley and Sons, New York). For this, each biological sample was homogenized in 1 ml of Trizol (Invitrogen, Cergy Pointoise, France), and treated with 300 μl of chloroform in order to eliminate any protein and lipophilic contaminants. The total RNAs were subsequently precipitated with 750 μl of isopropanol, washed twice with an 80% (vol/vol) ethanol solution and redissolved in DEPC water. The total RNAs were subsequently purified on a Qiagen RNeasy column (Qiagen, Hilden, Germany) in accordance with the manufacturer's instructions, with the exception of the final elution, which was carried out in 200 μl of RNAse-free water after 1 min of incubation at 65° C. Prior to the reverse transcription step, a step consisting of precipitation with ammonium acetate (0.5 vol, 7.5M) and ethanol (2.5 vol) was carried out in order to guarantee the purification of the total RNAs. The quality of the total RNAs was analysed using the Agilent 2100 bioanalyser (Agilent Technologies, Waldbronn, Germany). The total RNAs comprise the transfer RNAs, the messenger RNAs (mRNAs) and the ribosomal RNAs.

Synthesis of cDNAs, obtaining of cRNAs and labelling of cRNAs, and quantification: In order to analyse the expression of the target genes according to the invention, the complementary DNAs (cDNAs) of the mRNAs contained in the total RNAs as purified above were obtained from 10 μg of total RNAs using 400 units of the SuperScriptII reverse transcription enzyme (Invitrogen) and 100 pmol of poly-T primer containing the T7 promoter (T7-oligo(dT)24-primer, Proligo, Paris, France). The cDNAs thus obtained were subsequently extracted with phenol/chloroform and precipitated as described above with ammonium acetate and ethanol, and redissolved in 24 μl of DEPC water. A 20 μl volume of this purified cDNA solution was subsequently subjected to an in vitro transcription using a T7 RNA polymerase which specifically recognizes the T7 polymerase promoter as mentioned above. This transcription makes it possible to obtain the cRNA of the cDNA. This transcription was carried out using a Bioarray High Yield RNA Transcript Labeling Kit (Enzo Diagnostics, Farmingdale, N.Y.), which makes it possible not only to obtain the cRNA, but also allows the incorporation of biotinylated cytidine and uridine bases during the synthesis of the cRNA.

The purified cRNAs were subsequently quantified by spectrophotometry, and the cRNA solution was adjusted to a concentration of 1 μg/μl of cRNA. The step consisting of cleavage of these cRNAs was subsequently carried out at 94° C. for 35 min, using a fragmentation buffer (40 mM of trisacetate, pH 8.1, 100 mM of potassium acetate, 30 mM of magnesium acetate) in order to bring about hydrolysis of the cRNAs and to obtain fragments of 35 to 200 bp. The success of such fragmentation was verified by means of a 1.5% agarose gel electrophoresis.

Demonstration of a Differential Expression Profile Between GP and PP Patients

The expression of approximately 10 000 genes was analysed and compared between the GP and PP patients. For this, 10 μg of fragmented cRNAs derived from each sample were added to a hybridization buffer (Affymetrix) and 200 μl of this solution were brought into contact for 16 h at 45° C. on an expression chip (Human Genome U95Av2 GeneChip® (Affymetrix)), which comprises 12 625 groups of probes representing approximately 10 000 genes, according to the Affymetrix protocol as described on the Affymetrix internet site (see, in particular, at the following address: http://www.affymetrix.com/support/downloads/manuals/expression_s2_manual.pdf). In order to record the best hybridization and washing performance levels, biotinylated RNAs described as “control” (bioB, bioC, bioD and cre) and oligonucleotides (oligo B2) were also included in the hybridization buffer. After the hybridization step, the biotinylated cRNA solution hybridized on the chip was revealed using a solution of streptavidin-phycoeryrin and the signal was amplified using an anti-streptavidin antibody. The hybridization was carried out in a “GeneChip Hybridization oven” (Affymetrix), and the Euk GE-WS2 protocol of the Affymetrix protocol was followed. The washing and visualizing steps were carried out on a “Fluidics Station 400” (Affymetrix). Each U95Av2 chip was subsequently analysed on an Agilent G2500A GeneArray Scanner at a resolution of 3 microns in order to pinpoint the zones hybridized on the chip. This scanner makes it possible to detect the signal emitted by the fluorescent molecules after excitation with an argon laser using the epifluorescence microscope technique. For each position, a signal proportional to the amount of cRNAs attached is thus obtained. The signal was subsequently analysed using the Microarray Suite 5.0 software (MAS5.0, Affymetrix).

In order to prevent the variations obtained by using various chips, an overall normalization approach has been taken using the MAS5.0 software (Affymetrix), which makes it possible to convert the raw data obtained for each chip into an average signal within an intensity of 500. The results obtained on one chip can then be compared with the results obtained on another chip. The MAS5.0 software also made it possible to include a statistical algorithm in order to consider whether or not a gene was expressed. Each gene represented on the U95Av2 chip was covered with 16 to 20 pairs of probes of 25 oligonucleotides. The “term pair of probes” is intended to mean a first probe which hybridized perfectly (reference is then made to PM, or perfect match, probes) with one of the cRNAs derived from a target gene, and a second-probe, identical to the first probe with the exception of a mismatch (reference is then made to an MM, or mismatched, probe) at the centre of the probe. Each MM probe was used to estimate the background noise corresponding to a hybridization between two nucleotide fragments of noncomplementary sequence (Affymetrix technical note “Statistical Algorithms Reference Guide”; Lipshutz, et al (1999) Nat. Genet. 1 Suppl., 20-24). Two stage IV tumours exhibiting a low percentage of genes expressed, due to a bias either in the quality of the cRNAs or in the hybridization step, were excluded from the analysis. The remaining 23 samples showed an average of 48% of genes expressed.

The expression data analysis was carried out using the Microsoft Excel software, the Spotfire Decision Site for Functional Genomics V7.1 software (Spotfire A B, Gothenburg, Sweden) and the PAM (Prediction Analysis in Microarrays) module of the R statistics software (Ihaka & Gentleman (1996) Journal of Computational and Graphical Statistics 5, 299-314; Tibshirani, et al (2002) Proc. Natl. Acad. Sci. 99, 6567-6572).

From the 12625 groups of probes, representing approximately 10 000 genes, of the chip, the inventors selected the relevant genes which were correlated with a poor neuroblastoma prognosis.

For this, a first step consisted in excluding the genes exhibiting an expression level that is comparable between all the groups of patients [Tibshirani, et al Proc. Natl. Acad. Sci. 99, 6567-6572]. The genes that were nonexpressed in all the patients were also excluded (MAS5.0 software). Finally, some genes were excluded if the expression mean of the 2 groups (patients with a good prognosis and patients with a poor prognosis) was less than 500 or if the ratio of the expression means between the patients with a poor prognosis and the patients with a good prognosis was between 0.7 and 1.3.

The expression of the remaining 1488 genes was subsequently analysed (PAM algorithm, Tibshirani, R., Hastie, T., Narasimhan, B. and Chu, G. (2002) Diagnosis of multiple cancer types by shrunken centroids of gene expression. Proc. Natl. Acad. Sci. 99, 6567-6572).

Results obtained: Firstly, 37 genes making it possible to differentiate the patients with good and poor prognosis were identified. The increase or the decrease in expression of each of these genes, observed in the patients with a poor prognosis compared with the patients with a good prognosis, is indicated in Table 2.

TABLE 2
List of the 37 genes differentially expressed in the GP and PP patient neuroblastomas

| SEQ ID No. | Sequence description | Genbank No. | Expression PP vs GP |
|---|---|---|---|
| 1 | Flap structure-specific endonuclease 1 | NM_004111 | increased |
| 2 | Ubiquitin-conjugating enzyme E2C | NM_007019 | increased |
| 3 | Insulin-like growth factor binding protein | NM_001553 | decreased |
| | 7(MAC25) | | |
| 4 | Collagen type I, alpha 2 chain | NM_000089 | decreased |
| 5 | Nucleolin | NM_005381 | increased |
| 6 | Interleukin enhancer binding factor 3 | NM_004516 | increased |
| 7 | cDNA FLJ30781 fis, clone FEBRA2000874, | AK055343/ | decreased |
| | corresponding to the gene encoding adenylate | NM_021116 | |
| | cyclase 1 (brain) | | |
| 8 | TIF1beta zinc finger protein | X97548 | increased |
| 9 | Likely orthologue of mouse tumour | NM_020755 | decreased |
| | differentially expressed 1 TDE1L | | |
| 10 | DKFZp434D1112_s1 434 (synonym: htes3) cDNA | AL039831 | decreased |
| | clone DKFZp434D1112 3′ | | |
| 11 | N-MYC proto-oncogene | NM_005378 | increased |
| 12 | Small nuclear ribonucleoprotein D2 | NM_004597 | increased |
| | polypeptide 16.5 kDa | | |
| 13 | DNA replication licensing factor MCM2 | NM_004526 | increased |
| 14 | RuvB-like DNA helicase TIP49b | NM_006666 | increased |
| 15 | Immediate early protein ETR101 | NM_004907 | increased |
| 16 | RNA binding protein S1, serine-rich domain | NM_006711 | increased |
| 17 | Ornithine decarboxylase 1 | NM_002539 | increased |
| 18 | Activity-related cytoskeleton-asso. protein | NM_015193 | increased |
| | (KIAA0278) | | |
| 19 | Secretogranin II (chromogranin C) | NM_003469 | decreased |
| 20 | Structure specific recognition protein 1 | NM_003146 | increased |
| 21 | Collagen type VI, alpha 3 chain | NM_004369 | decreased |
| 22 | Small nuclear ribonucleoprotein polypeptides B | NM_003091 | increased |
| | and B1 | | |
| 23 | Acidic nuclear phosphoprotein 32 family, | NM_006401 | increased |
| | member B | | |
| 24 | Non-POU domain containing, octamer-binding | NM_007363 | increased |
| 25 | Peripheral myelin protein 22 | NM_000304 | decreased |
| 26 | Small nuclear ribonucleoprotein polypeptide E | NM_003094 | increased |
| 27 | Putative L-type neutral amino acid transporter | AB007896 | decreased |
| | (KIAA0436) | | |
| 28 | Fibrillarin | NM_001436 | increased |
| 29 | Tripartite motif-containing 2 | NM_015271 | decreased |
| 30 | DNA replication licensing factor MCM6 | NM_005915 | increased |
| 31 | Polypyrimidine tract binding protein 1 | NM_002819 | increased |
| 32 | Small nuclear ribonucleoprotein polypeptide A | NM_004596 | increased |
| 33 | Creatine kinase, brain | NM_001823 | increased |
| 34 | Erythrocyte membrane protein band 4.1 like3 | NM_012307 | decreased |
| 35 | Hypothetical protein MGC3077 | NM_024051 | increased |
| 36 | Tissue alpha-L-fucosidase 1 | NM_000147 | decreased |
| 37 | Secreted protein acidic and rich in cysteine | NM_003118 | decreased |

These results were also validated using another molecular biology technique in which the analysis of the gene expression as presented in Table 2 was carried out by RT-PCR.

For this, a reverse transcription (RT) reaction was carried out using 1 μg of total RNA as obtained above (First strand cDNA synthesis kit, Amersham). The reverse transcription was carried out for 1 h at 37° C. Each cDNA solution was diluted 6-fold before the PCR was carried out.

The expression of the mRNAs of genes of Table 2 (Peripheral myelin protein or PMP22 (SEQ ID No. 25); Insulin-like growth factor binding protein or IGFBP7 (SEQ ID No. 3; SPARC (SEQ ID No. 37); EPB41L3 (SEQ ID No. 34)) was subsequently analysed by PCR (polymerase chain reaction) and the use of specific amplification primers (amplification of the PMP22 gene: sense strand: 5′-AGGGAGGAAGGGAAAACAGA-3′ (SEQ ID No. 38); antisense strand: 5′-TTAAGGCTCAACACGAGGCT-3′ (SEQ ID No. 39); IGFBP7 gene: sense strand: 5′-CTTGAGCTGTGAGGTCATCG-3′ (SEQ ID No. 40); antisense strand: 5′-TATAGCTCGGCACCTTCACC-3′ (SEQ ID No. 41); SPARC gene: sense strand: 5′-CTGCCTGCCACTGAGGGTTCC-3′ (SEQ ID No. 42); antisense strand: 5′-TCCAGGCAGAACAACAAACCATCC-3′ (SEQ ID No. 43); EPB41L3 gene: sense strand: 5′-ACCACCACCACTACCCACAT-3′ (SEQ ID No. 44); antisense strand: 5′-TGGTTTTCCTAACGGTTTGC-3′ (SEQ ID No. 45); beta-actin gene: sense strand: 5′-TGTTGGCGTACAGGTCTTTGC-3′ (SEQ ID No. 46); antisense strand: 5′-GCTACGAGCTGCCTGACGG-3′ (SEQ ID No. 47)). The expression of the gene encoding β-actin was used as a control. Thirty PCR cycles were subsequently carried out in the presence of the various amplification primers (0.2 μM); of dNTPs (0.15 mM, Euromedex) and of polymerase enzyme (Taq Polymerase; 0.027 U/μl; Perkin Elmer) (denaturation 30 sec at 94° C., hybridization 1 min at 60° C.; polymerization 1 min at 72° C.).

The results obtained are given in Table 3 below, which shows the correlation that exists between the results obtained using a biochip and those obtained by RT-PCR.

TABLE 3

| | GP patients | | PP patients | | |
|---|---|---|---|---|---|
| | Biochip | RT-PCR | Biochip | RT-PCR | |
| | results | results | results | results | p |
| SEQ ID No. 37: | 3498 | 2.2 | 709 | 0.87 | 0.04 |
| SPARC | | | | | |
| SEQ ID No. 3 | 4112 | 3.7 | 691 | 1.87 | 0.003 |
| IGFBP7 | | | | | |
| SEQ ID No. 34 | 6041 | 4.2 | 986 | 1.5 | 0.001 |
| EPB41L3 | | | | | |
| SEQ ID No. 25 | 9051 | 3.8 | 2282 | 2.75 | 0.003 |
| PMP22 | | | | | |

The RT-PCR results, obtained from 15 GP patients and 8 PP patients, are expressed by the ratio of relative quantification between the mRNAs of the target gene and the mRNAs of the β-actin gene which was used as a control. The results are expressed in terms of the mean of the ratios obtained for each of the groups of patients. The correlation of the results obtained, firstly, with the biochip and, secondly, with the RT-PCR technique was established by means of Kendall's Tau-B correlation test. The PP patients exhibited a decreased expression level for the SPARC, IGFBP7, EPB41L3 and PMP22 genes, confirming the results given in Table 2.

The expression of the mRNAs of the genes of SEQ ID No. 37: SPARC; SEQ ID No. 2: UBE2C; SEQ ID No. 3: IGFBP7; SEQ ID No. 8: TRIM28; SEQ ID No. 22: SNRBP; SEQ ID No. 25: PMP22; SEQ ID No. 29: TRIM2; SEQ ID No. 34: EPB41L3; SEQ ID No. 7: clone FEBRA2000874 was also analysed by quantitative RT-PCR.

The cDNAs required for the analysis of each of these target genes were obtained from one microgram of total RNAs (first-strand DNA synthesis kit, Amersham, France). After a 6-fold dilution, 2.5 μl of cDNA were used in real-time PCR, in the presence of a pair of primers (300 nM) specific for each target gene (cf. table below) and SYBR-Green Master Mix buffer.

| SEQ ID | | |
|---|---|---|
| AMPLIFIED | Sense primer | Antisense primer |
| SEQ ID No.37: | SEQ ID No.48: | SEQ ID No.49: |
| SPARC | 5′-CACATTAGGCTGTTGGTTCA AACT-3′ | 5′-CAGGATGCGCTGACCACTT-3′ |
| SEQ ID No.2: | SEQ ID No.50: | SEQ ID No.51: |
| UBE2C | 5′-TCCTCACGCCCTGCTATCA-3′ | 5′-TTCAGGATGTCCAGGCATAT GT-3′ |
| SEQ ID No.3: | SEQ ID No.52: | SEQ ID No.53: |
| IGFBP7 | 5′-TGTCCTCATCTGGAACAAGG-3′ | 5′-GGCAGGAGTTCTGTCCTTTG-3′ |
| SEQ ID No.7: | SEQ ID No.54: | SEQ ID No.55: |
| clone | 5′-TTTACATCCAGAGGCACGAC-3′ | 5′-CACGATGTCAGCAAACAGG-3′ |
| FEBRA2000874 | | |
| SEQ ID No.8: | SEQ ID No.56: | SEQ ID No.57: |
| TRIM28 | 5′-CAGGAAGGCTATGGCTTTGG-3′ | 5′-CCGTTTCACACCTGACACAT G-3′ |
| SEQ ID No.22: | SEQ ID No.58: | SEQ ID No.59: |
| SNRBP | 5′-GCTGGACCGGAAGTAGGTTT CT-3′ | 5′-GCCGCTACCGGAAATGC-3′ |
| SEQ ID No.25: | SEQ ID No.60: | SEQ ID No.61: |
| PMP22 | 5′-GACCCAGTGCATCCAACAG A-3′ | 5′-GTGTGCGCGTAAAGCTTCAC-3′ |
| SEQ ID No.29: | SEQ ID No.62: | SEQ ID No.63: |
| TRIM2 | 5′-CAGTAACAACCAATGTGTGC AG-3′ | 5′-TGCCAAAACGACTTTTGAAC-3′ |
| SEQ ID No.34: | SEQ ID No.64: | SEQ ID No.65: |
| EPB41L3 | 5′-GTTGGACCCTGCTAAGGAAA-3′ | 5′-CAGATAGTTGGGCAGGGTCT-3′ |
| Housekeeping | SEQ ID No.66: | SEQ ID No.67: |
| gene HIPRT1 | 5′-CACTGGCAAAACAATGCAGA CT-3′ | 5′-CGACCTTGACCATCTTTGGA TT-3′ |

The total reaction volume was 15 μl. The PCR amplification was carried out in 96-well microplates, using the ABI Prism 7000 Sequence Detection system (Applied BioSystem USA). The HPRT1 reference gene and the target gene were analysed simultaneously. After denaturation at 95° C. for 10 min, the amplification was carried out under the following conditions: 40 cycles of 15 seconds at 95° C. followed by 1 minute at 60° C. The experiments were carried out in duplicate. The quantification was carried out using the standard curve method and using the CT comparative method as recommended by the manufacturer. The standard curves were obtained using dilutions of cDNA from neuroblastoma cell lines, and were prepared for each PCR. The expression of the target gene was determined using these standard curves. The relative expression of each target gene was defined by comparison with the expression of the reference gene. The Pearson and Spearman correlation tests were used to calculate the correlation between the results obtained on a chip and the results obtained by RT-PCR.

The results are given in the table below.

| | Spearman test | Pearson test |
|---|---|---|
| SEQ ID N0. 37: SPARC | p = 0.0008 | p = 0.0163 |
| SEQ ID No. 2: UBE2C | p < 0.0001 | p < 0.0001 |
| SEQ ID No. 3: IGFBP7 | p = 0.004 | p = 0.0977 |
| SEQ ID No. 7 | p < 0.0001 | p = 0.001 |
| SEQ ID No. 8: TRIM28 | p = 0.0428 | p = 0.0058 |
| SEQ ID No. 22: SNRBP | p = 0.0664 | p = 0.503 |
| SEQ ID No. 25: PMP22 | p = 0.021 | p = 0.0079 |
| SEQ ID No. 29: TRIM2 | p = 0.2983 | p = 0.232 |
| SEQ ID No. 34: EPB41L3 | p = 0.0123 | p = 0.0606 |

These results showed a good correlation (p<0.05) between the results obtained on a chip and by RT-PCR, concerning in particular the genes of SEQ ID No. 2, 7, 8 and 25, suggesting that these 4 genes are particularly relevant for neuroblastoma prognosis.

The inventors also studied the simultaneous expression of the 37 genes of Table 2 in order to obtain an expression profile. The results are given in FIG. 1. On this dendogram, two groups having two different expression profiles are observed: a first group that makes it possible to classify the patients with a good prognosis (GP) and a second group that makes it possible to classify the patients with a poor prognosis (PP).

With the objective of validating the discriminatory capacity of the expression profile of these 37 genes, 6 additional tumours from “test” patients were analysed without prior knowledge of their prognosis, and classified as being of good prognosis, “GP-test”, or of poor prognosis, “PP-test”, according to the analysis of their expression profile. Their correct classification was subsequently verified according to their clinical properties: all the “test” samples analysed blind by analysing the expression of 37 genes had been correctly classified in the group of patients with a poor prognosis, “PP-test”, or in the group of patients with a good prognosis “GP-test”. This confirms that the analysis of the expression of these 37 genes is a good tool for neuroblastoma prognosis.

By way of indication, the N-myc oncogene was also used as a prognostic tool. The use of this gene revealed 5 patients with a poor prognosis (PP). However, 3 patients also had a poor prognosis although no increase in the expression of the N-myc oncogene was observed, suggesting that the analysis of this gene only is not sufficient for neuroblastoma prognosis.

The inventors also defined more restricted panels of genes that also make it possible to discriminate between patients having a good prognosis and patients having a poor prognosis.

A first panel comprised 19 genes, which are presented in Table 4. The results are expressed in terms of the ratio obtained between the mean expression of the gene in PP patients and the expression of the gene in GP patients (PP/GP ratio).

TABLE 4
List of the 19 genes differentially expressed
in the neuroblastomas of GP and PP patients

| SEQ ID No. | Sequence description | Genbank No. | PP/GP ratio |
|---|---|---|---|
| 1 | Flap structure-specific endonuclease 1 (FEN1) | NM_004111 | 2.7 |
| 2 | Ubiquitin-conjugating enzyme E2C (UBE2C) | NM_007019 | 2.9 |
| 3 | Insulin-like growth factor binding protein 7 | NM_001553 | 0.2 |
| | (IGFBP7) | | |
| 7 | cDNA FLJ30781 fis, clone FEBRA2000874, | AK055343/ | 0.3 |
| | corresponding to the gene encoding adenylate | NM_021116 | |
| | cyclase 1 (brain) | | |
| 8 | TIF1beta zinc finger protein | X97548 | 1.8 |
| 9 | Likely orthologue of mouse tumour | NM_020755 | 0.5 |
| | differentially expressed 1, like (TDE1L) | | |
| 10 | DKFZp434D1112_s1 434 (synonym: htes3) | AL039831 | 0.4 |
| | cDNA clone DKFZp434D1112 3′ | | |
| 14 | RuvB-like 2 (E. coli)(RUVBL2) | NM_006666 | 2.1 |
| 16 | RNA binding protein S1, serine-rich domain | NM_006711 | 1.6 |
| | (RNPS1), transcript variant 1 | | |
| 20 | Structure specific recognition protein 1 | NM_003146 | 2.0 |
| | (SSRP1) | | |
| 21 | Collagen, type VI, alpha 3 (COL6A3), | NM_004369 | 0.2 |
| | transcript variant 1 | | |
| 22 | Small nuclear ribonucleoprotein polypeptides | NM_003091 | 1.7 |
| | B and B1 (SNRPB) | | |
| 25 | Peripheral myelin protein 22 (PMP22), | NM_000304 | 0.3 |
| | transcript variant 1 | | |
| 27 | KIAA0436 mRNA, partial cds | AB007896 | 0.5 |
| 29 | Tripartite motif-containing 2 (TRIM2) | NM_015271 | 0.4 |
| 31 | Polypyrimidine tract binding protein 1 | NM_002819 | 2.1 |
| | (PTBP 1), transcript variant 1 | | |
| 34 | Erythrocyte membrane protein band 4.1-like 3 | NM_012307 | 0.2 |
| 36 | Fucosidase, alpha-L- 1, tissue (FUCA1) | NM_000147 | 0.2 |
| 37 | Secreted protein, acidic, cysteine-rich | NM_003118 | 0.2 |
| | (osteonectin) (SPARC) | | |

The inventors also studied the simultaneous expression of these 19 genes in order to obtain an expression profile. The results are given in FIG. 2. On this dendogram, two groups having two different expression profiles are observed: a first group that makes it possible to classify the patients having a good prognosis (GP) and a second group that makes it possible to classify the patients having a poor prognosis (PP).

With the objective of validating the discriminatory capacity of these 19 genes, 6 tumours from “test” patients were analysed without prior knowledge of their prognosis. Thus, the six “PP-test” and “GP-test” tumours presented in FIG. 3 are tumours which were analysed “blind”. Their correct classification was verified according to their clinical property: all the “PP-test” patients classified according to their expression profile as patients with a poor prognosis proved to be patients with a poor prognosis, and all the “GP-test” patients classified according to their expression profile as patients with a good prognosis proved to be patients with-a good prognosis. In a comparable manner, a second panel comprised 16 genes as presented in Table 5.

TABLE 5
List of the 16 genes expressed differentially
in the neuroblastomas of GP and PP patients

| SEQ | | |
|---|---|---|
| ID | | Genbank |
| No. | Sequence description | No. |
| 1 | Flap structure-specific endonuclease 1 | NM_004111 |
| | (FEN1) | |
| 2 | Ubiquitin-conjugating enzyme E2C | NM_007019 |
| | (UBE2C) | |
| 3 | Insulin-like growth factor binding | NM_001553 |
| | protein 7 (IGFBP7) | |
| 7 | cDNA FLJ30781 fis, clone FEBRA2000874, | AK055343/ |
| | corresponding to the gene encoding | NM_021116 |
| | adenylate cyclase 1 (brain) | |
| 8 | TIF1beta zinc finger protein | X97548 |
| 9 | Likely orthologue of mouse tumour | NM_020755 |
| | differentially expressed 1, like | |
| | (TDE1L) | |
| 10 | DKFZp434D1112_s1 434 (synonym: htes3) | AL039831 |
| | cDNA clone DKFZp434D1112 3′ | |
| 20 | Structure specific recognition protein | MM_003146 |
| | 1 (SSRP1) | |
| 21 | Collagen, type VI, alpha 3 (COL6A3), | NM_004369 |
| | transcript variant 1 | |
| 22 | Small nuclear ribonucleoprotein | NM_003091 |
| | polypeptides B and B1 (SNRPB) | |
| 25 | Peripheral myelin protein 22 (PMP22), | NM_000304 |
| | transcript variant 1 | |
| 29 | Tripartite motif-containing 2 (TRIM2) | NM_015271 |
| 31 | Polypyrimidine tract binding protein 1 | NM_002819 |
| | (PTBP1), transcript variant 1 | |
| 34 | Erythrocyte membrane protein band | NM_012307 |
| | 4.1-like 3 (EPB41L3) | |
| 36 | Fucosidase, alpha-L- 1, tissue (FUCA1) | NM_000147 |
| 37 | Secreted protein, acidic, cysteine- | NM_003118 |
| | rich (osteonectin) (SPARC) | |

The inventors also studied the simultaneous expression of these 16 genes in order to obtain an expression profile. The results are given in FIG. 3. On this dendogram, two groups having two different expression profiles are observed: a first group that makes it possible to classify the patients having a good prognosis (GP) and a second group that makes it possible to classify the patients having a poor prognosis (PP).

A third panel comprised 12 genes as presented in Table 6.

TABLE 6
List of the 12 genes expressed differentially
in the neuroblastomas of GP and PP patients

| SEQ | | |
|---|---|---|
| ID | | Genbank |
| No. | Sequence description | No. |
| 2 | Ubiquitin-conjugating enzyme E2C | NM_007019 |
| | (UBE2C) | |
| 3 | Insulin-like growth factor binding | NM_001553 |
| | protein 7 (IGFBP7) | |
| 7 | cDNA FLJ30781 fis, clone FEBRA2000874, | AK055343/ |
| | corresponding to the gene encoding | NM_021116 |
| | adenylate cyclase 1 (brain) | |
| 8 | TIF1beta zinc finger protein | X97548 |
| 10 | DKFZp434D1112_s1 434 (synonym: htes3) | AL039831 |
| | cDNA clone DKFZp434D1112 3′ | |
| 20 | Structure specific recognition | NM_003146 |
| | protein 1 (SSRP1) | |
| 22 | Small nuclear ribonucleoprotein | NM_003091 |
| | polypeptides B and B1 (SNRPB) | |
| 25 | Peripheral myelin protein 22 (PMP22), | NM_000304 |
| | transcript variant 1 | |
| 29 | Tripartite motif-containing 2 (TRIM2) | NM_015271 |
| 31 | Polypyrimidine tract binding protein 1 | NM_002819 |
| | (PTBP1), transcript variant 1 | |
| 34 | Erythrocyte membrane protein band | NM_012307 |
| | 4.1-like 3 (EPB41L3) | |
| 37 | Secreted protein, acidic, cysteine- | NM_003118 |
| | rich (osteonectin) (SPARC) | |

The inventors also studied the simultaneous expression of these 12 genes in order to obtain an expression profile. The results are given in FIG. 4. On this dendogram, two groups having two different expression profiles are observed: a first group that makes it possible to classify the patients having a good prognosis (GP) and a second group that makes it possible to classify the patients having a poor prognosis (PP).

A fourth panel comprised 9 genes as presented in Table 7.

TABLE 7
List of the 9 genes expressed differentially
in the neuroblastomas of GP and PP patients

| SEQ | | |
|---|---|---|
| ID | | Genbank |
| No. | Sequence description | No. |
| 2 | Ubiquitin-conjugating enzyme E2C (UBE2C) | NM_007019 |
| 3 | Insulin-like growth factor binding | NM_001553 |
| | protein 7 (IGFBP7) | |
| 7 | cDNA FLJ30781 fis, clone FEBRA2000874, | AK055343/ |
| | corresponding to the gene encoding | NM_021116 |
| | adenylate cyclase 1 (brain) | |
| 8 | TIF1beta zinc finger protein | X97548 |
| 10 | DKFZp434D1112_s1 434 (synonym: htes3) | AL039831 |
| | cDNA clone DKFZp434D1112 3′ | |
| 22 | Small nuclear ribonucleoprotein | NM_003091 |
| | polypeptides B and B1 | |
| 25 | Peripheral myelin protein 22 (PMP22), | NM_000304 |
| | transcript variant 1 | |
| 29 | Tripartite motif-containing 2 (TRIM2) | NM_015271 |
| 34 | Erythrocyte membrane protein band 4.1- | NM_012307 |
| | like 3 (EPB41L3) | |

The inventors also studied the simultaneous expression of these 9 genes in order to obtain an expression profile. The results are given in FIG. 5. On this dendogram, two groups having two different expression profiles are observed: a first group that makes it possible to classify the patients having a good prognosis (GP) and a second group that makes it possible to classify the patients having a poor prognosis (PP).

The discriminatory capacity of all these panels of genes was validated with “test” tumours as described above: all the “PP-test” patients classified according to their expression profile as patients having a poor prognosis proved to be patients having a poor prognosis, and all the “GP-test” patients classified according to their expression profile as patients having a good prognosis proved to be patients having a good prognosis.

These results demonstrate that the prognosis of a neuroblastoma can be determined by analysing the expression of all or some of the 37 genes of sequence SEQ ID Nos. 1 to 37. In particular, the analysis of the expression of the 9 genes of SEQ ID Nos. 2; 3; 7; 8; 10; 22; 25; 29 and 34 makes it possible to discriminate very effectively between patients having a good prognosis and patients having a poor prognosis.

1. Method for neuroblastoma prognosis in a patient suffering from neuroblastoma, characterized in that it comprises the following steps:
a. biological material is extracted from a biological sample taken from the patient,
b. the biological material is brought into contact with at least one specific reagent chosen from the reagents specific for the target genes exhibiting a nucleic acid sequence having any one of SEQ ID Nos. 1 to 37, it being understood that, when the target gene exhibits a nucleic acid sequence having one of SEQ ID Nos. 11, 17 or 37, the biological material is brought into contact with at least two specific reagents chosen from the reagents specific for the target genes exhibiting a nucleic acid sequence having any one of SEQ ID Nos. 1 to 37,
c. the expression of at least one of said target genes is determined, it being understood that, when the target gene exhibits a nucleic acid sequence having one of SEQ ID Nos. 11, 17 or 37, the expression of at least two of said target genes is determined.

2. Method for neuroblastoma prognosis according to claim 1, characterized in that the biological sample taken from the patient is a tissue sample.

3. Method according to claim 1, characterized in that the biological material extracted during step a) comprises nucleic acids.

4. Method according to claim 3, characterized in that the at least one specific reagent of step b) comprises at least one hybridization probe.

5. Method according to claim 4, characterized in that the at least one hybridization probe is immobilized on a support.

6. Method according to claim 5, characterized in that the support is a biochip.

7. Method according to claim 1, characterized in that, during step b), the biological material is brought into contact with at least 37 specific reagents chosen from the reagents specific for the target genes exhibiting a nucleic acid sequence having any one of SEQ ID Nos. 1 to 37, and, during step c, the expression of at least 37 of said target genes is determined.

8. Method according to claim 1, characterized in that, during step b), the biological material is brought into contact with at least 19 specific reagents chosen from the reagents specific for the target genes exhibiting a nucleic acid sequence having SEQ ID No. 1; SEQ ID No. 2; SEQ ID No. 3; SEQ ID No. 7; SEQ ID No. 8; SEQ ID No. 9; SEQ ID No. 10; SEQ ID No. 14; SEQ ID No. 16; SEQ ID No. 20; SEQ ID No. 21; SEQ ID No. 22; SEQ ID No. 25; SEQ ID No. 27; SEQ ID No. 29; SEQ ID No. 31; SEQ ID No. 34; SEQ ID No. 36 or SEQ ID No. 37, and, during step c), the expression of at least 19 of said target genes is determined.

9. Method according to claim 1, characterized in that, during step b), the biological material is brought into contact with at least 9 specific reagents chosen from the reagents specific for the target genes exhibiting a nucleic acid sequence having SEQ ID No. 2; SEQ ID No. 3; SEQ ID No. 7; SEQ ID No. 8; SEQ ID No. 10; SEQ ID No. 22; SEQ ID No. 25; SEQ ID No. 29; SEQ ID No. 34 or SEQ ID No. 37, and, during step c), the expression of at least 9 of said target genes is determined.


SEQUENCE LISTING (67 sequences)

SEQ ID NO: 1 - DNA, 2265 bp, Homo sapiens
agtcctgcgatttcgggtgtagagggagcaggggcctgcggggacctggtgtgggtggagtggggacaagcggtggagaagggtacgccagggtcgctgagagactctgttctccctggagggactggttgccatgagagcagccgtctgaggggacgcagcctgcactacgcgccccaagaggctgtgcgtggcgagcaggtcacgtgacgggagcgcgggctttggaaggcggctgaacgtcaggccacccgccgctaagctgagaagggagagcgagcttaggaccgcctgcccggggcaaccccgaaccaagctttagccgccgaggccgcgtgtcccaaaggccagtcatccctcctctgtgttgccatgggaattcaaggcctggccaaactaattgctgatgtggcccccagtgccatccgggagaatgacatcaagagctactttggccgtaaggtggccattgatgcctctatgagcatttatcagttcctgattgctgttcgccagggtggggatgtgctgcagaatgaggagggtgagaccaccagccacctgatgggcatgttctaccgcaccattcgcatgatggagaacggcatcaagcccgtgtatgtctttgatggcaagccgccacagctcaagtcaggcgagctggccaaacgcagtgagcggcgggctgaggcagagaagcagctgcagcaggctcaggctgctggggccgagcaggaggtggaaaaattcactaagcggctggtgaaggtcactaagcagcacaatgatgagtgcaaacatctgctgagcctcatgggcatcccttatcttgatgcacccagtgaggcagaggccagctgtgctgccctggtgaaggctggcaaagtctatgctgcggctaccgaggacatggactgcctcaccttcggcagccctgtgctaatgcgacacctgactgccagtgaagccaaaaagctgccaatccaggaattccacctgagccggattctgcaggagctgggcctgaaccaggaacagtttgtggatctgtgcatcctgctaggcagtgactactgtgagagtatccggggtattgggcccaagcgggctgtggacctcatccagaagcacaagagcatcgaggagatcgtgcggcgacttgaccccaacaagtaccctgtgccagaaaattggctccacaaggaggctcaccagctcttcttggaacctgaggtgctggacccagagtctgtggagctgaagtggagcgagccaaatgaagaagagctgatcaagttcatgtgtggtgaaaagcagttctctgaggagcgaatccgcagtggggtcaagaggctgagtaagagccgccaaggcagcacccagggccgcctggatgatttcttcaaggtgaccggctcactctcttcagctaagcgcaaggagccagaacccaagggatccactaagaagaaggcaaagactggggcagcagggaagtttaaaaggggaaaataaatgtgtttccccattatacctccttcaccccagaatatttgccgtcttgtacccttaagagctacagctagagaaaccttcacggggtggagagaggattctaaggcttttctagcgtgacccttttcagtagtgctagtcccttttttacttgatcttaatggcaagaaggccacagaggtacttttccttttttagctcaggaaaatatgtcaggctcaaaccacttctcaggcagtttaatggacactaagtccattgttacatgaaagtgatagatagcaacaagttttggagaagagagagggagataaaagggggagacaaaagatgtacagaaatgatttcctggctggccaactggtggccagtgggaggtgatggtggacctagactgtgcttttctgtcttgttcagccttgacccaccttgagagagagccaccaggaaggcgcatcttagcagatgggaggaactgctgagagaagatgggcagaaagctggagcccctggagttggctgtgtctgtgtttgtgactgattactggctgtgtcttgggtgggcagaaactcgaacttgctatgtaatttgtgtctagttattcagaggagtaagatggtgatgttcacctggcaatcagctgagttgagactttggaataagacactggttttcatgcgctgtttttgttttaaagttatgaagaaaaaagtcaataaaattctaaaagtaaaaaaaaaaaaaaaaaa

SEQ ID NO: 2 - DNA, 783 bp, Homo sapiens
ggcacgagcgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatgatgtctggcgataaagggatttctgccttccctgaatcagacaaccttttcaaatgggtagggaccatccatggagcagctggaacagtatatgaagacctgaggtataagctctcgctagagttccccagtggctacccttacaatgcgcccacagtgaagttcctcacgccctgctatcaccccaacgtggacacccagggtaacatatgcctggacatcctgaaggaaaagtggtctgccctgtatgatgtcaggaccattctgctctccatccagagccttctaggagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtccttttttaaaaaaaaaaaaaaaaaaa

SEQ ID NO: 3 - DNA, 1124 bp, Homo sapiens
gccgctgccaccgcaccccgccatggagcggccgtcgctgcgcgccctgctcctcggcgccgctgggctgctgctcctgctcctgcccctctcctcttcctcctcttcggacacctgcggcccctgcgagccggcctcctgcccgcccctgcccccgctgggctgcctgctgggcgagacccgcgacgcgtgcggctgctgccctatgtgcgcccgcggcgagggcgagccgtgcgggggtggcggcgccggcagggggtactgcgcgccgggcatggagtgcgtgaagagccgcaagaggcggaagggtaaagccggggcagcagccggcggtccgggtgtaagcggcgtgtgcgtgtgcaagagccgctacccggtgtgcggcagcgacggcaccacctacccgagcggctgccagctgcgcgccgccagccagagggccgagagccgcggggagaaggccatcacccaggtcagcaagggcacctgcgagcaaggtccttccatagtgacgccccccaaggacatctggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatgaaataccagtgaaaaaaggtgaaggtgccgagctataaacctccagaatattattagtctgcatggttaaaagtagtcatggataactacattacctgttcttgcctaataagtttcttttaatccaatccactaacactttagttatattcactggttttacacagagaaatacaaaataaagatcacacatcaagactatctacaaaaatttattatatatttacagaagaaaagcatgcatatcattaaacaaataaaatactttttatcacaaaaaaaaaaaaaaaa

SEQ ID NO: 4 - DNA, 5084 bp, Homo sapiens
agcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtcccgctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcatggtgctgtaggtgcccctggtcctgctggagccacaggtgaccggggcgaagctggggctgctggtcctgctggtcctgctggtcctcggggaagccctggtgaacgtggcgaggtcggtcctgctggccccaacggatttgctggtccggctggtgctgctggtcaaccgggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctacctggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacggttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccatgaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaagttgtcctcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 5 - DNA, 2518 bp, Homo sapiens
cttcgggtgtacgtgctccgggatcttcagcacccgcggccgccatcgccgtcgcttggcttcttctggactcatctgcgccacttgtccgcttcacactccgccgccatcatggtgaagctcgcgaaggcaggtaaaaatcaaggtgaccccaagaaaatggctcctcctccaaaggaggtagaagaagatagtgaagatgaggaaatgtcagaagatgaagaagatgatagcagtggagaagaggtcgtcatacctcagaagaaaggcaagaaggctgctgcaacctcagcaaagaaggtggtcgtttccccaacaaaaaaggttgcagttgccacaccagccaagaaagcagctgtcactccaggcaaaaaggcagcagcaacacctgccaagaagacagttacaccagccaaagcagttaccacacctggcaagaagggagccacaccaggcaaagcattggtagcaactcctggtaagaagggtgctgccatcccagccaagggggcaaagaatggcaagaatgccaagaaggaagacagtgatgaagaggaggatgatgacagtgaggaggatgaggaggatgacgaggacgaggatgaggatgaagatgaaattgaaccagcagcgatgaaagcagcagctgctgcccctgcctcagaggatgaggacgatgaggatgacgaagatgatgaggatgacgatgacgatgaggaagatgactctgaagaagaagctatggagactacaccagccaaaggaaagaaagctgcaaaagttgttcctgtgaaagccaagaacgtggctgaggatgaagatgaagaagaggatgatgaggacgaggatgacgacgacgacgaagatgatgaagatgatgatgatgaagatgatgaggaggaggaagaagaggaggaggaagagcctgtcaaagaagcacctggaaaacgaaagaaggaaatggccaaacagaaagcagctcctgaagccaagaaacagaaagtggaaggcacagaaccgactacggctttcaatctctttgttggaaacctaaactttaacaaatctgctcctgaattaaaaactggtatcagcgatgtttttgctaaaaatgatcttgctgttgtggatgtcagaattggtatgactaggaaatttggttatgtggattttgaatctgctgaagacctggagaaagcgttggaactcactggtttgaaagtctttggcaatgaaattaaactagagaaaccaaaaggaaaagacagtaagaaagagcgagatgcgagaacacttttggctaaaaatctcccttacaaagtcactcaggatgaattgaaagaagtgtttgaagatgctgcggagatcagattagtcagcaaggatgggaaaagtaaagggattgcttatattgaatttaagacagaagctgatgcagagaaaacctttgaagaaaagcagggaacagagatcgatgggcgatctatttccctgtactatactggagagaaaggtcaaaatcaagactatagaggtggaaagaatagcacttggagtggtgaatcaaaaactctggttttaagcaacctctcctacagtgcaacagaagaaactcttcaggaagtatttgagaaagcaacttttatcaaagtaccccagaaccaaaatggcaaatctaaagggtatgcatttatagagtttgcttcattcgaagacgctaaagaagctttaaattcctgtaataaaagggaaattgagggcagagcaatcaggctggagttgcaaggacccaggggatcacctaatgccagaagccagccatccaaaactctgtttgtcaaaggcctgtctgaggataccactgaagagacattaaaggagtcatttgacggctccgttcgggcaaggatagttactgaccgggaaactgggtcctccaaagggtttggttttgtagacttcaacagtgaggaggatgccaaggaggccatggaagacggtgaaattgatggaaataaagttaccttggactgggccaaacctaagggtgaaggtggcttcgggggtcgtggtggaggcagaggcggctttggaggacgaggtggtggtagaggaggccgaggaggatttggtggcagaggccggggaggctttggagggcgaggaggcttccgaggaggcagaggaggaggaggtgaccacaagccacaaggaaagaagacgaagtttgaatagcttctgtccctctgctttcccttttccatttgaaagaaaggactctggggtttttactgttacctgatcaatgacagagccttctgaggacattccaagacagtatacagtcctgtggtctccttggaaatccgtctagttaacatttcaagggcaataccgtgttggttttgactggatattcatataaactttttaaagagttgagtgatagagctaacccttatctgtaagttttgaatttatattgtttcatcccatgtacaaaaccattttttcctac

SEQ ID NO: 6 - DNA, 3677 bp, Homo sapiens
cgcctgcccgcccgcccgctcgcccccggtccggactcctcctcctcctcttctcgccattgcagttggacccagcagcccggcgcgcaccgcgtggcttttgggggcagaccccggcgggctgtggcaggagggcggcggcggcggctgcggtcgaagaaggggacgccgacaagagttgaagtattgataacaccaaggaactctatcacaatttgaaaagataagcaaaagtttgatttccagacactacagaagaagtaaaaatgcgtccaatgcgaatttttgtgaatgatgaccgccatgtgatggcaaagcattcttccgtttatccaacacaagaggagctggaggcagtccagaacatggtgtcccacacggagcgggcgctcaaagctgtgtccgactggatagacgagcaggaaaagggtagcagcgagcaggcagagtccgataacatggatgtgcccccagaggacgacagtaaagaaggggctggggaacagaagacggagcacatgaccagaaccctgcggggagtgatgcgggtgggcctggtggcaaagggcctcctactcaagggggacttggatctggagctggtgctgctgtgtaaggagaagcccacaaccgccctcctggacaaggtggccgacaacctggccatccagcttgctgctgtaacagaagacaagtacgaaatactgcaatctgtcgacgatgctgcgattgtgataaaaaacacaaaagagcctccattgtccctgaccatccacctgacatcccctgttgtcagagaagaaatggagaaagtattagctggagaaacgctatcagtcaacgaccccccggacgttctggacaggcagaaatgccttgctgccttggcgtccctccgacacgccaagtggttccaggccagagccaacgggctgaagtcttgtgtcattgtgatccgggtcttgagggacctgtgcactcgcgtgcccacctggggtcccctccgaggctggcctctcgagctcctgtgtgagaaatccattggcacggccaacagaccgatgggtgctggcgaggccctgcggagagtgctggagtgcctggcgtcgggcatcgtgatgccagatggttctggcatttatgacccttgtgaaaaagaagccactgatgctattgggcatctagacagacagcaacgggaagatatcacacagagtgcgcagcacgcactgcggctcgctgccttcggccagctccataaagtcctaggcatggaccctctgccttccaagatgcccaagaaaccaaagaatgaaaacccagtggactacaccgttcagatcccaccaagcaccacctatgccattacgcccatgaaacgcccaatggaggaggacggggaggagaagtcgcccagcaaaaagaagaagaagattcagaagaaagaggagaaggcagagcccccccaggctatgaatgccctgatgcggttgaaccagctgaagccagggctgcagtacaagctggtgtcccagactgggcccgtccatgcccccatctttaccatgtctgtggaggttgatggcaattcattcgaggcctctgggccctccaaaaagacggccaagctgcacgtggccgttaaggtgttacaggacatgggcttgccgacgggtgctgaaggcagggactcgagcaagggggaggactcggctgaggagaccgaggcgaagccagcagtggtggcccctgccccagtggtagaagctgtctccacccctagtgcggcctttccctcagatgccactgccgagcaggggccgatcctgacaaagcacggcaagaacccagtcatggagctgaacgagaagaggcgtgggctcaagtacgagctcatctccgagaccgggggcagccacgacaagcgcttcgtcatggaggtcgaagtggatggacagaagttccaaggtgctggttccaacaaaaaggtggcgaaggcctacgctgctcttgctgccctagaaaagcttttccctgacacccctctcgcccttgatgccaacaaaaagaagagagccccagtacccgtcagagggggaccgaaatttgctgctaagccacataaccctggcttcggcatgggaggccccatgcacaacgaagtgcccccaccccccaaccttcgagggcggggaagaggcgggagcatccggggacgagggcgcgggcgaggatttggtggcgccaaccatggaggctacatgaatgccggtgctgggtatggaagctatgggtacggaggcaactcggcgacagcaggctacagtgactttttcacagactgctacggctatcatgattttgggtcttcctagagcgtctaaaagtattgcacacaaaatcaactttttactccaatttcctccaactccaaaacccaaagtgtccgtgctgtgtccctgtgcttcactgggtttctcaaccgtggcttttcaccgcagcttgtctgaaactcttagcctgcagaatttaagacaatggcagtttttatcgtgatttgcctttgaacttggtcctattgaagttcacaataagtggaaaacaattttttcagagaatgtatttttgtgcagaattgcacagaattctagagacagcgttgttcggcatcaaggcaaaagcccacctttgctttttatggaaagcattactttatttaaagagacagacaatgacgcattttaatctacctttgtcttaatttacagcaggttttgtatgaatttttaaccttttaacaaactcccaaatctggttgatgcctttgacagtgatgaaaacgatttcaccacatctgaatccagagaaaccggctttttttcttattgcgagcatgttaaaacgttgggaacatgtggggaattgtatattgcgctgaattaacttctcccgcctcttgtaatgctctggtgggttcttgtttgggaatgcgatattttgtggctggtttagctagagagtgaactctcaaaggtatcaaaactgtgcttccattattagtgcaagaaacagacaggctttaaggggtagatgacgtgaaattttgcaagtcttaattacagctgcagatgcatgggattctggatttttttgttgctttttagtttaatgggactttaaaagtaattgaggagaaagaaccgtgatgttccctgtttctccagtaaaggactggcttttgcttgggcagaggtggtgctgctgggtgtgcagctgccacagactccaaaggcgtagaagtttgtgccaacacacggagtcattctggctctctgctgaggcccctgttttctggcaggtgccctccttggaaactggttttggctctgatcagcggttctttttgcagcaaagcctgcatctgtgttgacttgcaagattttgcgtttattcaggcaaaaactggtcaaaatggttactacatgatttgttcccagaggtttgaaacattcagtgaaactttttaaaactttgattgcatgatgtattttttttttagaaagttattgtttgagaataatgtctttttataccaggaaaatagttatcctgaatgacgttgaaaactccccctcccctttatttttttttaatcaatacatgtgaaagtaacaagc

SEQ ID NO: 7 - DNA, 2901 bp, Homo sapiens
ttgaaatcaggaaatcaggccgggcgcagtggctcatgcctgtaagcccagcactttgggaggcggaggcgggtggatccattgaggtcaggagctcaagaccagcctggtcaacatggtgaaaccccgtctctactaaaaatacaaaaaaaaaattagctgggcgtgttggcgggagcctgtagtcccagctacacgggaggctaaggtgggagaattgcttgaacccgggaggcggaggttgcagtgagctgagattgcaccattgcactccagcctgggcgacagagcaagactctctcaaaaaaaaaaaaaaagaaagaaagaaatcagaaaatcgaccacagtggtagccacctggcctaatgctgtgtttttgtacctgacaggggtcactcattttaggcacaactccttcattctttgtgaaattagtgagtttccttctacccgtcaccagattcaatatgttctattaatacaccgataaccacaggggaagggcacttgtcgctctcccacctggttaccacagtctccatgggtcttttgccgtgaccacaaataaaggaaacactcatcactagtatctaagtcgggctttacagtaactatgcaccttctgtgtgcttcacctcactctctacttcaaacagcccatggagggaggtattattatactccttatgttgacagtgaagaatctgaggcccagagaggttggggacttgagtaaagtcacacagccctgagaggcaggaccagggttccattcctgctctatccagttccaagcccttgtgttttccattatgtttagtgcctctttgctaacagcaacatctgcaagatttgtgttggttttgatggagaactctagctcatccacatgctagtgcccaagtggtggaggggccacctcagcaggtgggttctgaatgcagccaaggctgtccccgcaatgggtgagactcgctccaactgcccgccctcagagcaggtgcctaagtcctccctggcactggcaggccttacctcacattgctaaattaaagcaatgcaattcctcttgggtaagaggaattcctccttctttactaactgatccccagcaaggaaataaaatgttaggctttaaaaatccctactttgtcatatcagactatattctaaaactatatttgagcgaaacctgtcattgcgtctaatttcaaatatacagaatctccttaagagctgttgccttatttttttgtaaagcctctctgacatcaaatggggagaaatggtggcacctccagacaccctgaaactacacaccatttcttccctgctcagcttctgctcaggagttctgtgagctatgggaaggccattggttgtatttgctacttttactttcatcttcctctgctgtagagccatttaatgttattgtcatatgctgctggtgaggtaaaggtgggtccgggtgccttcccaggggttagaggatgttcaaagggccgatttcagcaggagttcagagggcttatgatggatggtgagagatttgacaaccaccagagcacatgtgctctgaccctctcctgggcattggttcctgctggtaccgggcggttcagaccttcaaataggttgctttcaaaagagctttcaggcacttattgagaattaatgtttaaacagacataatagcctagatgaactcccaagagatctattaaatcttgtgggctgaataaatatctcgtgcaggactgtgcaacagtagcccagagcatcctgcctgtgggcatccacctcccaggtgagggcagtgggaagctggcccgacggcagccagaacttgtttctcacctcccaccagcaaccccccacccaactctgggccccaggcacacgaagcacaagtctcaggggaccattcccacattgggggatcctgagggagcccatcaccgcctcttgcatacaactgtccactaggaggcacgcccagtgtgggagagatgtatggtcttgccttccacctgtaaaaactgcacatatgcaagccatttgcactctggaactgcatgccgtgaaaactcctaatggtgtggaacttagtttgaatttgaaatcacgccgcatgcacaaagggacaggcccaggcccgacctcaggtcatccgcccgctggctgcagagcatccctgggagccaaggcgaggcccgtggagcctgagctttgtgtagctcgagctttgtgtagctcgtgcacttattatgcaccacctcccttcagtcaccactcctcttcctccgccatcctcatttatactgattgcacaccccccgctcaaacaacaatgtccttattatgatgaccatctcgtagtggtacattccattcctatttaaggtaagcccaaagcccacttttggattttctcgactgtccgagaaaagttgtgtaagcgcctgcgttcttctgggtttggctagatagggttgtgtccctctatggaatggagagtgatgtgggcaagggtgtcattttctcgcacaatacaactcactgaggatgcttctgtagaagtgagaaacacgatgagtacattcagaattacaataactcactctcactgggtaacttctcatgatagatttgtatgatcaatacgggtctatttttatgtcaactgaacactgtagggtaccttccagtctttttcaagattgttaaattgagacaagtaattgaataatttgtcctatttttattttaaaaaaagtgaatggactgaaatgttaaatgtgaatgtacatttcttaattgcaatttttctactgagtgtttgcactatactttctggaatcttatttaacaaaaataaagggaaaaaattgcttgact

SEQ ID NO: 8 - DNA, 3056 bp, Homo sapiens
gcggggcgggccggcggcggaggccgggccgcggagccaggagtgactagcagcagttggccgtgccgtagcagcgtcccgcgcgcggcgggcagcggcccaggaggcgcgtggtgcgggtttcggcggcggctgaggaagaagcgcgggcggcgccttcgggaggcgagcaggcagcagttggccgtgccgtagcagcgtcccgcgcgcggcgggcagcggcccaggaggcgcgtggcggcgctcggcctcgcggcggcggcggcggcagcggcccagcagttggcggcgagcgcgtctgcgcctgcgcggcgggccccgcgcccctcctccccccctgggcgcccccggcggcgtgtgaatggcggcctccgcggcggcagcctcggcagcagcggcctcggccgcctctggcagcccgggcccgggcgagggctccgctggcggcgaaaagcgctccaccgccccttcggccgcagcctcggcctctgcctcagccgcggcgtcgtcgcccgcggggggcggcgccgaggcgctggagctgctggagcactgcggcgtgtgcagagagcgcctgcgacccgagagggagccccgcctgctgccctgtttgcactcggcctgtagtgcctgcttagggcccgcggcccccgccgccgccaacagctcgggggacggcggggcggcgggcgacggcaccgtggtggactgtcccgtgtgcaagcaacagtgcttctccaaagacatcgtggagaattatttcatgcgtgatagtggcagcaaggctgccaccgacgcccaggatgcgaaccagtgctgcactagctgtgaggataatgccccagccaccagctactgtgtggagtgctcggagcctctgtgtgagacctgtgtagaggcgcaccagcgggtgaagtacaccaaggaccatactgtgcgctctactgggccagccaagtctcgggatggtgaacgtactgtctattgcaacgtacacaagcatgaaccccttgtgctgttttgtgagagctgtgatactctcacctgccgagactgccagctcaatgcccacaaggaccaccagtaccagttcttagaggatgcagtgaggaaccagcgcaagctcctggcctcactggtgaagcgccttggggacaaacatgcaacattgcagaagagcaccaaggaggttcgcagctcaatccgccaggtgtctgacgtacagaagcgtgtgcaagtggatgtcaagatggccatcctgcagatcatgaaggagctgaataagcggggccgtgtgctggtcaatgatgcccagaaggtgactgaggggcagcaggagcgcctggagcggcagcactggaccatgaccaagatccagaagcaccaggagcacattctgcgctttgcctcttgggctctggagagtgacaacaacacagcccttttgctttctaagaagttgatctacttccagctgcaccgggccctcaagatgattgtggatcccgtggagccacatggcgagatgaagtttcagtgggacctcaatgcctggaccaagagtgccgaggcctttggcaagattgtggcagagcgtcctggcactaactcaacaggccctgcacccatggcccctccaagagccccagggcccctgagcaagcagggctctggcagcagccagcccatggaggtgcaggaaggctatggctttgggtcaggagatgatccctactcaagtgcagagccccatgtgtcaggtgtgaaacggtcccgctcaggtgagggcgaggtgagcggccttatgcgcaaggtgccacgagtgagccttgaacgcctggacctggacctcacagctgacagccagccacccgtcttcaaggtcttcccaggcagtaccactgaggactacaaccttattgttattgaacgtggcgctgccgctgcagctaccggccagccagggactgcgcctgcaggaacccctggtgccccacccctggctggcatggccattgtcaaggaggaggagacggaggctgccattggagcccctcctactgccactgagggccctgagaccaaacctgtgcttatggctcttgcggagggtcctggtgctgagggtccccgcctggcctcacctagtggcagcaccagctcagggctggaggtggtggctcctgagggtacctcagccccaggtggtggcccgggaaccctggatgacagtgccaccatttgccgtgtctgccagaagccaggcgatctggttatgtgcaaccagtgtgagttttgtttccacctggactgtcacctgccggccctgcaggatgtaccaggggaggagtggagctgctcactctgccatgtgctccctgacctgaaggaggaggatggcagcctcagcctggatggtgcagacagcactggcgtggtggccaagctctcaccagccaaccagcggaaatgtgagcgtgtactgctggccctattctgtcacgaaccctgccgccccctgcatcagctggctaccgactccaccttctccctggaccagcccggtggcaccctggatctgaccctgatccgtgcccgcctccaggagaagttgtcacctccctacagctccccacaggagtttgcccaggatgtgggccgcatgttcaagcaattcaacaagttaactgaggacaaggcagacgtgcagtccatcatcggcctgcagcgcttcttcgagacgcgcatgaacgaggccttcggtgacaccaagttctctgctgtgctggtggagcccccgccgatgagcctgcctggtgctggcctgagttcccaggagctgtctggtggccctggtgatggcccctgaggctggagcccccatggccagcccagcctggctctgttctctgtcctgtcaccccatccccactcccctggtggcctgactcccactccctggtggccccatcccccagttcctcacgatatggtttttacttctgtggatttaataaaaaaaacttcaccagttcaaaaaaaaaaaaaaaaaaaaa

SEQ ID NO: 9 - DNA, 3149 bp, Homo sapiens
agcggaatctcggaaaggcgagaaagaagctgtctccatcttgtctgtatccgctgctcttgtgacgttgtggagatggggagcgtcctggggctgtgctccatggcgagctggataccatgtttgtgtggaagtgccccgtgtttgctatgccgatgctgtcctagtggaaacaactccactgtaactagattgatctatgcacttttcttgcttgttggagtatgtgtagcttgtgtaatgttgataccaggaatggaagaacaactgaataagattcctggattttgtgagaatgagaaaggtgttgtcccttgtaacattttggttggctataaagctgtatatcgtttgtgctttggtttggctatgttctatcttcttctctctttactaatgatcaaagtgaagagtagcagtgatcctagagctgcagtgcacaatggattttggttctttaaatttgctgcagcaattgcaattattattggggcattcttcattccagaaggaacttttacaactgtgtggttttatgtaggcatggcaggtgccttttgtttcatcctcatacaactagtcttacttattgattttgcacattcatggaatgaatcgtgggttgaaaaaatggaagaagggaactcgagatgttggtatgcagccttgttatcagctacagctctgaattatctgctgtctttagttgctatcgtcctgttctttgtctactacactcatccagccagttgttcagaaaacaaggcgttcatcagtgtcaacatgctcctctgcgttggtgcttctgtaatgtctatactgccaaaaatccaagaatcacaaccaagatctggtttgttacagtcttcagtaattacagtctacacaatgtatttgacatggtcagctatgaccaatgaaccagaaacaaattgcaacccaagtctactaagcataattggctacaatacaacaagcactgtcccaaaggaagggcagtcagtccagtggtggcatgctcaaggaattataggactaattctctttttgttgtgtgtattttattccagcatccgtacttcaaacaatagtcaggttaataaactgactctaacaagtgatgaatctacattaatagaagatggtggagctagaagtgatggatcactggaggatggggacgatgttcaccgagctgtagataatgaaagggatggtgtcacttacagttattccttctttcacttcatgcttttcctggcttcactttatatcatgatgacccttaccaactggtacaggtatgaaccctctcgtgagatgaaaagtcagtggacagctgtctgggtgaaaatctcttccagttggattggcatcgtgctgtatgtttggacactcgtggcaccacttgttcttacaaatcgtgattttgactgagtgagacttctagcatgaaagtcccactttgattattgcttatttgaaaacagtattcccaacttttgtaaagttgtgtatgtttttgcttcccatgtaacttctccagtgttctggcatgaattagattttactgcttgtcattttgttattttcttaccaagtgcattgatatgtgaagtagaatgaattgcagaggaaagttttatgaatatggtgatgagttagtaaaagtggccactattgggcttattctctgctctatagttgtgaaatgaagagtgaaaacaaatttgtttgactattttaaaattatattagaccttaagctgttttagcaagcattaaagcaaatgtatggctgccttttaaaatatttgatgtgttgcctggcaggatactgcaaagaacatggtttattttaaaatttataaacaagtcacttaaatgccagttgtctgaaaaatcttataaggttttacccttgatacggaatttacacaggtagggagtgtttagtggacaatagtgtaggttatggatggaggtgtcggtactaaattgaataacgagtaaataatcttacttgggtagagatggcctttgccaacaaagtgaactgttttggttgttttaaactcatgaagtatgggttcagtggaaatgtttggaactctgaaggatttagacaaggttttgaaaaggataatcatgggttagaaggaagtgtttgaaagtcactttgaaagttagttttgggccagcacggtagctcacccttgtaatcccagcactttgggaggctgaggtgggtagattacttgagcccaggaattcaagaccagcctgggcaacatggtgaaaccctgtttctataaaaaataatctgggctttgtagcatatgcctgtggtcccagctactgaggaggctgaggtgggaggattgcttgagcccaggaggcagaggttgcagtgagccaaggtcacgtcactgcactctagcctgggcaacagagtaagacaaaaaaatatatatatattgaaaatcaaaggaggcaaaattttgacagggaaggaagtaactgcaaaacactaggctttagtaggtacttatataaaatctagtccagttctctcatttaaaaaaatgaagacactgaagtacagacttaaatagctcagatagctaattaggaaatttcaagttggccaataatagcattctctctgacatttaaaaataatttctattcaaaatacatgcataattgattttacacctcattactggtggataatttatgtgatgtggattgctggtgtccagcatgacccataaacaggtcagaagaatgatggaatgttttagaataaactcctgcttatagtatactacacagttcaaaagatgtttaaaatgcttttgtatttactgccatgtaattgaaatatatagattattgtaacctttcaacctgaaaatcaagcagtatgagagtttagttatttgtatgtgtcactagtgtctaatgaagcttttaaaatctacaatttcttctttaaaaatatttattaatgtgaatggaatataacaattcagcttaattccccaaccttattctgtgtgtagacattgtattccacaattttgaatggctgtgttttacctctaaataaatgaattcagagaaagtgaaaaaaaaaaaaaaaa

SEQ ID NO: 10 - DNA, 580 bp, Homo sapiens
cttttttttttttttttttaaagtctttagtatatttatttgtataaagagtaaacaaagtgcatatagagtggccacaggtttgacacagagaccttggtgatgtaggctatgaacaaatttaaatggcaacttcattgctgccactgaaccaatcctgaatttgggctcaacaggtgaaaagtaacaatatcaaacgaatactaaacagcataacaaaaagattttcagactcttggtcataaagaccgtaatcgttcacattgaatcaatgactaaacatttttgattacccagctacctccaagcaaactgaaaactgtctagtggatcctgaagtccatagtgcctctagccgggtctttcaagtgttgcaccacagggtgatgattgatggtaaaaacagggatcaacccttgtagatcggtggtaagtatggaaaccctctaagaacagtgcagcgtatgtggtattcagactggttgcatacagcattcaaaaccagtgctggaatagcttgccccaaagtggtagagttataaaaggatatacattgacgtttcttaaaagcatgtgtaat

SEQ ID NO: 11 - DNA, 2467 bp, Homo sapiens
ggcacgaggctccggtgtgtctgtcggttgcagtgttggaggtcggcgccggcccccgccttccgcgccccccacgggaaggaagcacccccggtattaaaacgaacggggcggaaagaagccctcagtcgccggccgggaggcgagccgatgccgagctgctccacgtccaccatgccgggcatgatctgcaagaacccagacctcgagtttgactcgctacagccctgcttctacccggacgaagatgacttctacttcggcggccccgactcgacccccccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgctagacgcttctcaaaactggacagtcactgccactttgcacattttgattttttttttaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttctttttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggaagttcatacctaagtactgtaataatacctcaatgtttgaggagcatgttttgtatacaaatatattgttaatctctgttatgtactgtactaattcttacactgcctgtatactttagtatgacgctgatacataactaaatttgatacttatattttcgtatgaaaatgagttgtgaaagttttgagtagatattactttatcactttttgaactaagaaacttttgtaaagaaatttactatatatatatgcctttttcctagcctgtttcttcctgttaatgtatttgttcatgtttggtgcatagaactgggtaaatgcaaagttctgtgtttaatttcttcaaaatgtatatatttagtgctgcatcttatagcactttgaaatacctcatgtttatgaaaataaatagcttaaaattaaaaaaaaaaaaaaaaaa

SEQ ID NO: 12 - DNA, 762 bp, Homo sapiens
taccattcttcaagaaacggtttgaatcagactgcctttccttttgtcttcattgtcataaacatctgcccccgtgtggttctgactggccgcgaacccctacccgaagcttttattccatcattgtgcaccgttggtggggaatgctgtggcaacaggccacgcctccacttactggttggctttgcgcaggcgccaacggaagtgggtcgcaggaagaggaagtcccgcctctctctcctcaggcagcagcaacgcggaggaaacgggagtgaacggagagcgtagtgaccatcatgagcctcctcaacaagcccaagagtgagatgaccccagaggagctgcagaagcgagaggaggaggaatttaacaccggtccactctctgtgctcacacagtcagtcaagaacaatacccaagtgctcatcaactgccgcaacaataagaaactcctgggccgcgtgaaggccttcgataggcactgcaacatggtgctggagaacgtgaaggagatgtggactgaggtacccaagagtggcaagggcaagaagaagtccaagccagtcaacaaagaccgctacatctccaagatgttcctgcgcggggactcagtcatcgtggtcctgcggaacccgctcatcgccggcaagtaggggccgcctgtctgttgacagaactcactcctctgtcctatgaagaccgctgccattggtgttgagaataataaagctctgtgtttttttctaaaaaaaaaaaaaaaaa

SEQ ID NO: 13 - DNA, 3379 bp, Homo sapiens
aattccgcggaatcatcggaatccttcaccatggcatccagcccggcccagcgtcggcgaggcaatgatcctctcacctccagccctggccgaagctcccggcgtactgatgccctcacctccagccctggccgtgaccttccaccatttgaggatgagtccgaggggctcctaggcacagaggggcccctggaggaagaagaggatggagaggagctcattggagatggcatggaaagggactaccgcgccatcccagagctggacgcctatgaggccgagggactggctctggatgatgaggacgtagaggagctgacggccagtcgaagggaggcagcagacgggccatgcggcacggtgaccgggagctggccggggctgggcgcatgcgccgtgggctcctgtatgacagcgatgaggaggacgaggagcgccctgcccgcaagcgccgccagtggagccggcacggaggacggcgaggaggacgagcagatgattgagagcatcgagaacctggaggatctcaaaggccactctgtgcgcgagtgggtgagcatggcgggcccccggctggagatccaccaccgcttcaagaacttcctgcgcactcacgtcgacagccacggccacaacgtcttcaaggagcgcatcagcgacatgtgcaaagagaaccgtgagagcctggtggtgaactatgaggacttggcagccagggagcacgtgctggcctacttcctgcctgaggcaccggcggagctgctgcagatctttgatgaggctgccctggaggtggtactggccatgtaccccaagtacgaccgcatcaccaaccacatccatgtccgcatctcccacctgcctctggtggaggagctgcgctcgctgaggcagctgcatctgaaccagctgatccgcaccagtggggtggtgaccagctgcactggcgtcctgccccagctcagcatggtcaagtacaactgcaacaagtgcaatttcgtcctgggtcctttctgccagtcccagaaccaggaggtgaaaccaggctcctgtcctgagtgccagtcggccggcccctttgaggtcaacatggaggagaccatctatcagaactaccagcgtatccgaatccaggagagtccaggcaaagtggcggctcggcggctgccccgctccaaggacgccattctcctcgcagatctggtggacagctgcaacgcaggagacgagatagagctgactggcatctatcacaacaactatgatggctccctcaacactgccaatggcttccctgtctttgccactgtcatcctagccaaccacgtggccaagaaggacaacaaggttgctgtaggggaactgaccgatgaagatgtgaagatgatcactagcctctccaaggatcagcagatcggagagaagatctttgccagcattgctccttccatctatggtcatgaagacatcaagagaggccctgctctggccctgttcggaggggagcccaaaaacccaggtggcaagcacaaggtacgtggtgatatcaacgtgctcttgtgcggagaccctggcacagcgaagtcgcagtttctcaagtatattgagaaagtgtccagccgagccatcttcaccactggccagggggcgtcggctgtggccgtcacggcgtatgtccagcggcaccctgtcagcagggagtggaccttggaggctggggccctggttctggctgaccgaggagtgtgtctcattgatgaatttgacaagatgaatgaccaggacagaaccagcatccatgaggccatggagcaacagagcatctccatctcgaaggctggcatcgtcacctccctgcaggctcgctgcacggtcattgctgccgccaaccccataggagggcgctacgacccctcgctgactttctctgagaacgtggacctcacagagcccatcatctcacgctttgacatcctgtgtgtggtgagggacaccgtggacccagtccaggacgagatgctggcccgcttcgtggtgggcagccacgtcagacaccaccccagcaacaaggaggaggaggggctggccaatggcagcgctgctgagcccgccatgcccaacacgtatggcgtggagcccctgccccaggaggtcctgaagaagtacatcatctacgccaaggagagggtccacccgaagctcaaccagatggaccaggacaaggtggccaagatgtacagtgacctgaggaaagaatctatggcgacaggcagcatccccattacggtgcggcacatcgagtccatgagtcatggcggaggcccacgcgcgcatccatctgcgggactatgtgatcgaagacgacgtcaacatggccatccgcgtgatgctggagagcttcatagacacacagaagttcagcgtcatcgcagcatgcgcaagacttttgcccgctacctttcattccggcgtgacaacaatgagctgttgctcttcatactgaagcagttagtggcagagcaggtgacatatcagcgcaaccgctttggggcccagcaggacactattgaggtccctgagaaggacttggtggataaggctcgtcagatcaacatccacaacctctctgcattttatgacagtgagctcttcaggatgaacaagttcagccacgacctgaaaaggaaaatgatcctgcagcagttctgaggccctatgccatccataaggattccttgggattctggtttggggtggtcagtgccctctgtgctttatggacacaaaaccagagcacttgatgaactcggggtactagggtcagggcttatagcaggatgtctggctgcacctggcatgactgtttgtttctccaagcctgctttgtgcttctcacctttgggtgggatgccttgccagtgtgtcttacttggttgctgaacatcttgccacctccgagtgctttgtctccactcagtaccttggatcagagctgctgagttcaggatgcctgcgtgtggtttaggtgttagccttcttacatggatgtcaggagagctgctgccctcttggcgtgagttgcgtattcaggctgcttttgctcgctttggccagagagctggttgaagatgtttgtaatcgttttcagtctcctgcaggtttctgtgcccctgtggtggaagaggcacgacagtgccagcgcagcgttctgggctcctcagtcgcaggggtgggatgtgagtcatgcggattatccactcgccacagttatcagctgccattgctccctgtctgtttccccactctcttatttgtgcattcggtttggtttctgtagttttaatttttaataaagttgaataaaatataaaaaaaaaaa

SEQ ID NO: 14 - DNA, 1488 bp, Homo sapiens
gttggtgagcatcatggcaaccgttacagccacaaccaaagtcccggagatccgtgatgtaacaaggattgagcgaatcggtgcccactcccacatccggggactggggctggacgatgccttggagcctcggcaggcttcgcaaggcatggtgggtcagctggcggcacggcgggcggctggcgtggtgctggagatgatccgggaagggaagattgccggtcgggcagtccttattgctggccagccgggcacggggaagacggccatcgccatgggcatggcgcaggccctgggccctgacacgccattcacagccatcgccggcagtgaaatcttctccctggagatgagcaagaccgaggcgctgacgcaggccttccggcggtccatcggcgttcgcatcaaggaggagacggagatcatcgaaggggaggtggtggagatccagattgatcgaccagcaacagggacgggctccaaggtgggcaaactgaccctcaagaccacagagatggagaccatctacgacctgggcaccaagatgattgagtccctgaccaaggacaaggtccaggccggggacgtgatcaccatcgacaaggcgacgggcaagatctccaagctgggccgctccttcacacgcgcccgcgactacgacgctatgggctcccagaccaagttcgtgcagtgcccagatggggagctccagaaacgcaaggaggtggtgcacaccgtgtccctgcacgagatcgacgtcatcaactctcgcacccagggcttcctggcgctcttctcaggtgacacaggggagatcaagtcagaagtccgtgagcagatcaatgccaaggtggctgagtggcgcgaggagggcaaggcggagatcatccctggagtgctgttcatcgacgaggtccacatgctggacatcgagagcttctccttcctcaaccgggccctggagagtgacatggcgcctgtcctgatcatggccaccaaccgtggcatcacgcgaatccggggcaccagctaccagagccctcacggcatccccatagacctgctggaccggctgcttatcgtctccaccaccccctacagcgagaaagacacgaagcagatcctccgcatccggtgcgaggaagaagatgtggagatgagtgaggacgcctacacggtgctgacccgcatcgggctggagacgtcactgcgctacgccatccagctcatcacagctgccagcttggtgtgccggaaacgcaagggtacagaagtgcaggtggatgacatcaagcgggtctactcactcttcctggacgagtcccgctccacgcagtacatgaaggagtaccaggacgccttcctcttcaacgaactcaaaggcgagaccatggacacctcctgagttggatgtcatcccccgaccccaccctgttttccaccagagttctgacactgtgactctgtataaaatggttgggaagctgc

SEQ ID NO: 15 - DNA, 1811 bp, Homo sapiens
ggtttgtgtagagaggcgtgcagagcccgttgtccggagtgcacctgctgcctgttctgtccctcccgggagcccccgccgctgtcgccgtcgagtcgccatggaagtgcagaaagaggcacagcgcatcatgaccctgtcggtgtggaagatgtatcactcccgcatgcagcgcggtggcctgcggctgcaccggagtctgcagctgtcgctggtcatgcgcagcgcccgggagctctacctctcggccaaggtggaggccctcgagcccgaggtgtcgttgccggccgccctcccctctgaccctcgcctgcacccgccccgagaagccgagtccacggccgagacagcgacccccgacggtgagcacccgtttccggagccaatggacacgcaggaggcgccgacagccgaggagacctccgcctgctgtgccccgcgccccgccaaagtcagccgcaaacgacgcagcagcagcctgagcgacggcggggacgttggactggtcccgagcaagaaagcccgtctggaagaaaaggaagaagaggagggagcgtcatccgaagtcgccgatcgcctgcagccccctccgggccaagcggagggcgcctttcccaacctggcccgcgtcctgcagaggcgcttctccggcctcctgaactgcagccccgcggcccctccgacggcgccgcccgcgtgcgaggcaaagcccgcttgccgcccggcggacagcatgctcaacgtgctcgtgcgggccgtggtggccttctgaggaccccgagcggcgctgccggagcccagagcgcgcgtcgaaccgtcggcccgagggcgcagacctgaggcgaggccacccccctccatcctgggggaagcgcccgcgaaaaccgtggagagaagccgccgcccgggctgctgagaggcccggagaggactctgtccccggggagccatcgccttcagtgtgcagggacggcaccgaggagtctgagccgggcgcgggcgccttccgcagagacctgcgcccacaggtgctgtcttagtggactgggacgtgaacctttcgctctccttctggactgggagaagggaggcttgggtgttgtgttttttgttttgtttgtttgtttgtttttaaagatctcctcagggtcggacttcattttgtactgtgggctgtgctggccctttcaaggtttttcaagagttggttttgcgtttccaacctcggagaattccaggcactccccttccccctccgctgacatacttgtataagcggtcatcgttgcgtcatggggcaggcgtggggagcttcctgtcgccttggctgggtgtgggcctggaggaaggtcctggggcgtgcactcgcctgggcagtggggaggagagtggcctgagttacttcacccccgcgtgctgctggttaatgtcccgcgtctctgcaccttcgggtgggagcggggactgatctactttcacattctcaagtttttctcatctgcattagaggtccccagtaggttcccaggttccagcgtgcccctccctcagacacacggacacaatcagccgagaagttcctggtctgaatcacgagaatgtggaggggtggggggtgtcagtggaaaggcataaggctgagctgagaccagttgctggtgaaactgggccaatctggggaggggaacatccttgccagggagtttctgagggtctgctttgtttacctttcgtgcggtggattctttttaactccgtctacctggcgttttgttagaaatgtcagataggaaaataaaaaccatttgagt

SEQ ID NO: 16 - DNA, 2038 bp, Homo sapiens
ggcccgcgggactcagaccagcggggagcgcggcctccgcccttggggccctcccgccgggccggagacccaagcccccaacgccaggccctgccctggaagcgctcgcggcccggcgcctggacgggggagttgctgctctttggcgtaaattgcaatcgattagggatcgtttctcagaatcaagttagaagtgagagttcagataagtgaggccgccattgctgctttgaacacctcagaaggggagaatggatttatcaggagtgaaaaagaagagcttgctaggagtcaaagaaaataataaaaagtccagcactagggctccttcacctaccaaacgcaaagaccgctcagatgagaagtccaaggatcgctcaaaagataaaggggccaccaaggagtcgagtgagaaggatcgcggccgggacaaaacccgaaagaggcgcagcgcttccagtggtagcagcagtaccaggtctcggtccagctcgacttccagctcaggctccagcaccagcactggctcaagcagtggctccagctcttcctcagcatccagccgctcaggaagctccagcacctcccgcagctccagctctagcagctcttctggctctccaagtccttctcggcgcagacacgacaacaggaggcgctcccgctccaaatccaaaccacctaaaagagatgaaaaggagaggaaaaggcggagcccatctcctaagcccaccaaagtgcacattgggagactcacccggaatgtgacaaaggatcacatcatggagatattttccacctatgggaaaattaaaatgattgacatgcccgtggaaaggatgcatccccatctgtccaaaggctatgcgtacgtagagtttgagaatccagatgaagccgagaaggcgctgaagcacatggatggaggacaaattgatggccaggagatcactgccaccgccgtgctggccccctggcctaggccaccccccaggagattcagccctcccaggagaatgttgccaccaccgcctatgtggcgcaggtctcccccacggatgaggagaaggtcccgctccccgaggcgcaggtcccccgtgcgccggagatcacggtccccgggccgccgccgccacaggagccgctccagctccaactcctcccgataaacaggccactgaagctctcgcccctgtaacttataccccacccagctcagttttgtcacttttctagccaaaggaagaccagtaggaaagcaaacccttgactctggcaggatttgcaggcagcaggcagcacccctctgccagccgggccccggctgcagaagtgctgttggtttggatgctgtgtgcctgtcaagattccctccggttttctggctagaaagctcatccgtttccggtttctaagagtcagttcagtggcagagccaccagggaaaagtgaggctcttgggggtggtttgaccctgcttacctgggagcacacttttcccttccccgatgacctgggatggtggccaggccgtgcccttgctgttgctgggcagtgtccttttggaaagggagctgccccaggctttagtgcagctgccaaccctgttaggcctggcctctcgaggcctcttctgacctcaagggtcacaccccctcaaagatcctctcacccatggtagttgctgctcgtggttctgtctgtccgtgcaccgatgcacacaccgcaccccaccactgtactctgaaattggcgagtgagtggagagccagctctgcggagtcatcacgcagccatggttgtgcctgccgttcatggtggtctttcaggttatcttggcaacatgtacattgcttttattttttttcttttttgctttcattgtacagtcagtactataaaatttctcttttgagttttatacctttgtagcattttagatgacattgtgtttgtactttgttgtgtagagtggaagaattgtgttgaataaacccaagatcggaatgcaaaaaaaaaaaaaaaaaaaaaaaaaaaa

SEQ ID NO: 17 - DNA, 2062 bp, Homo sapiens
gtcagtccctcctgtagccgccgccgccgccgcccgccgcccctctgccagcagctccggcgccacctcgggccggcgtctccggcgggcgggagccaggcgctgacgggcgcggcgggggcggccgagcgctcctgcggctgcgactcaggctccggcgtctgcgcttccccatggggctggcctgcggcgcctgggcgctctgagattgtcactgctgttccaagggcacacgcagagggatttggaattcctggagagttgcctttgtgagaagctggaaatatttctttcaattccatctcttagttttccataggaacatcaagaaatcatgaacaactttggtaatgaagagtttgactgccacttcctcgatgaaggttttactgccaaggacattctggaccagaaaattaatgaagtttcttcttctgatgataaggatgccttctatgtggcagacctgggagacattctaaagaaacatctgaggtggttaaaagctctccctcgtgtcacccccttttatgcagtcaaatgtaatgatagcaaagccatcgtgaagacccttgctgctaccgggacaggatttgactgtgctagcaagactgaaatacagttggtgcagagtctgggggtgcctccagagaggattatctatgcaaatccttgtaaacaagtatctcaaattaagtatgctgctaataatggagtccagatgatgacttttgatagtgaagttgagttgatgaaagttgccagagcacatcccaaagcaaagttggttttgcggattgccactgatgattccaaagcagtctgtcgtctcagtgtgaaattcggtgccacgctcagaaccagcaggctccttttggaacgggcgaaagagctaaatatcgatgttgttggtgtcagcttccatgtaggaagcggctgtaccgatcctgagaccttcgtgcaggcaatctctgatgcccgctgtgtttttgacatgggggctgaggttggtttcagcatgtatctgcttgatattggcggtggctttcctggatctgaggatgtgaaacttaaatttgaagagatcaccggcgtaatcaacccagcgttggacaaatactttccgtcagactctggagtgagaatcatagctgagcccggcagatactatgttgcatcagctttcacgcttgcagttaatatcattgccaagaaaattgtattaaaggaacagacgggctctgatgacgaagatgagtcgagtgagcagacctttatgtattatgtgaatgatggcgtctatggatcatttaattgcatactctatgaccacgcacatgtaaagccccttctgcaaaagagacctaaaccagatgagaagtattattcatccagcatatggggaccaacatgtgatggcctcgatcggattgttgagcgctgtgacctgcctgaaatgcatgtgggtgattggatgctctttgaaaacatgggcgcttacactgttgctgctgcctctacgttcaatggcttccagaggccgacgatctactatgtgatgtcagggcctgcgtggcaactcatgcagcaattccagaaccccgacttcccacccgaagtagaggaacaggatgccagcaccctgcctgtgtcttgtgcctgggagagtgggatgaaacgccacagagcagcctgtgcttcggctagtattaatgtgtagatagcactctggtagctgttaactgcaagtttagcttgaattaagggatttggggggaccatgtaacttaattactgctagttttgaaatgtctttgtaagagtagggtcgccatgatgcagccatatggaagactaggatatgggtcacacttatctgtgttcctatggaaactatttgaatatttgttttatatggatttttattcactcttcagacacgctactcaagagtgcccctcagctgctgaacaagcatttgtagcttgtacaatggcagaatgggccaaaagcttagtgttgtgacctgtttttaaaataaagtatcttgaaataattaggc

SEQ ID NO: 18 - DNA, 2989 bp, Homo sapiens
aattcgggcacgagggtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacagatggagctggaccaccggaccagcggcgggctccacgcctaccccgggccgcggggcgggcaggtggccaagcccaacgtgatcctgcagatcgggaagtgccgggccgagatgctggagcacgtgcggcggacgcaccggcacctgctggccgaggtgtccaagcaggtggagcgcgagctgaaggggctgcaccggtcggtcgggaagctggagagcaacctggacggctacgtgcccacgagcgactcgcagcgctggaagaagtccatcaaggcctgcctgtgccgctgccaggagaccatcgccaacctggagcgctgggtcaagcgcgagatgcacgtgtggcgcgaggtgttctaccgcctggagcgctgggccgaccgcctggagtccacgggcggcaagtacccggtgggcagcgagtcagcccgccacaccgtttccgtgggcgtggggggtcccgagagctactgccacgaggcagacggctacgactacaccgtcagcccctacgccatcaccccgcccccagccgctggcgagctgcccgggcaggagcccgccgaggcccagcagtaccagccgtgggtccccggcgaggacgggcagcccagccccggcgtggacacgcagatcttcgaggaccctcgagagttcctgagccacctagaggagtacttgcggcaggtgggcggctctgaggagtactggctgtcccagatccagaatcacatgaacgggccggccaagaagtggtgggagttcaagcagggctccgtgaagaactgggtggagttcaagaaggagttcctgcagtacagcgagggcacgctgtcccgagaggccatccagcgggagctggacctgccgcagaagcagggcgagccgctggaccagttcctgtggcgcaagcgggacctgtaccagacgctctacgtggacgcggacgaggaggagatcatccagtacgtggtgggcaccctgcagcccaagctcaagcgtttcctgcgccaccccctgcccaagaccctggagcagctcatccagaggggcatggaggtgcaggatgacctggagcaggcggccgagccggccggcccccacctcccggtggaggatgaggcggagaccctcacgcccgcccccaacagcgagtccgtggccagtgaccggacccagcccgagtagagggcatcccggagcccccagcctgcccactacatccagcctgtggctttgcccaccaggacttttgagctggggctgactcctgcaggggaagccctggtccagctgggtgccccctcgagctccgggcggactcgcacacactcgtgtcatccagatgtgagcaccgcacccagcggcaaagagccctcccccctgcagggctccacccatcaccctccctccgtctgtctttccggcctggaccccaccctccacactctcaggccatcacagaacaccccagcttcctcattctgctacaacacccaggccctctggacatccagaaaaccaagtgtccggatggcaggggccagcggccaccaagctcatgggacacccagagcagaagctagggcagagccaatgctgagggagcctcgacttccggcgccgccgccctctcccggcatccgcagagccagctgacgccctccctgcctcccagggcagctggccagcctcgggcagcgcggccccctcctcccaggggagagtagaagtcgcacacgcagcagagcagacctgatgtcccggtgcttcctggcccctcagctccagtgattcacgcccgcctggagaagaatcagagctcagctcatgactcacccatggcaggcggagggtcccagaggggctgagtcctcaaatccggctgaggcagcagctggcaccatcagagccaggagagtgacaacaggtctcaaggttcccacaaagtctttgctgctgtgctgggcaccacccacccctcaccttgcaggctgcctgcgtgggaggcgaagtcccaggacagcccagaggggggctacagagaggagtcggctgcagcagagggcaggagccccagcttagccctgagcgccagcgcgaggaccagggcctgccactaagcccgccccgctggccgccagctgcccgtccccagagccactgcagcaggagtcgggccctgcctccctcccagcagggaaaccccgcccgctgccaggccatcctctctgccagaggctttcatgagccccaaggctggggccacagctcctacccctgcccagcagccctgagctcagctgcaggaaggacatcccagaagccatggctcctggggcgcttccaggcattctgccctgccccgacaccagaaccctggtgctggtgggccactagcgtctgcagcctaagcaggtgctggctcagggttcatcgttctgccttgtccactgggggaccagccctgcagaccactctgacaagtcttcagcccacaccctgccagccccacagattttatttttgcacataagccataaccaatcctcaaggctggcacaggctttggggaagccctggagcctgtgaagaccctggaaacctcatgaggctgtggccaacccctgccccttgccccacacagaccaggccttaaatgtcggtccaggccctgtgcaccttaccccagagacagactctttttgtaagattttgttaataaaacactgaaacttcaaaaaaaaaaaaaaaaaaaaaaaaaaaaaaaaaaaaaaaaaa

SEQ ID NO: 19 - DNA, 2365 bp, Homo sapiens
gaaacggcccgagaagctcgcccggagaacggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatataggaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaatcaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagcgttttgtggagtactggaccaaaaaaataaagcattataaatata

SEQ ID NO: 20 - DNA, 2825 bp, Homo sapiens
gtacggcttccggtggcgggacgcggggccgcgcacgcgggaaaagcttccccggtgtccccccatccccctccccgcgccccccccgcgtccccccagcgcgcccacctctcgcgccggggccctcgcgaggccgcagcctgaggagattcccaacctgctgagcatccgcacacccactcaggagttggggcccagctcccagtttacttggtttcccttgtgcagcctggggctctgcccaggccaccacaggcaggggtcgacatggcagagacactggagttcaacgacgtctatcaggaggtgaaaggttccatgaatgatggtcgactgaggttgagccgtcagggcatcatcttcaagaatagcaagacaggcaaagtggacaacatccaggctggggagttaacagaaggtatctggcgccgtgttgctctgggccatggacttaaactgcttacaaagaatggccatgtctacaagtatgatggcttccgagaatcggagtttgagaaactctctgatttcttcaaaactcactatcgccttgagctaatggagaaggacctttgtgtgaagggctggaactgggggacagtgaaatttggtgggcagctgctttcctttgacattggtgaccagccagtctttgagatacccctcagcaatgtgtcccagtgcaccacaggcaagaatgaggtgacactggaattccaccaaaacgatgacgcagaggtgtctctcatggaggtgcgcttctacgtcccacccacccaggaggatggtgtggaccctgttgaggcctttgcccagaatgtgttgtcaaaggcggatgtaatccaggccacgggagatgccatctgcatcttccgggagctgcagtgtctgactcctcgtggtcgttatgacattcggatctaccccacctttctgcacctgcatggcaagacctttgactacaagatcccctacaccacagtactgcgtctgtttttgttaccccacaaggaccagcgccagatgttctttgtgatcagcctggatcccccaatcaagcaaggccaaactcgctaccacttcctgatcctcctcttctccaaggacgaggacatttcgttgactctgaacatgaacgaggaagaagtggagaagcgctttgagggtcggctcaccaagaacatgtcaggatccctctatgagatggtcagccgggtcatgaaagcactggtaaaccgcaagatcacagtgccaggcaacttccaagggcactcaggggcccagtgcattacctgttcctacaaggcaagctcaggactgctctacccgctggagcggggcttcatctacgtccacaagccacctgtgcacatccgcttcgatgagatctcctttgtcaactttgctcgtggtaccactactactcgttcctttgactttgaaattgagaccaagcagggcactcagtataccttcagcagcattgagagggaggagtacgggaaactgtttgattttgtcaacgcgaaaaagctcaacatcaaaaaccgaggattgaaagagggcatgaacccaagctacgatgaatatgctgactctgatgaggaccagcatgatgcctacttggagaggatgaaggaggaaggcaagatccgggaggagaatgccaatgacagcagcgatgactcaggagaagaaaccgatgagtcattcaacccaggtgaagaggaggaagatgtggcagaggagtttgacagcaacgcctctgccagctcctccagtaatgagggtgacagtgaccgggatgagaagaagcggaaacagctcaaaaaggccaagatggccaaggaccgcaagagccgcaagaagcctgtggaggtgaagaagggcaaagaccccaatgcccccaagaggcccatgtctgcatacatgctgtggctcaatgccagccgagagaagatcaagtcagaccatcctggcatcagcatcacggatctttccaagaaggcaggcgagatctggaagggaatgtccaaagagaagaaagaggagtgggatcgcaaggctgaggatgccaggagggactatgaaaaagccatgaaagaatatgaagggggccgaggcgagtcttctaagagggacaagtcaaagaagaagaagaaagtaaaggtaaagatggaaaagaaatccacgccctctaggggctcatcatccaagtcgtcctcaaggcagctaagcgagagcttcaagagcaaagagtttgtgtctagtgatgagagctcttcgggagagaacaagagcaaaaagaagaggaggaggagcgaggactctgaagaagaagaactagccagtactccccccagctcagaggactcagcgtcaggatccgatgagtagaaacggaggaaggttctctttgcgcttgccttctcacaccccccgactccccacccatattttggtaccagtttctcctcatgaaatgcagtccctggattctgtgccatctgaacatgctctcctgttggtgtgtatgtcactagggcagtggggagacgtcttaactctgctgcttcccaaggatggctgtttataatttggggagagatagggtgggaggcagggcaatgcaggatccaaatcctcatcttactttcccgaccttaaggatgtagctgctgcttgtcctgttcaagttgctggagcaggggtcatgtgaggccaggcctgtagctcctacctggggcctatttctactttcattttgtatttctggtctgtgaaaatgatttaataaagggaactgactttggaaaccaaaaaaa

SEQ ID NO: 21 - DNA, 10488 bp, Homo sapiens
aagagttttcctccgcagctctgagtctccacttttttggtggagaaaggctgcaaaaagaaaaagagacgcagtgagtgggaaaagtatgcatcctattcaaacctaattgaatcgaggagcccagggacacacgccttcaggtttgctcaggggttcatatttggtgcttagacaaattcaaaatgaggaaacatcggcacttgcccttagtggccgtcttttgcctctttctctcaggctttcctacaactcatgcccagcagcagcaagcagatgtcaaaaatggtgcggctgctgatataatatttctagtggattcctcttggaccattggagaggaacatttccaacttgttcgagagtttctatatgatgttgtaaaatccttagctgtgggagaaaatgatttccattttgctctggtccagttcaacggaaacccacataccgagttcctgttaaatacgtatcgtactaaacaagaagtcctttctcatatttccaacatgtcttatattgggggaaccaatcagactggaaaaggattagaatacataatgcaaagccacctcaccaaggctgctggaagccgggccggtgacggagtccctcaggttatcgtagtgttaactgatggacactcgaaggatggccttgctctgccctcagcggaacttaagtctgctgatgttaacgtgtttgcaattggagttgaggatgcagatgaaggagcgttaaaagaaatagcaagtgaaccgctcaatatgcatatgttcaacctagagaattttacctcacttcatgacatagtaggaaacttagtgtcctgtgtgcattcatccgtgagtccagaaagggctggggacacggaaacccttaaagacatcacagcacaagactctgctgacattattttccttattgatggatcaaacaacaccggaagtgtcaatttcgcagtcattctcgacttccttgtaaatctccttgagaaactcccaattggaactcagcagatccgagtgggggtggtccagtttagcgatgagcccagaaccatgttttccttggacacctactccaccaaggcccaggttctgggtgcagtgaaagccctcgggtttgctggtggggagttggccaatatcggcctcgcccttgatttcgtggtggagaaccacttcacccgggcagggggcagccgcgtggaggaaggggttccccaggtgctggtcctcataagtgccgggccttctagtgacgagattcgctacggggtggtagcactgaagcaggctagcgtgttctcattcggccttggagcccaggccgcctccagggcagagcttcagcacatagctaccgatgacaacttggtgtttactgtcccggaattccgtagctttggggacctccaggagaaattactgccgtacattgttggcgtggcccaaaggcacattgtcttgaaaccgccaaccattgtcacacaagtcattgaagtcaacaagagagacatagtcttcctggtggatggctcatctgcactgggactggccaacttcaatgccatccgagacttcattgctaaagtcatccagaggctggaaatcggacaggatcttatccaggtggcagtggcccagtatgcagacactgtgaggcctgaattttatttcaatacccatccaacaaaaagggaagtcataaccgctgtgcggaaaatgaagcccctggacggctcggccctgtacacgggctctgctctagactttgttcgtaacaacctattcacgagttcagccggctaccgggctgccgaggggattcctaagcttttggtgctgatcacaggtggtaagtccctagatgaaatcagccagcctgcccaggagctgaagagaagcagcataatggcctttgccattgggaacaagggtgccgatcaggctgagctggaagagatcgctttcgactcctccctggtgttcatcccagctgagttccgagccgccccattgcaaggcatgctgcctggcttgctggcacctctcaggaccctctctggaacccctgaagttcactcaaacaaaagagatatcatctttcttttggatggatcagccaacgttggaaaaaccaatttcccttatgtgcgcgactttgtaatgaacctagttaacagccttgatattggaaatgacaatattcgtgttggtttagtgcaatttagtgacactcctgtaacggagttctctttaaacacataccagaccaagtcagatatccttggtcatctgaggcagctgcagctccagggaggttcgggcctgaacacaggctcagccctaagctatgtctatgccaaccacttcacggaagctggcggcagcaggatccgtgaacacgtgccgcagctcctgcttctgctcacagctgggcagtctgaggactcctatttgcaagctgccaacgccttgacacgcgcgggcatcctgactttttgtgtgggagctagccaggcgaataaggcagagcttgagcagattgcttttaacccaagcctggtgtatctcatggatgatttcagctccctgccagctttgcctcagcagctgattcagcccctaaccacatatgttagtggaggtgtggaggaagtaccactcgctcagccagagagcaagcgagacattctgttcctctttgacggctcagccaatcttgtgggccagttccctgttgtccgtgactttctctacaagattatcgatgagctcaatgtgaagccagaggggacccgaattgcggtggctcagtacagcgatgatgtcaaggtggagtcccgttttgatgagcaccagagtaagcctgagatcctgaatcttgtgaagagaatgaagatcaagacgggcaaagccctcaacctgggctacgcgctggactatgcacagaggtacatttttgtgaagtctgctggcagccggatcgaggatggagtgcttcagttcctggtgctgctggtcgcaggaaggtcatctgaccgtgtggatgggccagcaagtaacctgaagcagagtggggttgtgcctttcatcttccaagccaagaacgcagaccctgctgagttagagcagatcgtgctgtctccagcgtttatcctggctgcagagtcgcttcccaagattggagatcttcatccacagatagtgaatctcttaaaatcagtgcacaacggagcaccagcaccagtttcaggtgaaaaggacgtggtgtttctgcttgatggctctgagggcgtcaggagcggcttccctctgttgaaagagtttgtccagagagtggtggaaagcctggatgtgggccaggaccgggtccgcgtggccgtggtgcagtacagcgaccggaccaggcccgagttctacctgaattcatacatgaacaagcaggacgtcgtcaacgctgtccgccagctgaccctgctgggagggccgacccccaacaccggggccgccctggagtttgtcctgaggaacatcctggtcagctctgcgggaagcaggataacagaaggtgtgccccagctgctgatcgtcctcacggccgacaggtctggggatgatgtgcggaacccctccgtggtcgtgaagaggggtggggctgtgcccattggcattggcatcgggaacgctgacatcacagagatgcagaccatctccttcatcccggactttgccgtggccattcccacctttcgccagctggggaccgtccaacaggtcatctctgagagggtgacccagctcacccgcgaggagctgagcaggctgcagccggtgttgcagcctctaccgagcccaggtgttggtggcaagagggacgtggtctttctcatcgatgggtcccaaagtgccgggcctgagttccagtacgttcgcaccctcatagagaggctggttgactacctggacgtgggctttgacaccacccgggtggctgtcatccagttcagcgatgaccccaaggcggagttcctgctgaacgcccattccagcaaggatgaagtgcagaacgcggtgcagcggctgaggcccaagggagggcggcagatcaacgtgggcaatgccctggagtacgtgtccaggaacatcttcaagaggcccctggggagccgcattgaagagggcgtcccacagttcctggtcctcatctcgtctggaaagtctgacgatgaggtggtcgtcccggcggtggagctcaagcagtttggcgtggcccctttcacgatcgccaggaacgcagaccaggaggagctggtgaagatctcgctgagccccgaatatgtgttctcggtgagcaccttccgggagctgcccagcctggagcagaaactgctgacgcccatcacgaccctgacctcagagcagatccagaagctcttagccagcactcgctatccacctccagcagttgagagtgatgctgcagacattgtctttctgatcgacagctctgagggagttaggccagatggctttgcacatattcgagattttgttagcaggattgttcgaagactcaacatcggccccagtaaagtgagagttggggtcgtgcagttcagcaatgatgtcttcccagaattctatctgaaaacctacagatcccaggccccggtgctggacgccatacggcgcctgaggctcagaggggggtccccactgaacactggcaaggctctcgaatttgtggcaagaaacctctttgttaagtctgcggggagtcgcatagaagacggggtgccccaacacctggtcctggtcctgggtggaaaatcccaggacgatgtgtccaggttcgcccaggtgatccgttcctcgggcattgtgagtttaggggtaggagaccggaacatcgacagaacagagctgcagaccatcaccaatgaccccagactggtcttcacagtgcgagagttcagagagcttcccaacatagaagaaagaatcatgaactcgtttggaccctccgcagccactcctgcacctccaggggtggacacccctcctccttcacggccagagaagaagaaagcagacattgtgttcctgttggatggttccatcaacttcaggagggacagtttccaggaagtgcttcgttttgtgtctgaaatagtggacacagtttatgaagatggcgactccatccaagtggggcttgtccagtacaactctgaccccactgacgaattcttcctgaaggacttctctaccaagaggcagattattgacgccatcaacaaagtggtctacaaagggggaagacacgccaacactaaggtgggccttgagcacctgcgggtaaaccactttgtgcctgaggcaggcagccgcctggaccagcgggtccctcagattgcctttgtgatcacgggaggaaagtcggtggaagatgcacaggatgtgagcctggccctcacccagaggggggtcaaagtgtttgctgttggagtgaggaatatcgactcggaggaggttggaaagatagcgtccaacagcgccacagcgttccgcgtgggcaacgtccaggagctgtccgaactgagcgagcaagttttggaaactttgcatgatgcgatgcatgaaaccctttgccctggtgtaactgatgctgccaaagcttgtaatctggatgtgattctggggtttgatggttctagagaccagaatgtttttgtggcccagaagggcttcgagtccaaggtggacgccatcttgaacagaatcagccagatgcacagggtcagctgcagcggtggccgctcgcccaccgtgcgtgtgtcagtggtggccaacacgccctcgggcccggtggaggcctttgactttgacgagtaccagccagagatgctcgagaagttccggaacatgcgcagccagcacccctacgtcctcacggaggacaccctgaaggtctacctgaacaagttcagacagtcctcgccggacagcgtgaaggtggtcattcattttactgatggagcagacggagatctggctgatttacacagagcatctgagaacctccgccaagaaggagtccgtgccttgatcctggtgggccttgaacgagtggtcaacttggagcggctaatgcatctggagtttgggcgagggtttatgtatgacaggcccctgaggcttaacttgctggacttggattatgaactagcggagcagcttgacaacattgccgagaaagcttgctgtggggttccctgcaagtgctctgggcagaggggagaccgcgggcccatcggcagcatcgggccaaagggtattcctggagaagacggctaccgaggctatcctggtgatgagggtggacccggtgagcgtggtccgcctggtgtgaacggcactcaaggtttccagggctgcccgggccagagaggagtaaagggctctcggggattcccaggagagaagggcgaagtaggagaaattggactggatggtctggatggtgaagatggagacaaaggattgcctggttcttctggagagaaagggaatcctggaagaaggggtgataaaggacctcgaggagagaaaggagaaagaggagatgttgggattcgaggggacccgggtaacccaggacaagacagccaggagagaggacccaaaggagaaaccggtgacctcggccccatgggtgtcccagggagagatggagtacctggaggacctggagaaactgggaagaatggtggctttggccgaaggggaccccccggagctaagggcaacaagggcggtcctggccagccgggctttgagggagagcaggggaccagaggtgcacagggcccagctggtcctgctggtcctccagggctgataggagaacaaggcatttctggacctaggggaagcggaggtgcccgtggcgctcctggagaacgaggcagaaccggtccactgggaagaaagggtgagcccggagagccaggaccaaaaggaggaatcgggaacccgggccctcgtggggagacgggagatgacgggagagacggagttggcagtgaaggacgcagaggcaaaaaaggagaaagaggatttcctggatacccaggaccaaagggtaacccaggtgaacctgggctaaatggaacaacaggacccaaaggcatcagaggccgaaggggaaattcgggacctccagggatagttggacagaaggggagacctggctacccaggaccagctggtccaaggggcaacaggggcgactccatcgatcaatgtgccctcatccaaagcatcaaagataaatgcccttgctgttacgggcccctggagtgccccgtcttcccaacagaactagcctttgctttagacacctctgagggagtcaaccaagacactttcggccggatgcgagatgtggtcttgagtattgtgaatgtcctgaccattgctgagagcaactgcccgacgggggcccgggtggctgtggtcacctacaacaacgaggtgaccacggagatccggtttgctgactccaagaggaagtcggtcctcctggacaagattaagaaccttcaggtggctctgacatccaaacagcagagtctggagactgccatgtcgtttgtggccaggaacacatttaagcgtgtgaggaacggattcctaatgaggaaagtggctgttttcttcagcaacacacccacaagagcatccccacagctcagagaggctgtgctcaaactctcagatgcggggatcacccccttgttccttacaaggcaggaagaccggcagctcatcaacgctttgcagatcaataacacagcagtggggcatgcgcttgtcctgcctgcagggagagacctcacagacttcctggagaatgtcctcacgtgtcatgtttgcttggacatctgcaacatcgacccatcctgtggatttggcagttggaggccttccttcagggacaggagagcggcagggagtgatgtggacatcgacatggctttcatcttagacagcgctgagaccaccaccctgttccagttcaatgagatgaagaagtacatagcgtacctggtcagacaactggacatgagcccagatcccaaggcctcccagcacttcgccagagtggcagttgtgcagcacgcgccctctgagtccgtggacaatgccagcatgccacctgtgaaggtggaattctccctgactgactatggctccaaggagaagctggtggacttcctcagcaggggaatgacacagttgcagggaaccagggccttaggcagtgccattgaatacaccatagagaatgtctttgaaagtgccccaaacccacgggacctgaaaattgtggtcctgatgctgacgggcgaggtgccggagcagcagctggaggaggcccagagagtcatcctgcaggccaaatgcaagggctacttcttcgtggtcctgggcattggcaggaaggtgaacatcaaggaggtatacaccttcgccagtgagccaaacgacgtcttcttcaaattagtggacaagtccaccgagctcaacgaggagcctttgatgcgcttcgggaggctgttgccgtccttcgtcagcagtgaaaatgctttttacttgtccccagatatcaggaaacagtgtgattggttccaaggggaccaacccacaaagaaccttgtgaagtttggtcacaaacaagtaaatgttccgaataacgttacttcaagtcctacatccaacccagtgacgacaacgaagccggtgactacgacgaagccggtgaccaccacaacaaagcctgtaaccaccacaacaaagcctgtgactattataaatcagccatctgtgaagccagccgctgcaaagccggcccctgcgaaacctgtggctgccaagcctgtggccacaaagacggccactgttagacccccagtggcggtgaagccagcaacagcagcgaagcctgtagcagcaaagccagcagctgtaagaccccccgctgctgctgcaaaaccagtggcgaccaagcctgaggtccctaggccacaggcagccaaaccagctgccaccaagccagccaccactaagcccgtggttaagatgctccgtgaagtccaggtgtttgagataacagagaacagcgccaaactccactgggagaggcctgagccccccggtccttatttttatgacctcaccgtcacctcagcccatgatcagtccctggttctgaagcagaacctcacggtcacggaccgcgtcattggaggcctgctcgctgggcagacataccatgtggctgtggtctgctacctgaggtctcaggtcagagccacctaccacggaagtttcagtacaaagaaatctcagcccccacctccacagccagcaaggtcagcttctagttcaaccatcaatctaatggtgagcacagaaccattggctctcactgaaacagatatatgcaagttgccgaaagacgaaggaacttgcagggatttcatattaaaatggtactatgatccaaacaccaaaagctgtgcaagattctggtatggaggttgtggtggaaacgaaaacaaatttggatcacagaaagaatgtgaaaaggtttgcgctcctgtgctcgccaaacccggagtcatcagtgtgatgggaacctaagcgtgggtggccaacatcatatacctcttgaagaagaaggagtcagccatcgccaacttgtctctgtagaagctccgggtgtagattcccttgcactgtatcatttcatgctttgatttacactcgaactcgggagggaacatcctgctgcatgacctatcagtatggtgctaatgtgtctgtggaccctcgctctctgtctccagcagttctctcgaatactttgaatgttgtgtaacagttagccactgctggtgtttatgtgaacattcctatcaatccaaattccctctggagtttcatgttatgcctgttgcaggcaaatgtaaagtctagaaaataatgcaaatgtcacggctactctatatacttttgcttggttcattttttttcccttttagttaagcatgactttagatgggaagcctgtgtatcgtggagaaacaagagaccaactttttcattccctgcccccaatttcccagactagatttcaagctaattttctttttctgaagcctctaacaaatgatctagttcagaaggaagcaaaatcccttaatctatgtgcaccgttgggaccaatgccttaattaaagaatttaaaaaagttgtaatagagaatatttttggcattcctctcaatgttgtgtgttttttttttttgtgtgctggagggaggggatttaattttaattttaaaatgtttaggaaatttatacaaagaaactttttaataaagtatattgaaagtttaaaaaaaaaaaaaaaa

SEQ ID NO: 22 - DNA, 1044 bp, Homo sapiens
gaattccctgaggaggcgaatccggcgggtatcagagccatcagaaccgccaccatgacggtgggcaagagcagcaagatgctgcagcatattgattacaggatgaggtgcatcctgcaggacggccggatcttcattggcaccttcaaggcttttgacaagcacatgaatttgatcctctgtgactgtgatgagttcagaaagatcaagccaaagaactccaaacaagcagaaagggaagagaagcgagtcctcggtctggtgctgctgcgaggggagaatctggtctcaatgacagtagagggacctcctcccaaagatactggtattgctcgagttccacttgctggagctgccgggggcccagggatcggcagggctgctggcagaggaatcccagctggggttcccatgccccaggctcctgcaggacttgctgggccagtccgtggggttggcgggccatcccaacaggtgatgaccccacaaggaagaggtactgttgcagccgctgcagctgctgccacagccagtattgccggggctccaacccagtacccacctggccgtgggggtcctcccccacctatgggccgaggagcaccccctccaggcatgatgggcccacctcctggtatgagacctcctatgggtcccccaatggggatcccccctggaagagggactccaatgggcatgccccctccgggaatgcggcctcctccccctgggatgcgaggccttctttgacccttggccacagagtatggaagtagctccgcagaggcgtgggctcgattcctcagggccacgttaccacagacctgtttgtttcttatgctgttgttcgtggagtctcatgggattgtctggtttcccttacagggccccctcccccgggaatgcgcccaccaaggccctagactcatcttggccctcctcagctccctgcctgtttcccgtaaggctgtacatagtccttttatctccttgtggcctatgaaactggtttataataaactcttaagagaacattataattgc

SEQ ID NO: 23 - DNA, 1475 bp, Homo sapiens
gtcgacgcggccgcgctccgctcccgtgagtaacttggctccgggggctccgctcgcctgcccgcacgccgcccgccacccaggaccgcgccgccggcctccgccgctagcaaacccttccgacggccctcgctgcgcaagccgggacgcctctcccccctccgcccccgccgcggaaagttaagtttgaagaggggggaagaggggaacatggacatgaagaggaggatccacctggagctgaggaaccggaccccggcagctgttcgagaacttgtcttggacaattgcaaatcaaatgatggaaaaattgagggcttaacagctgaatttgtgaacttagagttcctcagtttaataaatgtaggcttgatctcagtttcaaatctccccaagctgcctaaattgaaaaagcttgaactcagtgaaaatagaatctttggaggtctggacatgttagctgaaaaacttccaaatctcacacatctaaacttaagtggaaataaactgaaagatatcagcaccttggaacctttgaaaaagttagaatgtctgaaaagcctggacctctttaactgtgaggttaccaacctgaatgactaccgagagagtgtcttcaagctcctgccccagcttacctacttggatggctatgaccgagaggaccaggaagcacctgactcagatgccgaggtggatggtgtggatgaagaggaggaggacgaagaaggagaagatgaggaagacgaggacgatgaggatggtgaagaagaggagtttgatgaagaagatgatgaagatgaagatgtagaaggggatgaggacgacgatgaagtcagtgaggaggaagaagaatttggacttgatgaagaagatgaagatgaggatgaggatgaagaggaggaagaaggtgggaaaggtgaaaagaggaagagagaaacagatgatgaaggagaagatgattaagaccccagatgacctgcagaaacagaactgttcagtattggttggactgctcatggattttgtagctgtttaaaaaaaaaaaaaaggtagctgtgatacaaaccccaggacacccacccacccaaagagccaaagaatagttcctgtgacattccgccttccttccatgtagtccctcttggtaatctaccaccaagcttgtggacttcaccccaacaaaattgtaagcgttgttaggtttttgtgtaagattcttgctgtagcgtggatagctgtgattggtgagtcaaccgtctgtggctaccagttacactgagattgtaacagcatttttactttctgtacaacaaaaaagctttgtaaataaaatcttaacattttgggtctgttttttcatgctttgctttttaattattattattattttttttacattaggacattttatgtgacaactgccaaaaaagtatttttaagaatttaagcgaaataaacagttactctttggc

SEQ ID NO: 24 - DNA, 2690 bp, Homo sapiens
gctcttttctcgggacgggagaggccgtgtagcgtcgccgttactccgaggagataccagtcggtagaggagaagtcgaggttagagggaactgggaggcactttgctgtctgcaatcgaagttgagggtgcaaaaatgcagagtaataaaacttttaacttggagaagcaaaaccatactccaagaaagcatcatcaacatcaccaccagcagcagcaccaccagcagcaacagcagcagccgccaccaccgccaatacctgcaaatgggcaacaggccagcagccaaaatgaaggcttgactattgacctgaagaattttagaaaaccaggagagaagaccttcacccaacgaagccgtctttttgtgggaaatcttcctcccgacatcactgaggaagaaatgaggaaactatttgagaaatatggaaaggcaggcgaagtcttcattcataaggataaaggatttggctttatccgcttggaaacccgaaccctagcggagattgccaaagtggagctggacaatatgccactccgtggaaagcagctgcgtgtgcgctttgcctgccatagtgcatcccttacagttcgaaaccttcctcagtatgtgtccaacgaactgctggaagaagccttttctgtgtttggccaggtagagagggctgtagtcattgtggatgatcgaggaaggccctcaggaaaaggcattgttgagttctcagggaagccagctgctcggaaagctctggacagatgcagtgaaggctccttcctgctaaccacatttcctcgtcctgtgactgtggagcccatggaccagttagatgatgaagagggacttccagagaagctggttataaaaaaccagcaatttcacaaggaacgagagcagccacccagatttgcacagcctggctcctttgagtatgaatatgccatgcgctggaaggcactcattgagatggagaagcagcagcaggaccaagtggaccgcaacatcaaggaggctcgtgagaagctggagatggagatggaagctgcacgccatgagcaccaggtcatgctaatgagacaggatttgatgaggcgccaagaagaacttcggaggatggaagagctgcacaaccaagaggtgcaaaaacgaaagcaactggagctcaggcaggaggaagagcgcaggcgccgtgaagaagagatgcggcggcacgaagaagaaatgatgcggcgacacgaggaaggattcaagggaaccttccctgatgcgagagagcaggagattcggatgggtcagatggctatgggaggtgctatgggcataaacaacagaggtgccatgccccctgctcctgtgccagctggtaccccagctcctccaggacctgccactatgatgccggatggaactttgggattgaccccaccaacaactgaacgctttggtcaggctgctacaatggaaggaattggggcaattggtggaactcctcctgcattcaaccgtgcagctcctggagctgaatttgccccaaacaaacgtcgccgatactaataagttgcagtgtctagtttctcaaaacccttaaaagaaggaccctttttggactagccagaattctaccctggaaaagtgttagggattccttccaatagttagatctaccctgcctgtactactctaagggattccttccaatagttagatctaccctgcctgtactactctagggagtatgctggaggcagagggcaagggaggggtggtattaaacaatgcaattctgtgtggtatattgtttaatcagttctgtgtggtgcattcctgaagtctctaatgtgactgttgagggcctggggaaaccatggcaaagtggatccagttagagcccattaatcttgatcattccggttttttttttttttgtccatcttgtttcatttgcttgccccgcccccgagacggagtcttactctgtcgcccaggctggagtgtagtggcatgatctcggctcactgcaatctctgcctcccgggttcaagcttgtccaggttgatcttgaactcctgacctcgtgatctacccacctcggtctcccaaaatgctgggattacaggggtgagccaccgtgcccaacctcacttgcttcttatccttacactcccccagccccagagaaactgccacatacaccacaaaaaccaaacatgccccaatgaccttagccccattgctccattcactcccaggtgagaattcaggcaaacgtccacaaaggtcacaggcagcgtacatacggttctgttataccccatatattaccccttcatgtcctaaagaagacattttctcttagagattttcattttagtgtatctttaaaaaaaaaatcttgtgttaacttgcctccatctttttcttggggtgagggacaccagggaatgacccttttgtgtctatgatgttgctgttcacagcttttcttgataggcctagtacaatcttgggaacagggttactgtatactgaaggtctgacagtagctcttagactcgcctatcttaggtagtcatgctgtgcattttttttttcattggtgtactgtgtttgatttgtctcatatatttggagtttttctgaaaaatggagcagtaatgcagcatcaacctattaaaatacttttaagcctttt

SEQ ID NO: 25 - DNA, 1828 bp, Homo sapiens
cagttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactc

SEQ ID NO: 26 - DNA, 500 bp, Homo sapiens
gctctcagaggcagcgtgcgggtgtgctctttgtgaaattccaccatggcgtaccgtggccagggtcagaaagtgcagaaggttatggtgcagcccatcaacctcatcttcagatacttacaaaatagatcgcggattcaggtgtggctctatgagcaagtgaatatgcggatagaaggctgtatcattggttttgatgagtatatgaaccttgtattagatgatgcagaagagattcattctaaaacaaagtcaagaaaacaactgggtcggatcatgctaaaaggagataatattactctgctacaaagtgtctccaactagaaatgatcaatgaagtgagaaattgttgagaaggatacagtttgtttttagatgtcctttgtccaatgtgaacatttattcatattgttttgattaccctcgtgttactacaagatggcaataaatactatgggattgtttgtattaaaaaatttacattgcttcttaaaaaaaaa

SEQ ID NO: 27 - DNA, 4661 bp, Homo sapiens
gctggacttgcctgcggtgacacctgctcccctctgagagcttcaggttctccggcctgccttcactggtttgtgtccagagccggactgattctctcaatttgcgatcttcagcctgttaaacaagaaaacgaaaaaccccttccagaaaacatggatgcatttgaaaaagtgagaacaaaattagaaacacagccacaagaagaatatgaaatcatcaatgtggaagttaaacatggtggttttgtttattaccaagaaggttgttgcttggttcgttccaaagatgaagaagcagacaatgataattatgaagttttattcaatttggaggaacttaagttagaccagcccttcattgattgtatcagagttgctccagatgaaaaatatgtggctgccaagataagaactgaagattctgaagcatctacctgtgtaattataaagctcagcgatcagcccgtaatggaagcttctttcccgaatgtgtccagttttgaatgggtaaaggacgaggaagatgaagatgttttattctacaccttccagaggaaccttcgctgtcatgacgtatatcgagccacttttggtgataacaaacgtaatgaacgcttttacacagaaaaagacccaagctactttgttttcctttatcttacaaaagacagtcgtttcctcaccataaatattatgaacaagactacttctgaagtgtggttgatagatggcctgagcccttgggacccaccagtacttatccagaagcgaatacatggggtcctttactatgttgaacacagagatgatgaattatacattctcactaatgttggagaacctacagaatttaagctaatgagaacagcggctgatacccctgcaattatgaattgggatttattttttacaatgaagagaaatacaaaagtgatagacttggacatgtttaaggatcactgtgttctatttctgaagcacagcaatctcctttatgttaatgtgattggtctggctgatgattcagttcggtctctaaagctccctccttgggcctgtggattcataatggatacaaattctgacccaaagaactgcccctttcaactttgctctccaatacgtcccccaaaatattacacatacaagtttgcagaaggcaaactgtttgaggaaactgggcatgaagacccaatcacaaagactagtcgcgttttacgtctagaagccaaaagcaaggatggaaaattagtgccaatgactgttttccacaaaactgactctgaggacttgcagaagaaacctctcttggtacatgtatatggagcttatggaatggatttgaaaatgaatttcaggcctgagaggcgggtcctggtggatgatggatggatattagcatactgccatgttcgaggtggtggtgagttaggcctccagtggcacgctgatggccgcctaactaaaaaactcaatggccttgctgatttagaggcttgcattaagacgcttcatggccaaggcttttctcagccaagtctaacaaccctgactgctttcagtgctggaggggtgcttgcaggagcattgtgtaattctaatccagagctggtgagagcggtgactttggaggcacctttcttggatgttctcaacaccatgatggacactacacttcctctgacattagaagaattagaagaatgggggaatccttcatctgatgaaaaacacaagaactacataaaacgttactgtccctatcaaaatattaaacctcagcattatccttcaattcacataacggcatatgaaaacgatgaacgggtacctctgaaaggaattgtaagttatactgagaaactcaaggaagccatcgcggagcatgctaaggacacaggtgaaggctatcagacccctaatattattctagatattcagcctggaggcaatcatgtaattgaggattctcacaaaaagattacagcccaaattaaattcctgtacgaggaacttggacttgacagcaccagtgttttcgaggatcttaagaaatacctgaaattctgaaacactgcattcaactgggaattggaaacacactgaaatatttcatagtcttacttccaattgagttagcaaaaaaaaaattaataacttgagacttttaagttattaattttttaaaatgtgcttctccatctaaattttgcttagtctacatctcacttgcttatactattcctccattgatgcacatgcccattaacctaggaaagtagttttcaaatcatgctccttagaaggatgtggagtagagggaagggaaggattggtgatagcagagctccaggcctcccttccagtcagaacagttgagcagtttacaaattagtgtcctgcctctttgctagcaaatgcttttagacactgtggcagtgagtcatcctctaatttctatgactgcattttaagggaaaagataaaattcttccccttaaaattcgttaaagtttttgaataatctggggtcctaatgtgttctggtcatccctgattgatgctatctgaataaagttataagctcctataagccataatttacttttaaacattttatttttttcaaaacatttgagaacctttcttaaagcggttacattcaagctacagaaatatcgaagaattaatgattgttcaccaagcagcatgctgtacatgaagctattacaaatgcttacaatcccactgaaatgccagtgtcttcatctcttcataaaggtgcctaacacgaggtatacagtatgttcagtacactggaatagcatgctcgattggaaacaaagcatctatctctgaaagctgtttggcgatgaaggagattcttcgtgttgtgttcaaagatgagtccctctcccttgtccagaaaaatgccacttgtatcaactttactgcctttgtcggcagaattggtacttaaccttattcttattttagcgggaaggcccgaaatcatattatgtagatttaacagtgttgattctccaaaattcagaaccacgataaagattctgtcgatgccatccagctctcttgtgtacacaacatagtggctgtcattcctcaaatggcaaaaccagcccctgttgaggagtagctcattggcatgaagtagacttaaatcttgatataacttcaaagccgatctgggctgagtcttttggacctatttttttaaaaaagtatttacgtaagtgtttgattctaagaattgtttgtaagtatttttaatatattgtaaggagttatttacccaaaacacttgctccaattttgccccttataattgccaaattgtaagcatcaataagtaggtaagaacaatttatataaaaactgatagaaatgacaaattcggggtttcggcttgtccgggagtcaataagtacgcacagtgctctgctacattgtagagtttctgtagagatcaaatttgactccactttaggagtcccaaagcaaatgtccatgtctaagatgaatatttaacttgcatagtcattctgtgctatattgtaactgccagatggccagaaagaaggcaacagtggactcagacttctgaggaatttgggtttgttcccctttgtagactaatgtgtaggttgctgttgtgcgaagatcgtgtaactttagcagacatgtatttcttgcacagctaatagaagacaaagttgaaaaaaaggatgcaaaataaaaagctgcctaaggtgaaagttagaaattgtagacttttttttaccataatagtatgtgttcattgaagatgatttgggtttattttacagctatataaaacataatttgatgatgtacttctaacctttcaagcattttctgttattgactatataatatagcctccataaatgtttttaatgacaatattctgttgaacggttgtaccatactcagccatgccctttcattttgacgatagtgtttctaatattttgtatttttattcccctccccccatttttgtattacttaagatagattatcagaaagacagttactttgtcaaagagtatgggcacttgatacataatgccaaattattcttcataagagctgttgccaaatcagtgataatgttcatttaattgtattcttgccagccatgtttactggggtgatagttgttattgtggttgttattgttctttaggggtaggttcccaatatgtggtctttaaataattatctaatggtgtttaaaaagatgtttattctgtttgtcaggtacaaagatatttatgatacatgtatgacttgtctaagttattaacattttctctagccttaggtaatgcatgaaagcacatgtttcagtgccactcacataagaagtgcccggtaagtgttagctattattgtctacttgagttactactttctaaaagtatgttgaagtctttttctgtaattgcagatttgttgattttgcatttgagtattttctatattttgaagctgttagatgcatagtcatgatttttggtggaatgttttatcaatttttgaaaattgcctttgtctcatataatgcttttcatattgaactatattttgtctgctattaaatacttccaagcctg

SEQ ID NO: 28 - DNA, 1135 bp, Homo sapiens
ggatccggcaacgaaggtaccatggccggactccggagccgcacaaaccagggctcgccatgaagccaggattcagtccccgtgggggtggctttggcggccgagggggctttggtgaccgtggtggtcgtggaggccgagggggctttggcgggggccgaggtcgaggcggaggctttagaggtcgtggacgaggaggaggtggaggcggcggcggcggtggaggaggaggaagaggtggtggaggcttccattctggtggcaaccggggtcgtggtcggggaggaaaaagaggaaaccagtcggggaagaatgtgatggtggagccgcatcggcatgagggtgtcttcatttgtcgaggaaaggaagatgcactggtcaccaagaacctggtccctggggaatcagtttatggagagaagagagtctcgatttcggaaggagatgacaaaattgagtaccgagcctggaaccccttccgctccaagctagcagcagcaatcctgggtggtgtggaccagatccacatcaaaccgggggctaaggttctctacctcggggctgcctcgggcaccacggtctcccatgtctctgacatcgttggtccggatggtctagtctatgcagtcgagttctcccaccgctctggccgtgacctcattaacttggccaagaagaggaccaacatcattcctgtgatcgaggatgctcgacacccacacaaataccgcatgctcatcgcaatggtggatgtgatctttgctgatgtggcccagccagaccagacccggattgtggccctgaatgcccacaccttcctgcgtaatggaggacactttgtgatttccattaaggccaactgcattgactccacagcctcagccgaggccgtgtttgcctccgaagtgaaaaagatgcaacaggagaacatgaagccgcaggagcagttgacccttgagccatatgaaagagaccatgccgtggtcgtgggagtgtacaggccaccccccaaggtgaagaactgaagttcagcgctgtcaggattgcgagagatgtgtgttgatactgttgcacgtgtgtttttctattaaaagactcatccgtcaaaaaaaaaaaaaaaaaaaaaaaaaaaaaa

SEQ ID NO: 29 - DNA, 6734 bp, Homo sapiens
cccagttgtctgcgggctgcggggagctaagtccccagattggaggaggctggctctggtcttcgatgcacaggagtggccgttatggaacgcagcagcagcgtgcagggtcaaagacagccggccccccatgtcagtggtctaggatggccagtgaaggcaccaacatcccaagtcctgtggtgcgccagattgacaagcagtttctgatttgcagtatatgcctggaacggtacaagaatcccaaggttctcccctgtctgcacactttctgcgagaggtgcctgcagaactacattcctgcccacagtttaaccctctcctgcccagtgtgccgccagacctccatcctgcccgagaaaggggtggccgcgctccagaacaatttcttcatcacaaacctgatggacgtgctgcagcgaactccaggcagcaacgctgaggagtcttccatcctggagacagtcactgctgtggctgcgggaaagcctctctcttgcccaaaccacgatgggaatgtgatggaattttactgccagtcctgtgagactgccatgtgtcgggagtgcacggagggggagcacgcagagcaccccacagttccactcaaggatgtggtggaacagcacaaggcctcgctccaggtccagctggatgctgtcaacaaaaggctcccagaaatagattctgctcttcagttcatctctgaaatcattcatcagttaaccaaccaaaaggccagcatcgtggatgacattcattccacctttgatgagctccagaagactttaaatgtgcgcaagagtgtgctgcttatggaattggaggtcaactatggcctcaaacacaaagtcctccagtcgcagctggatactctgctccaggggcaggagagcattaagagctgcagcaacttcacagcgcaggccctcaaccatggcacggagaccgaggtcctactggtgaagaagcagatgagcgagaagctgaacgagctggccgaccaggacttccccttgcacccgcgggagaacgaccagctggatttcatcgtggaaaccgaggggctgaagaagtccatccacaacctcgggacgatcttaaccaccaacgccgttgcctcagagacagtggccacgggcgaggggctgcggcagaccatcatcgggcagcccatgtccgtcaccatcaccaccaaggacaaagacggtgagctgtgcaaaaccggcaacgcctacctcaccgccgaactgagcacccccgacgggagcgtggcagacggggagatcctggacaacaagaacggcacctatgagtttttgtacactgtccagaaggaaggggactttaccctgtctctgagactctatgaccagcacatccgaggcagcccgtttaagctgaaagtgatccgatccgctgatgtgtctcccaccacagaaggcgtgaagaggcgcgttaagtccccggggagcggccacgtcaagcagaaagctgtgaaaagacccgcaagcatgtacagcactggaaaacgaaaagagaatcccatcgaagacgatttgatctttcgagtgggtaccaaaggaagaaataaaggagagtttacaaatcttcagggggtagctgcatctacaaatggaaagatattaattgcagacagtaacaaccaatgtgtgcagatattttccaatgatggccagttcaaaagtcgttttggcatacggggacgctctccggggcagctgcagcggcccacaggagtggctgtacatcccagtggggacataatcattgccgattatgataataaatgggtcagcattttctcctccgatgggaaatttaagacaaaaattggatcaggaaagctgatgggacccaaaggagtttctgtggaccgcaatgggcacattattgttgtggacaacaaggcgtgctgcgtgtttatcttccagccaaacgggaaaatagtcaccaggtttggtagccgaggaaatggggacaggcagtttgcaggtccccattttgcagctgtaaatagcaataatgagattattattacagatttccataatcattctgtcaaggtgtttaatcaggaaggagaattcatgttgaagtttggctcaaatggagaaggaaatgggcagtttaatgctccaacaggtgtagcagtggattcaaatggaaacatcattgtggccgactggggaaacagcaggatccaggtttttgatgggagtggatcatttttgtcctacattaacacatctgctgacccactctatggcccccaaggcctggccctaacttcagatggtcatgttgtggttgcagactctggaaatcactgtttcaaagtctatcgatacttacagtaatggtgggcaggtggatacccgcttccatggtcttgcactataaactggaatggatttctcaatgcgggaccagattatgactagagtttttatgccagaaggaatcattggtgaactttccaaggttatttctgaatgtaacaatttccttaaaaatgacttatccaatttctgtatttcacctttagggttaaaaaaaactcttctactgaatctataaaaactgcagttttacatctgtgaactatggcttaagggacaggatttatgtagctaaactaattttgcaaatcaaacagacacttaaaaaaactagcatatgtaaaggtattcgttaatcctgtgaatggtagcttttgcacagaacttccaaaagcaaaacaaaaacaaaatctattgtagttatatacttcatttaacctaggtcacaagacccagggaatcttctaacctcacttttacagtaggtattactcttgtgacatttttttggttatcaacaactaaatataaattactttggaaaaagtaaggctgtcttgcaaaatgatcccagctctgattagcagccctctggagttcagaacttaagtatcagtgcaaatttctcaacctttctgggttagacaaagatccttttttgtgtgttcttttcaccacccctttggctcaccttgtatcagcaaacaaagtacttcttcagggaaacctgaaatttctaatgccttgaaaagcatattacaaaagtaatgctaccttttgggaaacaaactgccccgttaactccagatcattgcactggaatgtaatcaagaaagttagtcatgttttatgtaccatgttttcacacgtgtctcttctcttcgacttcctgaaagcgaaagctttacctcctgcaaatgtcagcacatgtagtaggacaccagtatcctaggacagagagccataagtagccctttggaggactgatggtgtcaaccaaaggcatgtgattgattaatgattcccccttagaaagcaagtgttaccaaagttgtgttatcttgaaagcattacaggtaagggcatgttatggttatttatcattgtttaatgaatagtagaggtgtcaagggactatgtatacatgattagggtaagatagaatgtattatatatatatatatatatacacacacacatatatatagctgaatctttggtgtattgaaataggcagcactctgaaagacagaagcttcgtccagccactcttcagcacattcctttactaagcagtttaaagccgtcctagtggagcaagccctaaagcagatttaatttttgccattttccaagaatgacggtggtggcttttagtcagaaaatggccttctgtgctttcaaaaaaaaaacaaaaaaaaaaccacacacacacataaaaaacccaacaggtcaaaataaaagttgaacttgagttacatttaatttaaatataaatgcattttgagaaatgttaagaacaatttagtcaatcgttcatctgtcattggtactgtaaaataagctgtggtctatttccactgtttaattttctactcagttctaccaaataggatgtcatgtttgacatttttgatagtgactttggggtcttcttcactgaaagcaccttagaactgtactataagaaaacatttcccctatgtataattatatgaatgtgatgtttattgcttattaatttataattcagtcattctctatataggacttcttaaaatttagaagggaaatctagctacttcaaattgtctgttaaatttattatgcccaaatcaacctctgaaaaaaggtttttccaggaagatttacatttaggtttaatatttttttagttaggtagagttttaaaaaatacttgagcctgtccgtgataaagctataaaattcaataactttttagaatgttaaatgaagacactgtttcctaacatcagtgagatacatctttgaatttaaacattcatatttactgagtacctactaggtaccaagtactcttttaggcactggaaatacagtgatggacaaaacaggtaaaaaatcgctgccccctcagagctgacattctggggtgggaatttcattttgccacgtactaacgttctgcacaaaagacaggctagactcttgtctagattgtttaaaagaaacttttcaaattggttacattaattttagtttattttcacaagtaaaaatggctttttatttagattctttctgtcccaggctgttgatcttaaaactagttgatttaaagagtttttttgcacaacatttcaattatatttgtgaacttagaaattaacttacaatctaaccagccatcatatcatatcctatcaggctagatatctcaatagtagactgaatacaaagctaattttttttacatgtcaatattggcacaaactggaatgaaagaatagtttgattcagacctgctccactatgtgttgctaaaacacatgctatgagcactcgaggaaacactatattttttccaaaaaatatgtgattatatatgttaaagtatagataacatttcacacttggatacatatgtgcatttactgtatttcttggtaagcatatttttgggggaaagtgctgctgatatgatacaagtagacaaaatttaaatgaaattttgtcacattctatggaaaatggtttctggtaaactgagaaggatattaaaataagtggcttttttctgggctaccattattgtttgatttctctttgtcaagtgtatagaacctgtcatacattcatgataagtagcactgaaaaattactcattcaaatttcccctgggcacgtaaggcaaaatattgccggttgggatttcaaggtcagtgacgacgcatttcctcccagtacagaccccccagccccccttgctggacatggggaggcagagagtcacttgaccatccagaaatacatgactacaagtcctttatgactgtttgccattttttttaatggtacttagtattttgatcaaactttagtctccagaactaaacaagtccctaagtttccttattttaatttactgtgactagatttgaagcaaataaatactccagatccatgcagctagaacacacttgcttccactactaaatatacagggtatgtcctaacatggagttaactggaatagcagtacactagcaagtatctgtgaatccttagcactgacgggttaacagaaatgctttggtaatacctacttagttaattggaggaagtagtaaataaacattaggtaatctgcagattacttcaaatgggaaaaatctttttgtagactctatagtaccctctctattcactagcttctgaaaagggaggagtatttttagtttgacaatttaataatttaaaaacaagacatctccaggtaggaaaaaatgaaagctatttcatgcaaacattatctaatttagcttaaaagtgaaagtggtaatactgttggtttctgtaaatgttgcagggttttaaactttataattactttaatatttttgataactagaaatctagtattgccataaaggaaactaagtgcccatcaaagatttgtttggtataaataaagaattatttgttttgttttcaatgacagtaagctacaaatcatgatgcttaaaaactttctaaagatgaattgtgtggcagtgattggtctgtttgtggagaatgtatgaaagctattaatattctagaatagattaataaattggctatgttgttccaatgaatgtacagcacttccattaacttttgaaagcaacacagccttaaactcaatgcttttgctttatgacatgggaatgttctgtcatcaatggagtgtattcttgtaatagaattctttatatcgttctcaattctatagactttcaagcctatgtatgaatatgaaggggtttttttttttttgctttgttttctttttagattttgtacattccatctttataggtctgtttcatatgttttatgtatagaacactaagtcttgcactctctgacattgatactgatatattctcgtcatttgttcttttatgaatcaaaatgttgactgcctatttaaagaaaagaatgaacgctgtgcatcaaagtgtttgtatgttcgtagctacatacgtaccacagtattttggatgctttagtctacaatgaaactttcaattaattctgtcttgaaacataggagaaacaggattcatgtgtatctctttaccatgcacaaaatctcaaatcattataataaagcttgttttctcc

SEQ ID NO: 30 - DNA, 3744 bp, Homo sapiens
ccacgcgtccggtggcggtcgagcgtggcgtaggcgaatcctcggcactaagcatatggacctcgcggcggcagcggagccgggcgccggcagccagcacctggaggtccgcgacgaggtggccgagaagtgccagaaactgttcctggacttcttggaggagtttcagagcagcgatggagaaattaaatacttgcaattagcagaggaactgattcgtcctgagagaaacacattggttgtgagttttgtggacctggaacaatttaaccagcaactttccaccaccattcaagaggagttctatagagtttacccttacctgtgtcgggccttgaaaacattcgtcaaagaccgtaaagagatccctcttgccaaggatttttatgttgcattccaagacctgcctaccagacacaagattcgagagctcacctcatccagaattggtttgctcactcgcatcagtgggcaggtggtgcggactcacccagttcacccagagcttgtgagcggaacttttctgtgcttggactgtcagacagtgatcagggatgtagaacagcagttcaaatacacacagccaaacatctgccgaaatccagtttgtgccaacaggaggagattcttactggatacaaataaatcaagatttgttgattttcaaaaggttcgtattcaagagacccaagctgagcttcctcgagggagtatcccccgcagtttagaagtaattttaagggctgaagctgtggaatcagctcaagctggtgacaagtgtgactttacagggacactgattgttgtgcctgacgtctccaagcttagcacaccaggagcacgtgcagaaactaattcccgtgtcagtggtgttgatggatatgagacagaaggcattcgaggactccgggcccttggtgttagggacctttcttataggctggtctttcttgcctgctgtgttgcgccaaccaacccaaggtttggggggaaagagctcagagatgaggaacagacagctgagagcattaagaaccaaatgactgtgaaagaatgggagaaagtgtttgagatgagtcaagataaaaatctataccacaatctttgtaccagcctgttccctactatacatggcaatgatgaagtaaaacggggtgtcctgctgatgctctttggtggcgttccaaagacaacaggagaagggacctctcttcgaggggacataaatgtttgcattgttggtgacccaagtacagctaagagccaatttctcaagcacgtggaggagttcagccccagagctgtctacaccagtggtaaagcgtccagtgctgctggcttaacagcagctgttgtgagagatgaagaatctcatgagtttgtcattgaggctggagctttgatgttggctgataatggtgtgtgttgtattgatgaatttgataagatggacgtgcgggatcaagttgctattcatgaagctatggaacagcagaccatatccatcactaaagcaggagtgaaggctactctgaacgcccggacgtccattttggcagcagcaaacccaatcagtggacactatgacagatcaaaatcattgaaacagaatataaatttgtcagctcccatcatgtcccgattcgatctcttctttatccttgtggatgaatgtaatgaggttacagattatgccattgccaggcgcatagtagatttgcattcaagaattgaggaatcaattgatcgtgtctattccctcgatgatatcagaagatatcttctctttgcaagacagtttaaacccaagatttccaaagagtcagaggacttcattgtggagcaatataaacatctccgccagagagatggttctggagtgaccaagtcttcatggaggattacagtgcgacagcttgagagcatgattcgtctctctgaagctatggctcggatgcactgctgtgatgaggtccaacctaaacatgtgaaggaagctttccggttactgaataaatcaatcatccgtgtggaaacacctgatgtcaatctagatcaagaggaagagatccagatggaggtagatgagggtgccggtggcatcaatggtcatgctgacagccctgctcctgtgaacgggatcaatggctacaatgaagacataaatcaagagtctgctcccaaagcctccttaaggctgggcttctctgagtactgccgaatctctaaccttattgtgcttcacctcagaaaggtggaagaagaagaggacgagtcagcattaaagaggagcgagcttgttaactggtacttgaaggaaatcgaatcagagatagactctgaagaagaacttataaataaaaaaagaatcatagagaaagttattcatcgactcacacactatgatcatgttctaattgagctcacccaggctggattgaaaggctccacagagggaagtgagagctatgaagaagatccctacttggtagttaaccctaactacttgctcgaagattgagatagtgaaagtaactgaccagagctgaggaactgtggcacagcacctcgtggcctggagcctggctggagctctgctagggacagaagtgtttctggaagtgatgcttccaggatttgttttcagaaacaagaattgagttgatggtcctatgtgtcacattcatcacaggtttcataccaacacaggcttcagcacttcctttggtgtgtttcctgtcccagtgaagttggaaccaaataatgtgtagtctctataaccaatacctttgttttcatgtgtaagaaaaggcccattacttttaaggtatgtgctgtcctattgagcaaataactttttttcaattgccagctactgcttttattcatcaaaataaaataacttgttctgaagttgtctattggatttctttctactgtaccctgattattacttccatctacttctgaatgtgagactttccctttttgcttaacctggagtgaagaggtagaactgtggtattatggatgaggtttctatgagaaggagtcattagagaactcatatgaaagctagaggccttagagatgactttccaaggttaattccagttttttttttttttaagtttataaaagtttattatacttttttaaaattactctttagtaatttattttacttctgtgtcctaagggtaatttctcaggattgttttcaaattgcttttttaggggaaataggtcatttgctatattacaagcaatccccaaattttatggtcttccaggaaaagttattaccgtttatgatactaacagttcctgagacttagctatgatcagtatgttcatgaggtggagcagttcctgtgttgcagcttttaacaacagatggcattcattaaatcacaaagtatgttaaaggtcacaaaagcaaaataactgtctgaggctaaggcccacgtgggacagtctaatacccatgagtactcaacttgccttgatgtctgagctttccagtgcaatgtgaatttgagcagccagaaatctattagtagaaagcaagacagattaatataggttaaaacaatgatttaaatatgtttctcccaataattatctctttccctggaatcaacttgtatgaaaccttgtcaaaatgtactccacaagtatgtacaattaagtattttaaaaataaatggcaaacattaaaaaaaaaaaaaaaaaaaaaaaaaa

SEQ ID NO: 31 - DNA, 3321 bp, Homo sapiens
ttgtgagtctataactcggagccgttgggtcggttcctgctattccggcgcctccactccgtcccccgcgggtctgctctgtgtgccatggacggcattgtcccagatatagccgttggtacaaagcggggatctgacgagcttttctctacttgtgtcactaacggaccgtttatcatgagcagcaactcggcttctgcagcaaacggaaatgacagcaagaagttcaaaggtgacagccgaagtgcaggcgtcccctctagagtgatccacatccggaagctccccatcgacgtcacggagggggaagtcatctccctggggctgccctttgggaaggtcaccaacctcctgatgctgaaggggaaaaaccaggccttcatcgagatgaacacggaggaggctgccaacaccatggtgaactactacacctcggtgacccctgtgctgcgcggccagcccatctacatccagttctccaaccacaaggagctgaagaccgacagctctcccaaccaggcgcgggcccaggcggccctgcaggcggtgaactcggtccagtcggggaacctggccttggctgcctcggcggcggccgtggacgcagggatggcgatggccgggcagagccccgtgctcaggatcatcgtggagaacctcttctaccctgtgaccctggatgtgctgcaccagattttctccaagttcggcacagtgttgaagatcatcaccttcaccaagaacaaccagttccaggccctgctgcagtatgcggaccccgtgagcgcccagcacgccaagctgtcgctggacgggcagaacatctacaacgcctgctgcacgctgcgcatcgacttttccaagctcaccagcctcaacgtcaagtacaacaatgacaagagccgtgactacacacgcccagacctgccttccggggacagccagccctcgctggaccagaccatggccgcggccttcggtgcacctggtataatctcagcctctccgtatgcaggagctggtttccctcccacctttgccattcctcaagctgcaggcctttccgttccgaacgtccacggcgccctggcccccctggccatcccctcggcggcggcggcagctgcggcggcaggtcggatcgccatcccgggcctggcgggggcaggaaattctgtattgctggtcagcaacctcaacccagagagagtcacaccccaaagcctctttattcttttcggcgtctacggtgacgtgcagcgcgtgaagatcctgttcaataagaaggagaacgccctagtgcagatggcggacggcaaccaggcccagctggccatgagccacctgaacgggcacaagctgcacgggaagcccatccgcatcacgctctcgaagcaccagaacgtgcagctgccccgcgagggccaggaggaccagggcctgaccaaggactacggcaactcacccctgcaccgcttcaagaagccgggctccaagaacttccagaacatattcccgccctcggccacgctgcacctctccaacatcccgccctcagtctccgaggaggatctcaaggtcctgttttccagcaatgggggcgtcgtcaaaggattcaagttcttccagaaggaccgcaagatggcactgatccagatgggctccgtggaggaggcggtccaggccctcattgacctgcacaaccacgacctcggggagaaccaccacctgcgggtctccttctccaagtccaccatctaggggcacaggcccccacggccgggccccctggcgacaacttccatcattccagagaaaagccactttaaaaacagctgaagtgaccttagcagaccagagattttatttttttaaagagaaatcagtttacctgtttttaaaaaaattaaatctagttcaccttgctcaccctgcggtgacagggacagctcaggctcttggtgactgtggcagcgggagttcccggccctccacacccggggccagaccctcggggccatgccttggtggggcctgtgtcgggcgtggggcctgcaggtgggcgccccgaccacgacttggcttccttgtgccttaaaaaacctgccttcctgcagccacacacccacccggggtgtcctggggacccaaggggtgggggggtcacaccagagagaggcagggggcctggccggctcctgcaggatcatgcagctggggcgcggcggccgcggctgcgacaccccaaccccagccctctaatcaagtcacgtgattctcccttcaccccgcccccagggccttcccttctgcccccaggcgggctccccgctgctccagctgcggagctggtcgacataatctctgtattatatactttgcagttgcagacgtctgtgcctagcaatatttccagttgaccaaatattctaatcttttttcatttatatgcaaaagaaatagttttaagtaactttttatagcaagatgatacaatggtatgagtgtaatctaaacttccttgtggtattaccttgtatgctgttacttttattttattccttgtaattaagtcacaggcaggacccagtttccagagagcaggcggggccgcccagtgggtcaggcacagggagccccggtcctatcttagagcccctgagcttcagggaaggggcgggcgtgtcgccgcctctggcatcgcctccggttgccttacaccacgccttcacctgcagtcgcctagaaaacttgctctcaaacttcagggttttttcttccttcaaattttggaccaaagtctcatttctgtgttttgcctgcctctgatgctgggacccggaaggcgggcgctcctcctgtcttctctgtgctctttctaccgcccccgcgtcctgtcccgggggctctcctaggatcccctttccgtaaaagcgtgtaacaagggtgtaaatatttataattttttatacctgttgtgagacccgaggggcggcggcgcggttttttatggtgacacaaatgtatattttgctaacagcaattccaggctcagtattgtgaccgcggagccacaggggaccccacgcacattccgttgccttacccgatggcttgtgacgcggagagaaccgattaaaaccgtttgagaaactcctcccttgtctagccctgtgttcgctgtggacgctgtagaggcaggttggccagtctgtacctggacttcgaataaatcttctgtatcctcgctccgttccgccttaaaaaaaaaaaaaaaaaaaaaaaaaaaaaaaaaaaaaaaaaaaaaaaaaaaaaaaaaaa

SEQ ID NO: 32 - DNA, 1209 bp, Homo sapiens
gaattcctgacttccttttcggaggaagatccttgagcagccgacgttgggacaaaggatttggagaaacccagggctaaagtcacgtttttcctcctttaagacttacctcaacacttcactccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaaggcaccttcgtggagcgggaccgcaagcgggagaagaggaagcccaagagccaggagaccccggccaccaagaaggctgtgcaaggcgggggagccacccccgtggtgggggctgtccaggggcctgtcccgggcatgccgccgatgactcaggcgccccgcattatgcaccacatgccgggccagccgccctacatgccgccccctggtatgatccccccgccaggccttgcacctggccagatcccaccaggggccatgcccccgcagcagcttatgccaggacagatgccccctgcccagcctctttctgagaatccaccgaatcacatcttgttcctcaccaacctgccagaggagaccaacgagctcatgctgtccatgcttttcaatcagttccctggcttcaaggaggtccgtctggtacccgggcggcatgacatcgccttcgtggagtttgacaatgaggtacaggcaggggcagctcgcgatgccctgcagggctttaagatcacgcagaacaacgccatgaagatctcctttgccaagaagtagcaccttttccccccatgcctgccccttcccctgttctggggccacccctttcccccttggctcagccccctgaaggtaagtccccccttgggggccttcttggagccgtgtgtgagtgagtggtcgccacacagcattgtacccagagtctgtccccagacattgcacctggcgctgttaggccggaattaaagtggctttttgaggtttggtttttcacaaaaaaaaggaattc

SEQ ID NO: 33 - DNA, 1432 bp, Homo sapiens
gctgttcggcctgcgtcgctccgggagctgccgacggacggagcgcccccgcccccgcccggccgcccgcccgccgccgccatgcccttctccaacagccacaacgcactgaagctgcgcttcccggccgaggacgagttccccgacctgagcgcccacaacaaccacatggccaaggtgctgacccccgagctgtacgcggagctgcgcgccaagagcacgccgagcggcttcacgctggacgacgtcatccagacaggcgtggacaacccgggccacccgtacatcatgaccgtgggctgcgtggcgggcgacgaggagtcctacgaagtgttcaaggatctcttcgaccccatcatcgaggaccggcacggcggctacaagcccagcgatgagcacaagaccgacctcaaccccgacaacctgcagggcggcgacgacctggaccccaactacgtgctgagctcgcgggtgcgcacgggccgcagcatccgtggcttctgcctccccccgcactgcagccgcggggagcgccgcgccatcgagaagctcgcggtggaagccctgtccagcctggacggcgacctggcgggccgatactacgcgctcaagagcatgacggaggcggagcagcagcagctcatcgacgaccacttcctcttcgacaagcccgtgtcgcccctgctgctggcctcgggcatggcccgcgactggcccgacgcccgcggtatctggcacaatgacaataagaccttcctggtgtgggtcaacgaggaggaccacctgcgggtcatctccatgcagaaggggggcaacatgaaggaggtgttcacccgcttctgcaccggcctcacccagattgaaactctcttcaagtctaaggactatgagttcatgtggaaccctcacctgggctacatcctcacctgcccatccaacctgggcaccgggctgcgggcaggtgtgcatatcaagctgcccaacctgggcaagcatgagaagttctcggaggtgcttaagcggctgcgacttcagaagcgaggcacaggcggtgtggacacggctgcggtgggcggggtcttcgacgtctccaacgctgaccgcctgggcttctcagaggtggagctggtgcagatggtggtggacggagtgaagctgctcatcgagatggagcagcggctggagcagggccaggccatcgacgacctcatgcctgcccagaaatgaagcccggcccacacccgacaccagccctgctgcttcctaacttattgcctgggcagtgcccaccatgcacccctgatgttcgccgtctggcgagcccttagccttgctgtagagacttccgtcacccttggtagagtttatttttttgatggctaagatactgctgatgctgaaataaactagggttttggcctgcctgcgtctg

SEQ ID NO: 34 - DNA, 3309 bp, Homo sapiens
gcggcgcgcccgagcctagtccccacgccgcggcgcgcccgggctccctgctgatcccagaacaatcaaccatgacgaccgaatctggatcagactcggaatccaagccggaccaggaggccgagccccaggaggcggcgggggcgcaggggcggcgggggccgtgccggagccgcccaaggaggagcagcagcaggccctggagcagttcgccgccgctgcagcgcacagcaccccggtgcgagggaggtcactgacaaggaacaggagtttgctgccagggctgcaaaacagctcgaatatcagcaattagaagacgataaactttctcagaaatcatctagcagtaaactctctcggtctccattaaagattgtcaaaaagcctaaaagcatgcagtgcaaagtgatacttctcgatggatcagaatatacctgtgatgtagagaaacgctccagaggacaagtgctgtttgataaagtgtgtgaacacttgaacttgctagagaaagactactttgggcttacgtatcgagatgctgaaaaccagaagaattggttggaccctgctaaggaaataaaaaaacaggttcgaagtggtgcttggcacttttcatttaatgtgaaattttatccaccagaccctgcccaactatctgaagatatcaccaggtactacctctgcttgcagttgcgagatgacatcgtgtccggaaggctgccctgctcctttgttaccctggccttgctgggctcctacactgtccagtcagagctcggagactatgacccagatgaatgtgggagcgattacattagtgagttccgctttgcaccaaaccacactaaagaactggaagacaaagtgatcgagctgcacaagagccacagaggaatgacgccagcagaagcagagatgcatttcttggaaaatgccaaaaaattatcaatgtatggggtagatttacatcatgctaaggactcagaaggggtagaaattatgttaggagtttgtgcaagtggtctgttgatatatcgcgaccggctgcgaataaacagatttgcctggcccaaggttctaaagatttcatacaaacggaacaacttttacattaagatccggccgggagagtttgaacaatttgaaagcaccattgggtttaagctgccaaaccatcgagctgccaagcgtttatggaaagtatgtgttgagcatcatacatttttcagactactgttaccagaagcacctcccaagaaattcctaaccttgggttccaagtttcgttatagtggcaggacacaagcgcaaacgagaagagccagtgcgttgatagatcgcccagcaccttactttgaacgctcatccagcaaacgttataccatgtctcgcagcttggatggagcatcagtgaatgaaaaccatgaaatatacatgaaggattctatgtctgctgcagaggttggtactggccagtacgccacaacaaaaggcatctctcagaccaacttgatcaccactgtgactccggagaagaaggctgaggaggagcgggacgaggaagaggacaaacggaggaagggggaagaagtcacgcccatctcggccatccagcacgagggaaagactgacagtgagcgcacggacaccgcagccgacggggagaccactgccactgaggagctagaaaaaactcaagatgacctgatgaaacatcaaaccaacattagcgagctgaaaagaaccttcttagaaacctcaacagacactgccgtaacgaatgaatgggagaagaggctttccacctcccccgtgcgactggccgccaggcaggaggatgcccccatgatcgaaccacttgtccctgaagagaaaatggaaaccaagacggagtccagtggatagagacggaacccaccgtgcaccacctgccgcttagcactgagaaggtggtgcaggagaccgtgttggtggaggagcggcgtgtggtgcacgcgagtggggatgcttcttactcggcgggagacagcggggatgctgcagcacagcccgcattcacaggcattaaagggaaagagggctctgcttgacggagggggctaaagaggaaggaggggaggaggtcgctaaagctgtcctggaacaggaagagacagccgctgcttcccgtgagcgacaagaggagcagagtgcagccatccacatttcagaaactttggaacaaaaacctcattttgagtcctcaacggtgaagacggaaaccatcagttttggcagtgtttcaccgggaggagtaaagctagaaatttccacaagaagtgccagtagttcacaccgaaaccaaaaccatcacatatgaatcatcacaggtcgatccaggcacagatctggagccaggcgtgctgatgagtgcacagacgatcacatctgaaaccaccagtaccaccaccactacccacatcaccaaaactgtgaaagggggcatttcagagacaagaattgagaagcgaatagtcatcacgggggatgcagacattgaccatgaccaggcgctggctcaggcaattaaagaggccaaagagcagcaccctgacatgtcagtgaccaaagtagtggtccataaagagacagagatcacaccagaagatggagaggattgaccagaggaataacttagcttgcacatgaatgcagtcatgcaaaccgttaggaaaaccagagcctatatggagttccctcttctaacccaactgtacttgtatctgtccgtggaaaatttcagtccagaagaattgaccttgaccattaataaagacactggcagagagatcttcccataataaagcaatctgattcagcatcactaaaccgataatgcatgaagcaacgataaaattacaaaagagcagcatttttaattttcacaaaatgtctcagttttcagctatacctgctcgttcataaccaacaatataaaccgtggtctcatgtaacacataaacaattcatgcctttcatagtttattattattaaagtctaaacaaaattgcaatttcttaggtaaccttatatttacaataaatgaagattaccctcaaatgctagaagctgtctaggtccgtccggtgtgtcagatttcctcagattagatgtgccaataaccaagtttattcagtaaacaacttgtacttgtttcatctggtttattactctcacccataaacagtaatgactctctgaccctctggaaatatgtaatgcttccaatcttgctttgtgtatctcatttaatttgttccggttaagga

SEQ ID NO: 35 - DNA, 1195 bp, Homo sapiens
ggcacgaggcgccagtcccctaaccctgaggctgccgcgcggcggtcactgcgccggggtagtgggccccagtgttgcgctctctggccgttccttacactttgcttcaggctccagtgcaggggcgtagtgggatatggccaactcgggctgcaaggacgtcacgggtccagatgaggagagttttctgtactttgcctacggcagcaacctgctgacagagaggatccacctccgaaacccctcggcggcgttcttctgtgtggcccgcctgcaggattttaagcttgactttggcaattcccaaggcaaaacaagtcaaacttggcatggagggatagccaccatttttcagagtcctggcgatgaagtgtggggagtagtatggaaaatgaacaaaagcaatttaaattctctggatgagcaagaaggggttaaaagtggaatgtatgttgtaatagaagttaaagttgcaactcaagaaggaaaagaaataacctgtcgaagttatctgatgacaaattacgaaagtgctcccccatccccacagtataaaaagattatttgcatgggtgcaaaagaaaatggtttgccgctggagtatcaagagaagttaaaagcaatagaaccaaatgactatacaggaaaggtctcagaagaaattgaagacatcatcaaaaagggggaaacacaaactctttagaacataacagaatatatctaagggtattctatgtgctaatataaaatatttttaacacttgagaacagggatctgggggatctccacgtttgatccgttttcagcagtgctctgaaggagtatcttacttgggtgattccttgtttttagactataaaaagaaactgggataggagttagacaatttaaaaggggtgtatgagggcctgaaatatgtgacaaatgaatgtgagtaccccttctgtgaacactgaaagctattctcttgaattgatcttaagtgtctccttgctctggtaaaagatagatttgtagctcacttgatgatggtgctggtgaattgctctgctctgtctgagatttttaaaaatcagcttaatgagagtaatctgcagacaattgataataacattttgaaaattggaaagatggtatactgtttttagaggaataaacgtatttgtggtttaaaaaaaaaaaaaaaaaaaaaaa

SEQ ID NO: 36 - DNA, 2035 bp, Homo sapiens
gaattccgggctccggggatgaggtcgcggccggcgggtcccgcgctgttgctgctgctgctcttcctcggagcggccgagtcggtgcgtcgggcccagcctccgcgccgctacaccccagactggccgagcctggattctcggccgctgccggcctggttcgacgaagccaagttcggggtgttcatccactggggcgtgttctcggtgcccgcctggggcagcgagtggttctggtggcactggcagggcgaggggcggccgcagtaccagcgcttcatgcgcgacaactacccgcccggcttcagctacgccgacttcggaccgcagttcactgcgcgcttcttccacccggaggagtgggccgacctcttccaggccgcgggcgccaagtatgtagttttgacgacaaagcatcacgaaggcttcacaaactggccgagtcctgtgtcttggaactggaactccaaagacgtggggcctcatcgggatttggttggtgaattgggaacagctctccggaagaggaacatccgctatggactataccactcactcttagagtggttccatccactctatctacttgataagaaaaatggcttcaaaacacagcattttgtcagtgcaaaaacaatgccagagctgtacgaccttgttaacagctataaacctgatctgatctggtctgatggggagtgggaatgtcctgatacttactggaactccacaaattttctttcatggctctacaatgacagccctgtcaaggatgaggtggtagtaaatgaccgatggggtcagaactcttcctgtcaccatggaggatactataactgtgaagataaattcaagccacagagcttgccagatcacaagtgggagatgtgcaccagcattgacaagttttcctggggctatcgtcgtgacatggcattgtctgatgttacagaagaatctgaaatcatttcggaactggttcagacagtaagtttgggaggcaactatcttctgaacattggaccaactaaagatggactgattgttcccatcttccaagaaaggcttcttgctgttgggaaatggctgagcatcaatggggaggctatctatgcctccaaaccatggcgggtgcaatgggaaaagaacacaacatctgtatggtatacctcaaagggatcggctgtttatgccatttttctgcactggccagaaaatggagtcttaaaccttgaatcccccataactacctcaactacaaagataacaatgctgggaattcaaggagatctgaagtggtccacagatccagataaaggtctcttcatctctctaccccagttgccaccctctgctgtccccgcagagtttgcttggactataaagctgacaggagtgaagtaatcatttgagtgcaagaagaaagaggcgctgctcactgttttcctgcttcagtttttctcttatagtaccatcactataatcaacgaacttctcttctccacccagagatggcttttccaacacattttaattaaaggaactgagtacattaccctgatgtctaaatggaccaaagatctgagatccattgtgattatatctgtatcaggtcagcagaagaaggaactgagcagttgaactctgagttcatcaattctaatatttggaaattatctacaatggaatcttccctctgttctctgataacctacttgcttactcaatgcctttaagccaagtcaccctgttgcctatgggaggaggtggaaggatttggcaagctcaaccacatgctatttagttagcatcagttgtcaccaacagtctttctgcaaagggcaggagagctttgggggaaaggaaaaggcttaccaggctgctatggtcaactcttcagaaattttcagagcaatctaaaagcgccaaaattcgctatgtttacagtgatactattaagaaaatgaatgtgattctgctctgtctttttaagtatgatcaaataaaaaatttgtacatcacaatcatttctacc

SEQ ID NO: 37 - DNA, 2133 bp, Homo sapiens
cgggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaatccaccaaccagaacgtggtttgcctgaggctgtaactgagagaaagattctggggctgtcttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctcccctttgttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatctttcaaaataaaaagtaatgacttagaaactgaaaaaaaaaaaaaaaaaaaaaaaaaaaaaa

SEQ ID NO: 38 - DNA, 20 bp, Homo sapiens
agggaggaagggaaaacaga

SEQ ID NO: 39 - DNA, 20 bp, Homo sapiens
ttaaggctcaacacgaggct

SEQ ID NO: 40 - DNA, 20 bp, Homo sapiens
cttgagctgtgaggtcatcg

SEQ ID NO: 41 - DNA, 20 bp, Homo sapiens
tatagctcggcaccttcacc

SEQ ID NO: 42 - DNA, 21 bp, Homo sapiens
ctgcctgccactgagggttcc

SEQ ID NO: 43 - DNA, 24 bp, Homo sapiens
tccaggcagaacaacaaaccatcc

SEQ ID NO: 44 - DNA, 20 bp, Homo sapiens
accaccaccactacccacat

SEQ ID NO: 45 - DNA, 20 bp, Homo sapiens
tggttttcctaacggtttgc

SEQ ID NO: 46 - DNA, 21 bp, Homo sapiens
tgttggcgtacaggtctttgc

SEQ ID NO: 47 - DNA, 19 bp, Homo sapiens
gctacgagctgcctgacgg

SEQ ID NO: 48 - DNA, 24 bp, Homo sapiens
cacattaggctgttggttcaaact

SEQ ID NO: 49 - DNA, 19 bp, homo sapiens
caggatgcgctgaccactt

SEQ ID NO: 50 - DNA, 19 bp, Homo sapiens
tcctcacgccctgctatca

SEQ ID NO: 51 - DNA, 22 bp, Homo sapiens
ttcaggatgtccaggcatatgt

SEQ ID NO: 52 - DNA, 20 bp, homo sapiens
tgtcctcatctggaacaagg

SEQ ID NO: 53 - DNA, 20 bp, homo sapiens
ggcaggagttctgtcctttg

SEQ ID NO: 54 - DNA, 20 bp, homo sapiens
tttacatccagaggcacgac

SEQ ID NO: 55 - DNA, 19 bp, homo sapiens
cacgatgtcagcaaacagg

SEQ ID NO: 56 - DNA, 20 bp, homo sapiens
caggaaggctatggctttgg

SEQ ID NO: 57 - DNA, 21 bp, homo sapiens
ccgtttcacacctgacacatg

SEQ ID NO: 58 - DNA, 22 bp, homo sapiens
gctggaccggaagtaggtttct

SEQ ID NO: 59 - DNA, 17 bp, homo sapiens
gccgctaccggaaatgc

SEQ ID NO: 60 - DNA, 20 bp, homo sapiens
gacccagtgcatccaacaga

SEQ ID NO: 61 - DNA, 20 bp, homo sapiens
gtgtgcgcgtaaagcttcac

SEQ ID NO: 62 - DNA, 22 bp, homo sapiens
cagtaacaaccaatgtgtgcag

SEQ ID NO: 63 - DNA, 20 bp, homo sapiens
tgccaaaacgacttttgaac

SEQ ID NO: 64 - DNA, 20 bp, homo sapiens
gttggaccctgctaaggaaa

SEQ ID NO: 65 - DNA, 20 bp, homo sapiens
cagatagttgggcagggtct

SEQ ID NO: 66 - DNA, 22 bp, homo sapiens
cactggcaaaacaatgcagact

SEQ ID NO: 67 - DNA, 22 bp, Homo sapiens
cgaccttgaccatctttggatt